SEQUENCE LISTING (8 sequences)

SEQ ID NO: 1 - DNA, 1662 bp, Artificial
  TPA-PfAMA1 (3D7) - gene with tpa leader
atgaagagagggctctgctgtgtgctgctgctgtgtggagcagtcttcgtttcgcccagccaggaaatccatgcccgattcagaagactcgaccagaactactgggagcacccttaccagaacagcgacgtgtatcggcccatcaacgagcacagagagcaccccaaagaatacgagtatcccctgcaccaggaacacacctaccagcaggaagatagcggcgaggacgagaacaccctgcagcacgcctaccccatcgaccacgagggcgccgagcctgccccccaggaacagaacctgttcagcagcatcgagatcgtggagcggagcaactacatgggcaacccctggaccgagtatatggccaagtatgacatcgaggaagtgcacggcagcggcatccgggtggacctgggcgaggacgccgaggtggccggcacccagtatcggctgcccagcggcaagtgccccgtgttcggcaagggcatcatcatcgagaacagcaagaccaccttcctgacccccgtggccaccggcaatcagtatctgaaggacggcggcttcgccttcccccccaccgagcccctgatgagccccatgaccctggacgagatgcggcacttctacaaggacaacaagtatgtgaagaacctggacgagctgaccctgtgcagccggcacgccggcaacatgatccccgacaacgacaagaacagcaactacaagtatcccgccgtgtatgacgacaaggataagaagtgccacatcctgtatatcgccgcccaggaaaacaacggccccagatactgcaacaaggacgagagcaagcggaacagcatgttctgcttcagacccgccaaggacatcagcttccagaacctagtctacctgagcaagaacgtggtggacaactgggagaaagtgtgcccccggaagaacctgcagaacgccaagttcggcctgtgggtggacggcaactgcgaggacatcccccacgtgaacgagttccccgccatcgacctgttcgagtgcaacaagctggtgttcgagctgtccgccagcgaccagcccaagcagtatgagcagcacctgaccgactacgagaagatcaaagagggcttcaagaacaagaaccgcgagatgatcaagagcgccttcctgcccaccggcgccttcaaggccgacagatacaagagccacggcaagggctacaactggggcaactacaacaccgagacccagaagtgcgagatcttcaacgtgaagcccacctgcctgatcaatgacaagaactacatcgccaccaccgccctgagccaccccatcgaggtggagaacaacttcccctgcagcctgtataaggacgagatcatgaaagagatcgagcgggagagcaagaggatcaagctgaacgacaacgacgacgagggcaacaagaagatcatcgcccccaggatcttcatcagcgacgataaggacagcctgaagtgcccctgcgaccccgagatggtgtcccaaagtacatgccggttcttcgtgtgcaagtgcgtggagagaagggccgaggtgaccagcaacaacgaggtggtggtgaaagaggaatacaaggacgaatacgccgacatccccgagcacaagcccacctacgacaagatgaagtga

SEQ ID NO: 2 - DNA, 1224 bp, Artificial
  TPA-PyMSP142-PK - gene with tpa leader and PK tag
atggatgcaatgaagagagggctctgctgtgtgctgctgctgtgtggagcagtcttcgtttcgcccagccaggaaatccatgcccgattcagaagactcgactccgaagatgcaccagaaaaagatattctttccgaatttacaaatgaaagtttgtatgtatacacaaaaaggttgggtagtacatataaatcattaaagaaacacatgttaagagaattttcaacaattaaagaagacatgacaaatggattaaataataaatcacaaaaaagaaatgatttccttgaagtattaagccatgaattagatttattcaaagatttaagtaccaacaaatatgttattagaaatccatatcaattattagataatgataaaaaagacaaacaaatagtaaacttaaaatatgctactaaaggtataaatgaagatatagaaacaactactgacggaattaaattctttaacaaaatggttgaattatacaacactcaattagctgcagtaaaggaacaaattgctaccatagaagctgaaactaacgataccaataaagaagaaaaaaagaaatatattccaatccttgaagatcttaaaggattatatgaaaccgtaataggtcaagcagaagaatattcagaagaattacaaaatagacttgataattataaaaatgaaaaagctgaatttgaaatattaacaaaaaatttagaaaaatacatacaaattgacgaaaaacttgacgaatttgtagaacatgcagaaaataataaacacatagcctcaatagctttaaacaacttaaataaatctggtttagtaggagaaggtgaatcaaagaaaatattagcaaaaatgcttaacatggatggtatggatttattaggtgtagaccctaaacatgtatgtgttgatacaagagatattcctaaaaatgctggatgttttagagatgataatggtactgaagaatggagatgtttattaggttacaaaaaaggtgaaggtaatacatgtgtagaaaataataatcctacttgtgatatcaacaatggtggatgtgatccaactgctagttgtcaaaatgcggaaagtacggaaaattccaaaaaaattatatgtacatgtaaagaaccaacccctaatgcatattatgaaggtgtattctgtagttcttccagctttatgggaattcctaaccctttgctaggtctagactga

SEQ ID NO: 3 - DNA, 3120 bp, Artificial
  PfMSP1-15
atgaagatcatcttcttcctgtgctctttcctgttcttcatcatcaacacccagtgcgtgacccacgagagctaccaggagctggtgaagaagctggaggccctggaggacgccgtgctgaccggctacagcctgttccagaaagagaagatggtgctgaacgagctgttcgacctgaccaaccacatgctgaccctgtgcgacaacatccacggcttcaagtacctgatcgacggctacgaggagatcaacgagctgctgtacaagctgaacttctacttcgacctgctgcgcgccaagctgaacgacgtgtgcgccaacgactactgccagatccccttcaacctgaagatccgcgccaacgagctggacgtgctgaagaaactggtgttcggctaccggaagcccctggacaacatcaaggacaacgtgggcaagatggaggactacatcaagaagaacaagaccaccatcgccaacattaacgagctgatcgagggcagcaagaaaaccatcgaccagaacaagaacgccgacaacgaggagggcaagaagaagctgtaccaggcccagtacgacctgagcatctacaacaagcagctggaggaggcccacaacctgatcagcgtgctggagaagcggatcgacaccctgaagaagaacgagaacatcaagatcaaggagatcgccaagaccatcaagttcaacatcgactccctgttcaccgaccccctggagctggagtactacctgcgcgagaagaataagaagatgcagatcaagaagctgaccctgctgaaggagcagctggaaagcaagctgaacagcctgaacaacccccacaacgtgctgcagaacttcagcgtgttcttcaacaagaagaaggaggccgagatcgccgaaaccgagaacaccctggagaataccaagatcctgctgaagcactacaagggcctggtgaagtactacaacggcgagagcagccccctgaaaaccctgagcgaagtgagcatccagaccgaggacaactacgccaacctggagggccaagtggtcaccggcgaggccgtgaccacaagcgtgatcgacaatatcctgagcaagatcgagaacgagtacgaagtgctgtacctgaagcctctggccggcgtgtaccggagcctgaagaaacagctggagaacaacgtgatgaccttcaacgtgaacgtgaaggacatcctgaacagccggttcaacaagcgcgagaacttcaagaacgtgctggagtccgacctgatcccctacaaggacctgaccagcagcaactacgtggtgaaggacccctacaagttcctgaacaaggagaagcgcgacaagtttctgtccagctacaactacattaaggacagcatcgacaccgacatcaacttcgccaacgacgtgctgggctactacaagatcctgagcgagaagtacaagagcgacctggatagcatcaagaagtacatcaacgacaagcagggcgagaacgagaagtacctgcccttcctgaataacatcgagaccctgtacaagaccgtgaacgacaagatcgacctgttcgtgatccacctggaggccaaagtgctgaactacacctacgagaagagcaacgtggaagtgaagattaaggagctgaactacctgaaaaccatccaggacaagctggccgacttcaagaagaataacaacttcgtgggcatcgccgatctgagcaccgactacaaccacaacaacctgctgaccaagttcctgtccaccggcatggtgttcgagaacctgctgaagagcgtgctgagcaacctgctggactggaagctggcccgctacgtgaagcacttcaccacccccatgcggaaaaagaccatgatccagcagagcggagggggacccgggggaggggaccaagtcgtgaccggcgaagccatcagcgtgaccatggataacatcctgagcggcttcgaaaacgaatacgacgtgatctatctgaaacccctggccggcgtgtatcggtctctgaagaagcagatcgagaagaacatcttcaccttcaatctgaacctgaacgatatcctgaatagccgcctgaagaagcgcaagtacttcctggacgtgctggagagcgacctgatgcagttcaagcacatcagcagcaacgagtacatcatcgaggacagcttcaagctgctgaacagcgagcagaagaacacactgctgaagtcttacaagtatatcaaggagagcgtggagaacgatatcaagttcgcccaggagggcatcagctactacgagaaagtgctggccaagtacaaggacgatctggagtccatcaagaaagtgatcaaggaggagaaggagaagttccccagcagcccccccaccaccccccccagccccgccaagaccgacgagcagaagaaggagagcaagttcctgccttttctgaccaatatcgagacactgtataacaacctggtgaataagatcgacgactacctgatcaatctgaaggccaagatcaacgattgcaacgtggagaaggacgaggcccacgtgaagatcaccaagctgagcgatctgaaagccatcgacgataagatcgatctgttcaagaacccctacgacttcgaggccattaagaagctgatcaacgacgacaccaagaaggacatgctgggcaagctgctgtctaccggcctggtgcagaatttccccaacaccatcatcagcaagctgatcgaagggaagttccaggatatgctgaacatcgcccagcaccagtgcgtgaagaagcagatccccgagaacagcggctgcttccggcacctggacgagcgcgaggagtggaagtgcctgctgaattacaagcaggagggcgacaagtgcgtggagaatcccaaccccacctgcaacgagaacaacggcggctgcgacgccgacgccacctgcaccgaggaggacagcggcagcagccggaagaagatcacctgcgagtgcaccaagcccgacagctaccccctgttcgacggcatcttctgcagcagctccaacttaatattatacagcttcatcaagtacatccccatcctggaggacctgtga

SEQ ID NO: 4 - DNA, 720 bp, Artificial
  GFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 5 - DNA, 2373 bp, Artificial
  ME-TRAP
atgggtatgatcaacgcctacttggacaagttgatctccaagtacgaagacgaaatctcctacatcccatctgccgaaaagatcggatctaagccgaacgacaagtccttgtataaacctaaggacgaattggactacaagccaatcgttcaatacgacaacttcggatctgcctccaagaacaaggaaaaggctttgatcatcggtatcgctggtggtttggccttgttgatgaaccctaatgacccaaacagaaacgtcagatctcacttgggtaacgttaagtacttggttaagtctttgtacgatgaacacatcttattgatggactgttctggttctattggatctgacccaaacgctaacccaaacgttgacccaaacgccaacccaaacgtccaagttcacttccaaccattgcctccggccgttgtcaagttgcaattcatcaaggccaactctaagttcatcggtatcaccgaaggatcttacttgaacaaaattcaaaactctttgatggaaaagttgaaagaattggaaaaggctacttctgtcttggctggtttgggatctaacgctaatccaaacgcaaatccgaacgccaatcctaacgcgaatcccgacgaatggtctccatgttctgtcacttgtggtaagggtactcgctctagaaagagagaaggatccaaaataatgaatcatcttgggaatgttaaatatttagtcattgtgtttttgattttctttgatttgtttctagttaatggtagagatgtgcaaaacaatatagtggatgaaataaaatatagtgaagaagtatgtaatgatcaggtagatctttaccttctaatggattgttctggaagtatacgtcgtcataattgggtgaaccatgcagtacctctagctatgaaattgatacaacaattaaatcttaatgataatgcaattcacttatatgttaatgttttttcaaacaatgcaaaagaaattattagattacatagtgatgcatctaaaaacaaagagaaggctttaattattataaggtcactcttaagtacaaatcttccatatggtagaacaaacttaactgatgcactgttacaagtaagaaaacatttaaatgaccgaatcaatagagagaatgctaatcaattagttgttatattaacagatggaattccagatagtattcaagattcattaaaagaatcaagaaaattaagtgatcgtggtgttaaaatagctgtttttggtattggacaaggtattaatgtagctttcaacagatttcttgtaggttgtcatccatcagatggtaaatgtaacttgtatgctgattctgcatgggaaaatgtaaaaaatgttatcggaccctttatgaaggctgtttgtgttgaagtagaaaaaacagcaagttgtggtgtttgggacgaatggtctccatgtagtgtaacttgtggtaaaggtaccaggtcaagaaaaagagaaatcttacacgaaggatgtacaagtgaaatacaagaacaatgtgaagaagaaagatgtcctccaaaatgggaaccattagatgttccagatgaacccgaagatgatcaacctagaccaagaggagataattcttctgtccaaaaaccagaagaaaatataatagataataatccacaagaaccttcaccaaatccagaagaaggaaaggatgaaaatccaaacggatttgatttagatgaaaatccagaaaatccaccaaatccagatattcctgaacaaaaaccaaatatacctgaagattcagaaaaagaagtaccttctgatgttccaaaaaatccagaagacgatcgagaagaaaactttgatattccaaagaaacccgaaaataagcacgataatcaaaataatttaccaaatgataaaagtgatagaaatattccatattcaccattacctccaaaagttttggataatgaaaggaaacaaagtgacccccaaagtcaagataataatggaaataggcacgtacctaatagtgaagatagagaaacacgtccacatggtagaaataatgaaaatagatcatacaatagaaaatataacgatactccaaaacatcctgaaagggaagaacatgaaaagccagataataataaaaaaaaaggagaatcagataataaatataaaattgcaggtggaatagctggaggattagctttactcgcatgtgctggacttgcttataaattcgtagtaccaggagcagcaacaccctatgccggagaacctgcaccttttgatgaaacattaggtgaagaagataaagatttggacgaacctgaacaattcagattacctgaagaaaacgagtggaattaa

SEQ ID NO: 6 - DNA, 3333 bp, Artificial
  PfM128
atgaagatcatcttcttcctgtgctctttcctgttcttcatcatcaacacccagtgcgtgacccacgagagctaccaggagctggtgaagaagctggaggccctggaggacgccgtgctgaccggctacagcctgttccagaaagagaagatggtgctgaacgagctgttcgacctgaccaaccacatgctgaccctgtgcgacaacatccacggcttcaagtacctgatcgacggctacgaggagatcaacgagctgctgtacaagctgaacttctacttcgacctgctgcgcgccaagctgaacgacgtgtgcgccaacgactactgccagatccccttcaacctgaagatccgcgccaacgagctggacgtgctgaagaaactggtgttcggctaccggaagcccctggacaacatcaaggacaacgtgggcaagatggaggactacatcaagaagaacaagaccaccatcgccaacattaacgagctgatcgagggcagcaagaaaaccatcgaccagaacaagaacgccgacaacgaggagggcaagaagaagctgtaccaggcccagtacgacctgagcatctacaacaagcagctggaggaggcccacaacctgatcagcgtgctggagaagcggatcgacaccctgaagaagaacgagaacatcaagatcaaggagatcgccaagaccatcaagttcaacatcgactccctgttcaccgaccccctggagctggagtactacctgcgcgagaagaataagaagatgcagatcaagaagctgaccctgctgaaggagcagctggaaagcaagctgaacagcctgaacaacccccacaacgtgctgcagaacttcagcgtgttcttcaacaagaagaaggaggccgagatcgccgaaaccgagaacaccctggagaataccaagatcctgctgaagcactacaagggcctggtgaagtactacaacggcgagagcagccccctgaaaaccctgagcgaagtgagcatccagaccgaggacaactacgccaacctggagggccaagtggtcaccggcgaggccgtgacccccagcgtgatcgacaacatcctgagcaagatcgagaacgagtacgaggtgctgtacctgaagcccctggccggcgtgtacagaagcctgaagaagcagctggaaaacaacgtgatgaccttcaacgtgaacgtgaaggacatcctgaacagccggttcaacaagcgggagaacttcaagaacgtgctggaaagcgacctgatcccctacaaggacctgaccagcagcaactacgtggtgaaggacccctacaagttcctgaacaaagagaagcgggataagttcctgagcagctacaactacatcaaggacagcatcgacaccgacatcaacttcgccaacgacgtgctgggctactacaagatcctgagcgagaagtacaagagcgacctggacagcatcaagaagtacatcaacgacaagcagggcgagaacgagaagtacctgcccttcctgaataacatcgagaccctgtacaagaccgtgaacgacaagatcgacctgttcgtgatccacctggaagccaaggtgctgaactacacctacgagaagagcaacgtggaggtgaagatcaaagagctgaactacctgaaaaccatccaggacaagctggccgacttcaagaagaacaacaacttcgtcggcatcgccgacctgagcaccgactacaaccacaacaacctgctgaccaagttcctgtccaccggcatggtgttcgagaacctggccaagacagtgctgtccaacctgctggacggcaacctgcagggcatgctcaatatcgcacagcatcagtgtgtcaaaaaacagattcctcagaactccggctgctttagacacctggatgaacgggaagaatggaagtgtctgctcaactataaacaggaaggtgataagtgtgtcgagaaccctaaccctacctgtaatgagaataatgggggctgtgatgccgatgccaaatgtaccgaagaagattccggctccaatggcaagaaaatcacatgtgaatgtaccaaacccgactcctaccctctcttcgatgggatcttttgcagctccagtaatggcggcggacccgggggaggggaccaagtcgtgaccggcgaagccatcagcgtgaccatggataacatcctgagcggcttcgaaaacgaatacgacgtgatctatctgaaacccctggccggcgtgtatcggtctctgaagaagcagatcgagaagaacatcttcaccttcaatctgaacctgaacgatatcctgaatagccgcctgaagaagcgcaagtacttcctggacgtgctggagagcgacctgatgcagttcaagcacatcagcagcaacgagtacatcatcgaggacagcttcaagctgctgaacagcgagcagaagaacacactgctgaagtcttacaagtatatcaaggagagcgtggagaacgatatcaagttcgcccaggagggcatcagctactacgagaaagtgctggccaagtacaaggacgatctggagtccatcaagaaagtgatcaaggaggagaaggagaagttccccagcagcccccccaccaccccccccagccccgccaagaccgacgagcagaagaaggagagcaagttcctgccttttctgaccaatatcgagacactgtataacaacctggtgaataagatcgacgactacctgatcaatctgaaggccaagatcaacgattgcaacgtggagaaggacgaggcccacgtgaagatcaccaagctgagcgatctgaaagccatcgacgataagatcgatctgttcaagaacccctacgacttcgaggccattaagaagctgatcaacgacgacaccaagaaggacatgctgggcaagctgctgtctaccggcctggtgcagaatttccccaacaccatcatcagcaagctgatcgaagggaagttccaggatatgctgaacatcgcccagcaccagtgcgtgaagaagcagatccccgagaacagcggctgcttccggcacctggacgagcgcgaggagtggaagtgcctgctgaattacaagcaggagggcgacaagtgcgtggagaatcccaaccccacctgcaacgagaacaacggcggctgcgacgccgacgccacctgcaccgaggaggacagcggcagcagccggaagaagatcacctgcgagtgcaccaagcccgacagctaccccctgttcgacggcatcttctgcagcagctccaacttaatattatattcctttatctga

SEQ ID NO: 7 - DNA, 32790 bp, Human adenovirus type 5
taacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtcgaagcttggatccggtacctctagaattctcgagcggccgctagcgacatcgatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctccgttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatcgattcgacagatcactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcgaatcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaat

SEQ ID NO: 8 - DNA, 29416 bp, Simian adenovirus type 63
aaaccatcatcaataatatacctcaaacttttggtgcgcgttaatatgcaaatgagctgtttgaatttggggagggaggaaggtgattggctgcgggagcggcgaccgttaggggcggggcgggtgacgttttgatgacgtggctatgaggcggagccggtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtatttttcacctaaatttccgcgtacggtgtcaaagtccggtgtttttacgcgatcgctagcgacatcgatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctccgttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatcgattcgacagatcgcgatcgcgtgagtagtgtttggggctgggtgtgagcctgcatgaggggcagaatgactaaaatctgtggttttctgtgtgttgcagcagcatgagcggaagcgcctcctttgagggaggggtattcagcccttatctgacggggcgtctcccctcctgggcgggagtgtgtcagaatgttatgggatccacggtggacggccggcccgtgcagcccgcgaactcttcaaccctgacctacgcgaccctgagctcctcgtccgtggacgcagctgccgccgcagctgctgcttccgccgccagcgccgtgcgcggaatggccctgggcgccggctactacagctctctggtggccaactcgagttccaccaataatcccgccagcctgaacgaggagaagctgttgctgctgatggcccagctcgaggccctgacccagcgcctgggcgagctgacccagcaggtggctcagctgcaggcggagacgcgggccgcggttgccacggtgaaaaccaaataaaaaatgaatcaataaataaacggagacggttgttgattttaacacagagtcttgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcgtggtgctgcacgatgtctttgaggaggagactgatggccacgggcagccccttggtgtaggtgttgacgaacctattgagctgggagggatgcatgcggggggagatgagatgcatcttggcctggatcttgagattggcgatgttcccgcccagatcccgccgggggttcatgttgtgcaggaccaccagcacggtgtatccggtgcacttggggaatttgtcatgcaacttggaagggaaggcgtgaaagaatttggagacgcccttgtgaccgcccaggttttccatgcactcatccatgatgatggcgatgggcccgtgggcggcggcctgggcaaagacgtttcgggggtcggacacatcgtagttgtggtcctgggtgagctcgtcataggccattttaatgaatttggggcggagggtacccgactgggggacaaaggtgccctcgatcccgggggcgtagttcccctcgcagatctgcatctcccaggccttgagctcggagggggggatcatgtccacctgcggggcgatgaaaaaaacggtttccggggcgggggagatgagctgcgccgaaagcaggttccggagcagctgggacttgccgcagccggtggggccgtagatgaccccgatgaccggctgcaggtggtagttgagggagagacagctgccgtcctcgcggaggaggggggccacctcgttcatcatctcgcgcacatgcatgttctcgcgcacgagttccgccaggaggcgctcgccccccagcgagaggagctcttgcagcgaggcgaagtttttcagcggcttgagcccgtcggccatgggcattttggagagggtctgttgcaagagttccagacggtcccagagctcggtgatgtgctctagggcatctcgatccagcagacctcctcgtttcgcgggttggggcgactgcgggagtagggcaccaggcgatgggcgtccagcgaggccagggtccggtccttccagggtcgcagggtccgcgtcagcgtggtctccgtcacggtgaaggggtgcgcgccgggctgggcgcttgcgagggtgcgcttcaggctcatccggctggtcgagaaccgctcccggtcggcgccctgcgcgtcggccaggtagcaattgagcatgagttcgtagttgagcgcctcggccgcgtggcccttggcgcggagcttacctttggaagtgtgtccgcagacgggacagaggagggacttgagggcgtagagcttgggggcgaggaagacggactcgggggcgtaggcgtccgcgccgcagctggcgcagacggtctcgcactccacgagccaggtgaggtcggggcggtcggggtcaaaaacgaggtttcctccgtgctttttgatgcgtttcttacctctggtctccatgagctcgtgtccccgctgggtgacaaagaggctgtccgtgtccccgtagaccgactttatgggccggtcctcgagcggggtgccgcggtcctcgtcgtagaggaaccccgcccactccgagacgaaggcccgggtccaggccagcacgaaggaggccacgtgggaggggtagcggtcgttgtccaccagcgggtccaccttctccagggtatgcaagcacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccgttggagacgcctttcatgagcccctcgtccatctggtcagaaaagacgatctttttgttgtcgagcttggtggcgaaggagccgtagagggcgttggagagcagcttggcgatggagcgcatggtctggttcttttccttgtcggcgcgctccttggcggcgatgttgagctgcacgtactcgcgcgccacgcacttccattcggggaagacggtggtgagctcgtcgggcacgattctgacccgccagccgcggttgtgcagggtgatgaggtccacgctggtggccacctcgccgcgcaggggctcgttggtccagcagaggcgcccgcccttgcgcgagcagaaggggggcagcgggtccagcatgagctcgtcgggggggtcggcgtccacggtgaagatgccgggcaggagctcggggtcgaagtagctgatgcaggtgcccagatcgtccagacttgcttgccagtcgcgcacggccagcgcgcgctcgtaggggctgaggggcgtgccccagggcatggggtgcgtgagcgcggaggcgtacatgccgcagatgtcgtagacgtagaggggctcctggaggacgccgatgtaggtggggtagcagcgccccccgcggatgctggcgcgcacgtagtcgtacagctcgtgcgagggcgcgaggagccccgtgccgagattggagcgctgcggcttttcggcgcggtagacgatctggcggaagatggcgtgggagttggaggagatggtgggcctctggaagatgttgaagtgggcatggggcagtccgaccgagtccctgatgaagtgggcgtaggagtcctgcagcttggcgacgagctcggcggtgacgaggacgtccagggcgcagtagtcgagggtctcttggatgatgtcgtacttgagctggcccttctgcttccacagctcgcggttgagaaggaactcttcgcggtccttccagtactcttcgagggggaacccgtcctgatcggcacggtaagagcccaccatgtagaactggttgacggccttgtaggcgcagcagcccttctccacggggagggcgtaagcttgcgcggccttgcgcagggaggtgtgggtgagggcgaaggtgtcgcgcaccatgactttgaggaactggtgcttgaagtcgaggtcgtcgcagccgccctgctcccagagctggaagtccgtgcgcttcttgtaggcggggttgggcaaagcgaaagtaacatcgttgaagaggatcttgcccgcgcggggcatgaagttgcgagtgatgcggaaaggctggggcacctcggcccggttgttgatgacctgggcggcgaggacgatctcgtcgaagccgttgatgttgtgcccgacgatgtagagttccacgaatcgcgggcggcccttgacgtggggcagcttcttgagctcgtcgtaggtgagctcggcggggtcgctgagcccgtgctgctcgagggcccagtcggcgacgtgggggttggcgctgaggaaggaagtccagagatccacggccagggcggtctgcaagcggtcccggtactgacggaactgctggcccacggccattttttcgggggtgacgcagtagaaggtgcgggggtcgccgtgccagcggtcccacttgagctggagggcgaggtcgtgggcgagctcgacgagcggcgggtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccagttggaggaatggctgttgatgtgatggaagtagaaatgccgacggcgcgccgagcactcgtgcttgtgtttatacaagcgtccgcagtgctcgcaacgctgcacgggatgcacgtgctgcacgagctgtacctgggttcctttgacgaggaatttcagtgggcagtggagcgctggcggctgcatctggtgctgtactacgtcctggccatcggcgtggccatcgtctgcctcgatggtggtcatgctgacgagcccgcgcgggaggcaggtccagacctcggctcggacgggtcggagagcgaggacgagggcgcgcaggccggagctgtccagggtcctgagacgctgcggagtcaggtcagtgggcagcggcggcgcgcggttgacttgcaggagcttttccagggcgcgcgggaggtccagatggtacttgatctccacggcgccgttggtggcgacgtccacggcttgcagggtcccgtgcccctggggcgccaccaccgtgccccgtttcttcttgggcggcggcggctccatgcttagaagcggcggcgaggacgcgcgccgggcggcaggggcggctcggggcccggaggcaggggcggcaggggcacgtcggcgccgcgcgcgggcaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcggccggcgcgctcgacggtggccgcgaggtcgttggagatgcggcccatgagctgcgagaaggcgttcatgccggcctcgttccagacgcggctgtagaccacggctccgtcggggtcgcgcgcgcgcatgaccacctgggcgaggttgagctcgacgtggcgcgtgaagaccgcgtagttgcagaggcgctggtagaggtagttgagcgtggtggcgatgtgctcggtgacgaagaagtacatgatccagcggcggagcggcatctcgctgacgtcgcccagggcttccaagcgctccatggcctcgtagaagtccacggcgaagttgaaaaactgggagttgcgcgccgagacggtcaactcctcctccagaagacggatgagctcggcgatggtggcgcgcacctcgcgctcgaaggccccggggggctcctcttccatttcctcctcttcctcctccactaacatctcttctacttcctcctcaggaggcggcggcgggggaggggccctgcgtcgccggcggcgcacgggcagacggtcgatgaagcgctcgatggtctccccgcgccggcgacgcatggtctcggtgacggcgcgcccgtcctcgcggggccgcagcgtgaagacgccgccgcgcatctccaggtggccgccgggggggtctccgttgggcagggagagggcgctgacgatgcatcttatcaattgacccgtagggactccgcgcaaggacctgagcgtctcgagatccacgggatccgaaaaccgctgaacgaaggcttcgagccagtcgcagtcgcaaggtaggctgagcccggtttcttgttcttcgggtatttggtcgggaggcgggcgggcgatgctgctggtgatgaagttgaagtaggcggtcctgagacggcggatggtggcgaggagcaccaggtccttgggcccggcttgctggatgcgcagacggtcggccatgccccaggcgtggtcctgacacctggcgaggtccttgtagtagtcctgcatgagccgctccacgggcacctcctcctcgcccgcgcggccgtgcatgcgcgtgagcccgaacccgcgctgcggctggacgagcgccaggtcggcgacgacgcgctcggcgaggatggcctgctggatctgggtgagggtggtctggaagtcgtcgaagtcgacgaagcggtggtaggctccggtgttgatggtgtaggagcagttggccatgacggaccagttgacggtctggtggccggggcgcacgagctcgtggtacttgaggcgcgagtaggcgcgcgtgtcgaagatgtagtcgttgcaggtgcgcacgaggtactggtatccgacgaggaagtgcggcggcggctggcggtagagcggccatcgctcggtggcgggggcgccgggcgcgaggtcctcgagcatgaggcggtggtagccgtagatgtacctggacatccaggtgatgccggcggcggtggtggaggcgcgcgggaactcgcggacgcggttccagatgttgcgcagcggcaggaagtagttcatggtggccgcggtctggcccgtgaggcgcgcgcagtcgtggatgctctagacatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgctaacgaaacctccaggatacggaggcgggtcgttttttggccttggtcgctggtcatgaaaaactagtaagcgcggaaagcggccgcccgcgatggctcgctgccgtagtctggagaaagaatcgccagggttgcgttgcggtgtgccccggttcgagcctcagcgctcggcgccggccggattccgcggctaacgtgggcgtggctgccccgtcgtttccaagaccccttagccagccgacttctccagttacggagcgagcccctctttttttcttgtgtttttgccagatgcatcccgtactgcggcagatgcgcccccaccctccaccacaaccgcccctaccgcagcagcagcaacagccggcgcttctgcccccgccccagcagcagcagccagccactaccgcggcggccgccgtgagcggagccggcgttcagtatgacctggccttggaagagggcgaggggctggcgcggctgggggcgtcgtcgccggagcggcacccgcgcgtgcagatgaaaagggacgctcgcgaggcctacgtgcccaagcagaacctgttcagagacaggagcggcgaggagcccgaggagatgcgcgcctcccgcttccacgcggggcgggagctgcggcgcggcctggaccgaaagcgggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcacgctgatcgcgcgcgaggaggtgaccctgggcctgatgcacctgtgggacctgctggaggccatcgtgcagaaccccacgagcaagccgctgacggcgcagctgtttctggtggtgcagcacagtcgggacaacgagacgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagcctgggcaagtactacgctaggaagatctacaagaccccgtacgtgcccatagacaaggaggtgaagatcgatgggttttacatgcgcatgaccctgaaagtgctgaccctgagcgacgatctgggggtgtaccgcaacgacaggatgcaccgcgcggtgagcgccagccgccggcgcgagctgagcgaccaggagctgatgcacagcctgcagcgggccctgaccggggccgggaccgagggggagagctactttgacatgggcgcggacctgcgctggcagcccagccgccgggccttggaagctgccggcggcgtgccctacgtggaggaggtggacgatgaggaggaggagggcgagtacctggaagactgatggcgcgaccgtatttttgctagatgcagcaacagccaccgccgccgcctcctgatcccgcgatgcgggcggcgctgcagagccagccgtccggcattaactcctcggacgattggacccaggccatgcaacgcatcatggcgctgacgacccgcaatcccgaagcctttagacagcagcctcaggccaaccggctctcggccatcctggaggccgtggtgccctcgcgctcgaaccccacgcacgagaaggtgctggccatcgtgaacgcgctggtggagaacaaggccatccgcggcgacgaggccgggctggtgtacaacgcgctgctggagcgcgtggcccgctacaacagcaccaacgtgcagacgaacctggaccgcatggtgaccgacgtgcgcgaggcggtgtcgcagcgcgagcggttccaccgcgagtcgaacctgggctccatggtggcgctgaacgccttcctgagcacgcagcccgccaacgtgccccggggccaggaggactacaccaacttcatcagcgcgctgcggctgatggtggccgaggtgccccagagcgaggtgtaccagtcggggccggactacttcttccagaccagtcgccagggcttgcagaccgtgaacctgagccaggctttcaagaacttgcagggactgtggggcgtgcaggccccggtcggggaccgcgcgacggtgtcgagcctgctgacgccgaactcgcgcctgctgctgctgctggtggcgcccttcacggacagcggcagcgtgagccgcgactcgtacctgggctacctgcttaacctgtaccgcgaggccatcgggcaggcgcacgtggacgagcagacctaccaggagatcacccacgtgagccgcgcgctgggccaggaggacccgggcaacctggaggccaccctgaacttcctgctgaccaaccggtcgcagaagatcccgccccagtacgcgctgagcaccgaggaggagcgcatcctgcgctacgtgcagcagagcgtggggctgttcttgatgcaggagggggccacgcccagcgccgcgctcgacatgaccgcgcgcaacatggagcccagcatgtacgcccgcaaccgcccgttcatcaataagctgatggactacttgcatcgggcggccgccatgaactcggactactttaccaacgccatcttgaacccgcactggctcccgccgcccgggttctacacgggcgagtacgacatgcccgaccccaacgacgggttcctgtgggacgacgtggacagcagcgtgttctcgccgcggcccaccaccaccaccgtgtggaagaaagagggcggggaccggcggccgtcctcggcgctgtccggtcgcgcgggtgctgccgcggcggtgcccgaggctgccagccccttcccgagcctgcccttttcgctgaacagcgtgcgcagcagcgagctgggtcggctgacgcggccgcgcctgctgggcgaggaggagtacctgaacgactccttgttgaagcccgagcgcgagaagaacttccccaataacgggatagagagcctggtggacaagatgagccgctggaagacgtacgcgcacgagcacagggacgagccccgagctagcagcgcaggcacccgtagacgccagcggcacgacaggcagcggggactggtgtgggacgatgaggattccgccgacgacagcagcgtgttggacttgggtgggagtggtggtggtaacccgttcgctcacctgcgcccccgtatcgggcgcctgatgtaagaatctgaaaaaataaaagacggtactcaccaaggccatggcgaccagcgtgcgttcttctctgttgtttgtagtagtatgatgaggcgcgtgtacccggagggtcctcctccctcgtacgagagcgtgatgcagcaggcggtggcggcggcgatgcagcccccgctggaggcgccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaacgatttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcgcgcaagacccccaacggggtcacggtaggggatgattatgatggtagtcaggacgagctgacctacgagtgggtggagtttgagctgcccgagggcaacttctcggtgaccatgaccatcgatctgatgaacaacgccatcatcgacaactacttggcggtggggcggcagaacggggtgctggagagcgacatcggcgtgaagttcgacacgcgcaacttccggctgggctgggaccccgtgaccgagctggtgatgccgggcgtgtacaccaacgaggccttccaccccgacatcgtcctgctgcccggctgcggcgtggacttcaccgagagccgcctcagcaacctgctgggcatccgcaagcggcagcccttccaggagggcttccagatcctgtacgaggacctggaggggggcaacatccccgcgctcttggatgtcgaagcctatgaagaaagtaaggaaaaagcagaggctgaggcaactacagccgtggctaccgccgcgactgtggcagatgccactgtcaccaggggcgatacattcgccacccaggcggaggaagcagccgccctagcggcgaccgatgatagtgaaagtaagatagtcatcaagccggtggagaaggacagcaagaacaggagctacaacgttctaccggatggaaagaacaccgcctaccgcagctggtacctggcctacaactacggcgaccccgagaagggcgtgcgctcctggacgctgctcaccacctcggacgtcacctgcggcgtggagcaagtctactggtcgctgcccgacatgatgcaagacccggtcaccttccgctccacgcgacaagttagcaactacccggtggtgggcgccgagctcctgcccgtctactccaagagcttcttcaacgagcaggccgtctactcgcagcagctgcgtgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgtcactgacgccagacgccgcacctgcccctacgtctacaaggccctgggcgtagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcgctcgcgcaccaccgtcgacgacgtgatcgaccaggtggtggccgacgcgcgcaactacacgcccgccgccgcgcccgcctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcctccggcagcagcagcgccggcaggacccgcagacgcgcggccacggcggcggcggcggccatcgccagcatgtcccgcccgcggcgcggcaacgtgtactgggtgcgcgacgccgccaccggtgtgcgcgtgcccgtgcgcacccgcccccctcgcacttgaagatgctgacttcgcgatgttgatgtgtcccagcggcgaggaggatgtccaagcgcaaatacaaggaagagatgctccaggtcatcgcgcctgagatctacggccccgcggcggcggtgaaggaggaaagaaagccccgcaaactgaagcgggtcaaaaaggacaaaaaggaggaggaagatgacggactggtggagtttgtgcgcgagttcgccccccggcggcgcgtgcagtggcgcgggcggaaagtgaaaccggtgctgcggcccggcaccacggtggtcttcacgcccggcgagcgttccggctccgcctccaagcgctcctacgacgaggtgtacggggacgaggacatcctcgagcaggcggtcgagcgtctgggcgagtttgcttacggcaagcgcagccgccccgcgcccttgaaagaggaggcggtgtccatcccgctggaccacggcaaccccacgccgagcctgaagccggtgaccctgcagcaggtgctgccgagcgcggcgccgcgccggggcttcaagcgcgagggcggcgaggatctgtacccgaccatgcagctgatggtgcccaagcgccagaagctggaggacgtgctggagcacatgaaggtggaccccgaggtgcagcccgaggtcaaggtgcggcccatcaagcaggtggccccgggcctgggcgtgcagaccgtggacatcaagatccccacggagcccatggaaacgcagaccgagcccgtgaagcccagcaccagcaccatggaggtgcagacggatccctggatgccagcggcttccaccaccaccactcgccgaagacgcaagtacggcgcggccagcctgctgatgcccaactacgcgctgcatccttccatcatccccacgccgggctaccgcggcacgcgcttctaccgcggctacaccagcagccgccgccgcaagaccaccacccgccgccgtcgtcgcagccgccgcagcagcaccgcgacttccgccttggtgcggagagtgtatcgcagcgggcgcgagcctctgaccctgccgcgcgcgcgctaccacccgagcatcgccatttaactaccgcctcctacttgcagatatggccctcacatgccgcctccgcgtccccattacgggctaccgaggaagaaagccgcgccgtagaaggctgacggggaacgggctgcgtcgccatcaccaccggcggcggcgcgccatcagcaagcggttggggggaggcttcctgcccgcgctgatccccatcatcgccgcggcgatcggggcgatccccggcatagcttccgtggcggtgcaggcctctcagcgccactgagacacaaaaaagcatggatttgtaataaaaaaatggactgacgctcctggtcctgtgatgtgtgtttttagatggaagacatcaatttttcgtccctggcaccgcgacacggcacgcggccgtttatgggcacctggagcgacatcggcaacagccaactgaacgggggcgccttcaattggagcagtctctggagcgggcttaagaatttcgggtccacgctcaaaacctatggcaacaaggcgtggaacagcagcacagggcaggcgctgagggaaaagctgaaagagcagaacttccagcagaaggtggtcgatggcctggcctcgggcatcaacggggtggtggacctggccaaccaggccgtgcagaaacagatcaacagccgcctggacgcggtcccgcccgcggggtccgtggagatgccccaggtggaggaggagctgcctcccctggacaagcgcggcgacaagcgaccgcgtcccgacgcggaggagacgctgctgacgcacacggacgagccgcccccgtacgaggaggcggtgaaactgggtctgcccaccacgcggcccgtggcgcctctggccaccggggtgctgaaacccagcagcagcagccagcccgcgaccctggacttgcctccgcctgcttcccgcccctccacagtggctaagcccctgccgccggtggccgtcgcgtcgcgcgccccccgaggccgcccccaggcgaactggcagagcactctgaacagcatcgtgggtctgggagtgcagagtgtgaagcgccgccgctgctattaaaagacactgtagcgcttaacttgcttgtctgtgtgtgtatatgtatgtccgccgaccagaaggaggaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccccagcttcaaaccctactccggcaccgcctacaacagcctagctcccaagggagcgcccaacacctcacagtggaaggattccgacagcaaaatgcatacttttggagttgctgccatgcccggtgttgttggtaaaaaaatagaagccgatggtctgcctattggaatagattcatcctctggaactgacaccataatttatgctgataaaactttccaaccagagccacaggttggaagtgacagttgggtcgacaccaatggtgcagaggaaaaatatggaggtagagctcttaaggacactacaaacatgaagccctgctacggttcttttgccaggcctaccaacaaagaaggtggacaggctaacataaaagattctgaaactgccagcactactcctaactatgatatagatttggcattctttgacagcaaaaatattgcagctaactacgatccagatattgtaatgtacacagaaaatgttgagttgcaaactccagatactcatattgtgtttaagccaggaacttcagatgaaagttcagaagccaatttgggccagcaggccatgcccaacagacccaactacatcgggttcagagacaactttatcgggctcatgtactacaacagcactggcaatatgggtgtactggctggtcaggcctcccagctaaatgctgtggtggacttgcaggacagaaacaccgaactgtcctaccagctcttgcttgactctctgggtgacagaaccaggtatttcagtatgtggaatcaggcggtggacagctatgaccccgatgtgcgcattattgaaaatcacggtgtggaggatgaactccccaattattgcttccctttgaatggtgtaggctttacagatacttaccagggtgttaaagttaagacagatacagccgctactggtaccaatggaacgcagtgggacaaagatgataccacagtcagcactgccaatgagatccactcaggcaatcctttcgccatggagatcaacatccaggccaacctgtggcggaacttcctctacgcgaacgtggcgctgtacctgcccgactcctacaagtacacgccggccaacatcacgctgccgaccaacaccaacacctacgattacatgaacggccgcgtggtggcgccctcgctggtggacgcctacatcaacatcggggcgcgctggtcgctggaccccatggacaacgtcaaccccttcaaccaccaccgcaacgcgggcctgcgctaccgctccatgctcctgggcaacgggcgctacgtgcccttccacatccaggtgccccaaaagtttttcgccatcaagagcctcctgctcctgcccgggtcctacacctacgagtggaacttccgcaaggacgtcaacatgatcctgcagagctccctcggcaacgacctgcgcacggacggggcctccatcgccttcaccagcatcaacctctacgccaccttcttccccatggcgcacaacaccgcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggatggtccttcacgcgcctcaagacccgcgagacgccctcgctcggctccgggttcgacccctacttcgtctactcgggctccatcccctacctcgacggcaccttctacctcaaccacaccttcaagaaggtctccatcaccttcgactcctccgtcagctggcccggcaacgaccgcctcctgacgcccaacgagttcgaaatcaagcgcaccgtcgacggagagggatacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtcgtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcgccagcgtcacccagaaaaagttcctctgcgaccgggtcatgtggcgcatccccttctccagcaacttcatgtccatgggcgcgctcaccgacctcggccagaacatgctctacgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacgcccttctcggccggcaacgccaccacctaaagccccgctcttgcttcttgcaagatgacggcctgtggctccggcgagcaggagctcagggccatcctccgcgacctgggctgcgggccctgcttcctgggcaccttcgacaagcgcttcccgggattcatggccccgcacaagctggcctgcgccatcgtcaacacggccggccgcgagaccgggggcgagcactggctggccttcgcctggaacccgcgctcccacacctgctacctcttcgaccccttcgggttctcggacgagcgcctcaagcagatctaccagttcgagtacgagggcctgctgcgccgcagcgccctggccaccgaggaccgctgcatcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaacgcccactccgcctactttcgctcccaccgcgcgcgcatcgagaaggccaccgccttcgaccgcatgaatcaagacatgtaaactgtgtgtatgtgaatgctttattcatcataataaacagcacatgtttatgccaccttctctgaggctctgactttatttagaaatcgaaggggttctgccggctctcggcgtgccccgcgggcagggatacgttgcggaactggtacttgggcagccacttgaactcggggatcagcagcttcggcacggggaggtcggggaacgagtcgctccacagcttgcgcgtgagttgcagggcgcccagcaggtcgggcgcggagatcttgaaatcgcagttgggacccgcgttctgcgcgcgagagttgcggtacacggggttgcagcactggaacaccatcagggccgggtgcttcacgctcgccagcaccgtcgcgtcggtgatgccctccacgtccagatcctcggcgttggccatcccgaagggggtcatcttgcaggtctgccgccccatgctgggcacgcagccgggcttgtggttgcaatcgcagtgcagggggatcagcatcatctgagcctgctcggagctcatgcccgggtacatggccttcatgaaagcctccagctggcggaaggcctgctgcgccttgccgccctcggtgaagaagaccccacaggacttgctagagaactggttggtggcgcagcccgcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcgtgtgctccttctggatcatcacggtcccgtgcaggcaccgcagcttgccctcggcctcggtgcacccgtgcagccacagcgcgcagccggtgcactcccagttcttgtgggcgatctgggagtgcgagtgcacgaagccctgcaggaagcggcccatcatcgtggtcagggtcttgttgctggtgaaggtcagcgggatgccgcggtgctcctcgttcacatacaggtggcagatgcggcggtacacctcgccctgctcgggcatcagctggaaggcggacttcaggtcgctctccacgcggtaccgctccatcagcagcgtcatcacttccatgcccttctcccaggccgaaacgatcggcaggctcagggggttcttcaccgtcatcttagtcgccgccgccgaagtcagggggtcgttctcgtccagggtctcaaacactcgcttgccgtccttctcggtgatgcgcacggggggaaagctgaagcccacggccgccagctcctcctcggcctgcctttcgtcctcgctgtcctggctgatgtcttgcaaaggcacatgcttggtcttgcggggtttctttttgggcggcagaggcggcggcggagacgtgctgggcgagcgcgagttctcgctcaccacgactatttcttcttcttggccgtcgtccgagaccacgcggcggtaggcatgcctcttctggggcagaggcggaggcgacgggctctcgcggttcggcgggcggctggcagagccccttccgcgttcgggggtgcgctcctggcggcgctgctctgactgacttcctccgcggccggccattgtgttctcctagggagcaacaagcatggagactcagccatcgtcgccaacatcgccatctgcccccgccgccgacgagaaccagcagcagcagaatgaaagcttaaccgccccgccgcccagccccacctccgacgccgccgcggccccagacatgcaagagatggaggaatccatcgagattgacctgggctacgtgacgcccgcggagcacgaggaggagctggcagcgcgcttttcagccccggaagagaaccaccaagagcagccagagcaggaagcagagagcgagcagcagcaggctgggctcgagcatggcgactacctgagcggggcagaggacgtgctcatcaagcatctggcccgccaaagcatcatcgtcaaggacgcgctgctcgaccgcgccgaggtgcccctcagcgtggcggagctcagccgcgcctacgagcgcaacctcttctcgccgcgcgtgccccccaagcgccagcccaacggcacctgcgagcccaacccgcgcctcaacttctacccggtcttcgcggtgcccgaggccctggccacctaccacctctttttcaagaaccaaaggatccccgtctcctgccgcgccaaccgcacccgcgccgacgccctgctcaacctgggtcccggcgcccgcctacctgatatcacctccttggaagaggttcccaagatcttcgagggtctgggcagcgacgagactcgggccgcgaacgctctgcaaggaagcggagaggagcatgagcaccacagcgccctggtggagttggaaggcgacaacgcgcgcctggcggtgctcaagcgcacggtcgagctgacccacttcgcctacccggcgctcaacctgccccccaaggtcatgagcgccgtcatggaccaggtgctcatcaagcgcgcctcgcccctctcagaggaggagatgcaggaccccgagagctcggacgagggcaagcccgtggtcagcgacgagcagctggcgcgctggctgggagcgagcagcaccccccagagcctggaagagcggcgcaagctcatgatggccgtggtcctggtgaccgtggagctggagtgtctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcctgcacgagaaccgcctggggcagaacgtgctgcacaccaccctgcgcggggaggcccgccgcgactacatccgcgactgcgtctacctgtacctctgccacacctggcagacgggcatgggcgtgtggcagcagtgcctggaggagcagaacctgaaagagctctgcaagctcctgcagaagaacctcaaggccctgtggaccgggttcgacgagcgcaccaccgcctcggacctggccgacctcatcttccccgagcgcctgcggctgacgctgcgcaacgggctgcccgactttatgagccaaagcatgttgcaaaactttcgctctttcatcctcgaacgctccgggatcctgcccgccacctgctccgcactgccctcggacttcgtgccgctgaccttccgcgagtgccccccgccgctctggagccactgctacttgctgcgcctggccaactacctggcctaccactcggacgtgatcgaggacgtcagcagcgagggtctgctcgagtgccactgccgctgcaacctctgcacgccgcaccgctccttggcctgcaacccccagctgctgagcgagacccagatcatcggcaccttcgagttgcaaggccccggcgagggcaaggggggtctcaaactcaccccggggctgtggacctcggcctacttgcgcaagttcgtgcccgaggactaccatcccttcgagatcaggttctacgaggaccaatcccagccgcccaaggccgagctgtcggcctgcgtcatcacccagggggccatcctggcccaattgcaagccatccagaaatcccgccaagaatttctgctgaaaaagggccacggggtctacttggacccccagaccggagaggagctcaaccccagcttcccccaggatgccccgaggaagcagcaagaagctgaaagtggagctgccgctgccgccggaggatttggaggaagactgggagagcagtcaggcagaggagatggaagactgggacagcactcaggcagaggaggacagcctgcaagacagtctggaggaggaagacgaggtggaggaggaggcagaggaagaagcagccgccgccagaccgtcgtcctcggcggaggagaaagcaagcagcacggataccatctccgctccgggtcggggtcgcggcggccgggcccacagtagatgggacgagaccgggcgcttcccgaaccccaccacccagaccggtaagaaggagcggcagggatacaagtcctggcgggggcacaaaaacgccatcgtctcctgcttgcaagcctgcgggggcaacatctccttcacccggcgctacctgctcttccaccgcggggtgaacttcccccgcaacatcttgcattactaccgtcacctccacagcccctactactgtttccaagaagaggcagaaacccagcagcagcagcagaaaaccagcggcagcagcagcagctagaaaatccacagcggcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccacacacggaaaaaggcgggaattacgtcaccacctgcgcccttcgcccgaccatcatcatgagcaaagagattcccacgccttacatgtggagctaccagccccagatgggcctggccgccggcgccgcccaggactactccacccgcatgaactggctcagtgccgggcccgcgatgatctcacgggtgaatgacatccgcgcccaccgaaaccagatactcctagaacagtcagcgatcaccgccacgccccgccatcaccttaatccgcgtaattggcccgccgccctggtgtaccaggaaattccccagcccacgaccgtactacttccgcgagacgcccaggccgaagtccagctgactaactcaggtgtccagctggccggcggcgccgccctgtgtcgtcaccgccccgctcagggtataaagcggctggtgatccgaggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggcggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatggtggcgcggctgacctagctcggcttcgacacctggaccactgttaattaatcgcctctcctacgagctcctgcagcagcgccagaagttcacctgcctggtcggagtcaaccccatcgtcatcacccagcagtcgggcgataccaaggggtgcatccactgctcctgcgactcccccgactgcgtccacactctgatcaagaccctctgcggcctccgcgacctcctccccatgaactaatcacccccttatccagtgaaataaagatcatattgatgatgattttacagaaataaagatacaatcatattgatgatttgagtttaataaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgctgtccctgcgactggccgaccccgtcaccaccaagaacggggaaatcaccctcaagctgggagagggggtggacctcgactcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcgccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaaaaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcacaattgggagaagtactcgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagtcttggcgcgccaaagtctagaagcggtccatagcttaccgagcggcagcagcagcggcacacaacaggcgcaagagtcagagaaaagactgagctctaacctgtccgcccgctctctgctcaatatatagccccagatctacactgacgtaaaggccaaagtctaaaaatacccgccaaataatcacacacgcccagcacacgcccagaaaccggtgacacactcagaaaaatacgcgcacttcctcaaacggccaaactgccgtcatttccgggttcccacgctacgtcatcaaaacacgactttcaaattccgtcgaccgttaaaaacatcacccgccccgcccctaacggtcgccgctcccgcagccaatcaccttcctccctccccaaattcaaacagctcatttgcatattaacgcgcaccaaaagtttgaggtatattattgatgatgggttt

(19) United States
(12) Patent Application Publication
(10) Pub. No.: US 2014/0127258 A1
(43) Pub. Date: May 8, 2014

(54) Viral Vector Immunogenic Compositions

(71) Applicants: Simone de Cassan, Oxford (GB); Alexander Douglas, Oxford (GB); Simon Draper, Oxford (GB); Emily Forbes, Oxford (GB); Anna Goodman, Oxford (GB); Adrian Hill, Oxford (GB); Antia Millicic, Oxford (GB); Arturo Reyes-Sandoval, Oxford (GB)

(72) Inventors: Simone de Cassan, Oxford (GB); Alexander Douglas, Oxford (GB); Simon Draper, Oxford (GB); Emily Forbes, Oxford (GB); Anna Goodman, Oxford (GB); Adrian Hill, Oxford (GB); Antia Millicic, Oxford (GB); Arturo Reyes-Sandoval, Oxford (GB)

(73) Assignee: Isis Innovation Limited, Oxford (GB)

(21) Appl. No.: 13/877,002

(22) PCT Filed: Sep. 30, 2011
(86) PCT No.: PCT/GB11/51865

§ 371 Date: Jun. 19, 2013

(30) Foreign Application Priority Data
Sep. 30, 2010 (GB) 1016471.3

(51) Int. Cl. C12N 15/86; A61K 39/015

(52) U.S. Cl. CPC C12N 15/86; A61K 39/015
USPC 424/199.1

(57) ABSTRACT

There is provided a composition comprising: (a) a modified vaccinia virus ankara (MVA) vector, wherein said MVA vector comprises a nucleic acid sequence encoding an antigen; and (b) an adjuvant comprising a saponin, or an emulsion. There is also provided a composition comprising: (a) an adenovirus vector, wherein said adenovirus vector comprises a nucleic acid sequence encoding an antigen, and wherein the adenovirus is selected from: a group B adenovirus, a group C adenovirus, and a group E adenovirus; and (b) an adjuvant comprising a saponin, or an emulsion; wherein the group B adenovirus is not an adenovirus 35, the group C adenovirus is not Ad5 having an intact E3 gene region, and the group E adenovirus is not an adenovirus C7. Also provided are corresponding uses of the compositions in medicine.

This patent application claims priority to GB 1016471.3 filed on 30 Sep. 2011, which is hereby incorporated by reference in its entirety.

The present invention relates to compositions comprising an adenovirus vector and/or an MVA vector together with an adjuvant and their use as immunogenic compositions.

Recombinant viral vectors encoding antigens from infectious pathogens are being studied for use in vaccines. Viral vectored vaccines serve as antigen delivery vehicles and also have the power to activate the innate immune system through binding cell surface molecules that recognise viral elements.

As a consequence of their intrinsic immunostimulatory properties, but also because it has been very difficult to identify any adjuvant that can enhance their immunogenicity safely and reliably, viral vectors have generally been used without an adjuvant.

Historically, adjuvants were developed in order to improve the immunogenicity and efficacy of protein vaccines, in many cases aiming to mimic the effect of the viral activation of the immune system.

Previous work supports the notion that some adjuvants can diminish the efficacy of viral vectored vaccines in animal models, due to the mutual interference of their effects on the immune system, involving inhibitory cytokine interactions between interferons and IL-1β (Masters, S. L. et al., EMBO, Rep 11, 640-646). Furthermore, interferons are known for their general anti-viral activity and adjuvants that induce IFN production could be expected to inhibit the immunogenicity of viral vectored vaccines through the anti-viral effects of type I interferons.

There is a need for immunogenic compositions that demonstrate improved immunogenicity when used in the prevention or treatment of infectious diseases such as malaria, HIV/AIDS and tuberculosis without increasing the risk of reactogenicity.

There is therefore a need for improved viral vector compositions that can be used in immunogenic compositions. In particular, there is a need for improved viral vectored immunogenic compositions that can be used to produce an improved antigen specific T cell response, and additionally an improved antibody response.

The present invention addresses the above need by providing compositions and uses of such compositions in medicine, including in the prevention and treatment of at least one infectious disease.

The compositions of the present invention provide increased immunogenicity and efficacy when used to stimulate an immune response in a subject, allowing for the use of reduced doses. Such increased immunogenicity and efficacy is achieved through the combination of specific types of viral vector and specific types of adjuvants, optionally further combined with a polypeptide antigen.

In one aspect, the invention provides a composition comprising (a) a modified vaccinia virus ankara (MVA) vector, wherein said MVA vector comprises a nucleic acid sequence encoding an antigen; and (b) an adjuvant comprising a saponin, or an emulsion.

The genomic sequence of modified vaccinia virus ankara is detailed in Antoine et al. (Virology. 1998 May 10; 244(2):365-96; this publication is hereby incorporated by reference in its entirety).

The present inventors have found that combining certain specific adjuvants with an MVA vector as described above produces a composition that surprisingly can elicit an increased immunological response when administered to a subject.

The compositions of the present invention are particularly suited for use in medicine and in stimulating or inducing an immunological response in a subject. A composition of the present invention may be employed to stimulate or induce an immune response in a subject, either alone or in combination with another composition of the invention. The compositions of the present invention may be employed in a variety of immunisation protocols, as detailed below

The viral vectors employed in the present invention may be non-replicating. As used herein, a non-replicating viral vector is a viral vector which lacks the ability to replicate following infection of a target cell. Thus, the viral vector used in the invention cannot produce additional copies of itself.

MVA has been found not to replicate in almost all mammalian cell lines and does not productively replicate when used to immunise mammals. It is thus regarded as a non-replicating viral vector. Other examples of non-replicating poxyiral vectors include NYVAC, and avipox vectors such as ALVAC vectors.

As detailed below, adenovirus vectors may also be employed in the present invention. Adenoviruses can be rendered non-replicating by deletion of the E1 or both the E1 and E3 gene regions. Alternatively, an adenovirus may be rendered non-replicating by alteration of the E1 or of the E1 and E3 gene regions such that said gene regions are rendered non-functional. For example, a non-replicating adenovirus may lack a functional E1 region or may lack functional E1 and E3 gene regions. In this way the adenoviruses are rendered replication incompetent in most mammalian cell lines and do not replicate in immunised mammals. Most preferably, both E1 and E3 gene region deletions are present in the adenovirus, thus allowing a greater size of transgene to be inserted. This is particularly important to allow larger antigens to be expressed, or when multiple antigens are to be expressed in a single vector, or when a large promoter sequence, such as the CMV promoter, is used. Deletion of the E3 as well as the E1 region is particularly favoured for recombinant Ad5 vectors. Optionally, the E4 region can also be engineered.

In one embodiment, the composition comprises a non-replicating MVA vector.

In one embodiment, the MVA vector of the invention is intact—i.e. it does not comprise any gene deletions as compared with standard MVA.

In one embodiment, the MVA vector of the invention has an intact A26L gene.

The MVA vector comprises a nucleic acid sequence encoding an antigen. Subject to the size constraints imposed by the MVA vector, the antigen encoded may be any antigen.

In one embodiment, the antigen encoded by the nucleic acid sequence is a polypeptide.

In one embodiment, the antigen encoded by the nucleic acid sequence is an antigen from a pathogenic organism. Examples of suitable antigens include, but are not limited to, a malaria antigen, a tuberculosis antigen, an influenza antigen or an HIV antigen.

In one embodiment, the antigen encoded by the nucleic acid sequence is a malaria antigen, for example an antigen based on merozoite surface protein 1 (MSP1). In one embodiment, the antigen encoded by the viral vector is based on MSP1 from Plasmodium falciparum, for example PfM115 [described by Goodman A L, Epp C, Moss D, et al. Infect Immun. 2010 Aug. 16.], PfMSP1₁₅, PfMSP1₁₉, PfMSP1₃₃, and PfMSP1₄₂. In one embodiment, the antigen is Plasmodium yoelii MSP1. Further, non-limiting, examples of suitable malaria antigens include apical membrane antigen-1 (AMA1); ME.TRAP (the TRAP sequence of P. falciparum, attached to a multi-epitope (ME) string that expresses Pb9, an H2K(d)-restricted epitope (SYIPSAEKI) that is immunodominant in BALB/c mice); and PfM128. Other examples of suitable antigens include antigens derived from P. falciparum and/or P. vivax, for example wherein the antigen is selected from DBP, PvTRAP, PvMSP2, PvMSP4, PvMSP5, PvMSP6, PvMSP7, PvMSP8, PvMSP9, PvAMAI and RBP or fragment thereof. Other example antigens derived from P. falciparum include, PfEMP-I, Pfs 16 antigen, MSP-I, MSP-3, LSA-I, LSA-3, AMA-I and TRAP. Other Plasmodium antigens include P. falciparum EBA, GLURP, RAPI, RAP2, Sequestrin, PO32, STARP, SALSA, PfEXPI, Pfs25, PfHAP2, Pfs28, PFS27/25, Pfs48/45, Pfs230 and their analogues in other Plasmodium spp. Antigens from the mosquito vector of malaria may also be used where it may be desirable to block transmission of malaria, e.g the APN1 antigen.

In one embodiment, the antigen encoded by the nucleic acid sequence is the antigen encoded by the nucleic acid sequence of any one of SEQ ID NOs: 1-6.

In one embodiment, the antigen encoded by the nucleic acid is an antigen selected from the group consisting of: a Plasmodia antigen, an influenza virus antigen, a Mycobacterium tuberculosis antigen, a Mycobacterium bovis antigen, a Mycobacteria antigen, a hepatitis C virus antigen, a flavivirus antigen, a hepatitis B virus antigen, a human immunodeficiency virus antigen, a retrovirus antigen, a Staphylococcus aureus antigen, a Staphylococci antigen, a Streptococcus pneumoniae antigen, a Streptococcus pyogenes antigen, a Streptococci antigen, a Haemophilus influenzae antigen, and a Neisseria meningitides antigen.

In one embodiment, the antigen encoded by the nucleic acid sequence is not a Chlamydia sp. (e.g. C. trachomatis or C. pneumoniae) antigen.

The compositions of the present invention (as described above) comprise an adjuvant comprising a saponin, or an emulsion.

In one embodiment, the adjuvant is a saponin.

In one embodiment, the saponin is a Quill A fraction, for example QS21.

In one embodiment, the adjuvant comprising a saponin is ISCOM Matrix.

ISCOMs are “immune stimulating complexes”. ISCOM Matrix adjuvant comprises a mixture of saponins and other organic compounds such as phospholipids and cholesterol that form cage-like particles. In more detail, ISCOM Matrix comprises purified saponins obtained from a crude extract of the plant Quillaja saponaria Molina, cholesterol from Lanolin and phosphatidyl choline. This adjuvant is a suspension of nano-sized (40 nm) cage-like particles consisting of the above ingredients in PBS.

Examples of ISCOM Matrix adjuvants are ISCOM Matrix-M and Abisco-100 (Isconova, Sweden).

An emulsion may be an oil-in-water, water-in-oil, or water-in-oil-in-water emulsion. The emulsion may comprise a mineral oil and/or a non-mineral oil.

In one embodiment, the emulsion is selected from Montanide ISA720, Montanide ISA206, Emulsigen, and Titermax.

In one embodiment, the emulsion is selected from Montanide ISA720, Montanide ISA206, Emulsigen, Titermax, and MF59.

Montanide ISA720 (Seppic, France) is a squalene-based water-in-oil emulsion. In more detail, Montanide ISA720 comprises squalene (non-mineral metabolisable oil) and refined emulsifier/surfactant based on mannide oleate. Montanide ISA720 is designed to be used as a water-in-oil (W/0) emulsion when combined with antigen.

Montanide ISA206 (Seppic, France) is an emulsion comprising mannide oleate and mineral oil. In more detail, Montanide ISA206 comprises mineral oil (non-metabolisable) and is designed to be used as water-in-oil-in-water (W/O/W) emulsion with antigen.

Emulsigen (MVP Technologies) is an oil-in-water emulsion. In more detail, Emulsigen comprises a mineral oil-in-water (0/W) stable emulsion of particle size 1-2 microns.

Titermax (TiterMax, CytRx Corporation) is a water-in-oil emulsion comprising a block copolymer CRL-8941, squalene, a metabolisable oil, and a microparticulate stabilizer. TiterMax may alternatively contain a block copolymer CRL-8300, squalene (non-mineral metabolisable oil) and a microparticulate stabiliser.

MF59 (Novartis) is a squalene oil-in-water emulsion.

In one embodiment, the composition does not comprise a TLR (Toll-Like Receptor) ligand. TLRs are form a class of receptors that play an important role in the innate immune system The present inventors have found that, in certain circumstances, the absence of a TLR ligand from a composition of the present invention surprisingly leads to an improvement in the immune response elicited when the composition is administered to a subject.

Thus, in one embodiment, the composition is formulated as described above but lacks the presence of any additional component able to bind to and stimulate a TLR receptor.

In one embodiment, the composition when administered to a subject does not stimulate a TLR-mediated response.

In one embodiment, the composition further comprises a polypeptide antigen. In one embodiment, the presence of a polypeptide antigen means that, following administration of the composition to a subject, a simultaneous T cell and antibody response may be achieved. In one embodiment, the T cell and antibody response achieved surpasses that achieved when either a viral vector or polypeptide antigen are used alone.

In one embodiment, the polypeptide antigen is a polypeptide antigen from a pathogenic organism. Examples of suitable antigens include, but are not limited to, a malaria polypeptide antigen, a tuberculosis polypeptide antigen, an influenza polypeptide antigen, or an HIV polypeptide antigen.

In one embodiment, the polypeptide antigen is a malaria antigen. Examples of suitable malaria antigens include, but are not limited to, an antigen based on merozoite surface protein 1 (MSP1), such as an antigen based on MSP1 from Plasmodium falciparum, for example PfM115, PfMSP1₁₅, PfMSP1₁₉, PfMSP1₃₃, and PfMSP1₄₂; Plasmodium yoelii MSP1; apical membrane antigen-1 (AMA1); ME.TRAP (the TRAP sequence of P. falciparum, attached to a multi-epitope (ME) string that expresses Pb9, an H2K(d)-restricted epitope (SYIPSAEKI) that is immunodominant in BALB/c mice); PfM128. Other examples of suitable antigens include antigens derived from derived from P. falciparum and/or P. vivax, for example wherein the antigen is selected from DBP, PvTRAP, PvMSP2, PvMSP4, PvMSP5, PvMSP6, PvMSP7, PvMSP8, PvMSP9, PvAMAI and RBP or fragment thereof. Other example antigens derived from P. falciparum include, PfEMP-I, Pfs 16 antigen, MSP-I, MSP-3, LSA-I, LSA-3, AMA-I and TRAP. Other Plasmodium antigens include P. falciparum EBA, GLURP, RAPI, RAP2, Sequestrin, PO32, STARP, SALSA, PfEXPI, Pfs25, Pfs28, PFS27/25, Pfs48/45, Pfs230 and their analogues in other Plasmodium spp. Antigens from the mosquito vector of malaria may also be used where it may be desirable to block transmission of malaria, e.g the APN1 antigen.

In one embodiment, the polypeptide antigen is the antigen encoded by the nucleic acid sequence of any one of SEQ ID NOs: 1-6.

In one embodiment, the composition further comprises a polypeptide antigen wherein the polypeptide antigen is different from the antigen encoded by the nucleic acid sequence. Thus, in one embodiment, administration of the composition to a subject can elicit a simultaneous immune response against different antigens, for example a T cell response against the antigen encoded by the nucleic acid sequence of the viral vector and an antibody response against the polypeptide antigen.

In one embodiment, the polypeptide antigen is an antigen from a pathogenic organism.

In one embodiment, the polypeptide antigen is not covalently bonded to the MVA vector. In one embodiment, the polypeptide antigen is a separate component to the MVA vector.

In one embodiment, the antigen encoded by the nucleic acid sequence of the MVA vector is a first antigen, and the polypeptide antigen is a second antigen.

The first and second antigens may be different. In one embodiment, the first antigen is distinct from the second antigen. In one embodiment, the first and second antigens are the same.

In one embodiment, the polypeptide antigen is the same as the antigen encoded by the nucleic acid sequence. Thus, in one embodiment, the MVA vector comprises a nucleic acid sequence encoding an antigen that is the same as the polypeptide antigen.

In one embodiment, administration of the composition to a subject can elicit a combined T cell and antibody response against an antigen.

In one embodiment, the polypeptide antigen is a variant of the antigen encoded by the viral vector. In one embodiment, the polypeptide antigen is a fragment of the antigen encoded by the viral vector. Thus, in one embodiment, the polypeptide antigen comprises (or consists of) at least part of a polypeptide sequence of an antigen encoded by the nucleic acid sequence. In one embodiment wherein the polypeptide antigen is a fragment of the antigen encoded by the viral vector, administration of the composition to a subject can elicit a combined T cell and antibody response against said antigen.

In one embodiment, the composition further comprises an adenovirus vector, wherein said adenovirus vector comprises a nucleic acid sequence encoding an antigen. Suitable adenoviruses that may be used as adenovirus vectors in compositions comprising an MVA vector include, but are not limited to, human or simian adenoviruses, a group B adenovirus, a group C adenovirus, a group E adenovirus, adenovirus 6, PanAd3, adenovirus C3, AdCh63, Y25, AdC68, and Ad5.

In one embodiment, the adenovirus vector comprises a nucleic acid sequence encoding an antigen wherein the antigen is the same as the antigen encoded by the nucleic acid sequence of the MVA vector.

In one embodiment, the adenovirus vector comprises a nucleic acid sequence encoding an antigen, wherein the antigen is a polypeptide.

In one embodiment wherein the composition further comprises a polypeptide antigen, the adenovirus vector comprises a nucleic acid sequence encoding an antigen wherein the antigen is the same as the polypeptide antigen.

In one embodiment, the antigen encoded by the nucleic acid sequence of the adenovirus is an antigen from a pathogenic organism. Examples of suitable antigens include, but are not limited to, a malaria antigen, a tuberculosis antigen, an influenza antigen, or an HIV antigen.

In one embodiment, the antigen encoded by the nucleic acid sequence of the adenovirus is a malaria antigen. Examples of suitable malaria antigens include, but are not limited to, an antigen based on merozoite surface protein 1 (MSP1), such as an antigen based on MSP1 from Plasmodium falciparum, for example PfM115, PfMSP1₁₅, PfMSP1₁₉, PfMSP1₃₃, and PfMSP1₄₂; Plasmodium yoelii MSP1; apical membrane antigen-1 (AMA1); ME.TRAP (the TRAP sequence of P. falciparum, attached to a multi-epitope (ME) string that expresses Pb9, an H2K(d)-restricted epitope (SYIPSAEKI) that is immunodominant in BALB/c mice); PfM128. Other examples of suitable antigens include antigens derived from derived from P. falciparum and/or P. vivax, for example wherein the antigen is selected from DBP, PvTRAP, PvMSP2, PvMSP4, PvMSP5, PvMSP6, PvMSP7, PvMSP8, PvMSP9, PvAMAI and RBP or fragment thereof. Other example antigens derived from P. falciparum include, PfEMP-I, Pfs 16 antigen, MSP-I, MSP-3, LSA-I, LSA-3, AMA-I and TRAP. Other Plasmodium antigens include P. falciparum EBA, GLURP, RAPI, RAP2, Sequestrin, PO32, STARP, SALSA, PfEXPI, Pfs25, Pfs28, PFS27/25, Pfs48/45, Pfs230 and their analogues in other Plasmodium spp. Antigens from the mosquito vector of malaria may also be used where it may be desirable to block transmission of malaria, e.g the APN1 antigen.

In one embodiment, the antigen is the antigen encoded by the nucleic acid sequence of any one of SEQ ID NOs: 1-6.

In one embodiment, the adjuvant comprising a saponin is ISCOM Matrix.

In one embodiment, the emulsion is selected from: Montanide ISA720, Montanide ISA206, Emulsigen, and Titermax.

In one embodiment, the emulsion is selected from: Montanide ISA720, Montanide ISA206, Emulsigen, Titermax, and MF59.

In one embodiment, the adjuvant is a saponin.

In one embodiment, the saponin is a Quill A fraction, for example QS21.

The MVA and adenovirus vectors as described above may further comprise a promoter sequence. Suitable promoters for MVA and adenovirus vectors are known in the art. An example of a promoter that may be used in an adenovirus vector is the CMV promoter.

Methods of producing MVA and adenovirus vectors, for example MVA and adenovirus vectors as described above, are known in the art.

By way of example, a method of making a viral vector (such as an MVA vector or an adenovirus vector) may comprise providing a nucleic acid, wherein the nucleic acid comprises a nucleic acid sequence encoding a viral vector (for example an MVA vector or an adenovirus vector as described above); transfecting a host cell with the nucleic acid; culturing the host cell under conditions suitable for the expression of the nucleic acid; and obtaining the viral vector from the host cell. The nucleic acid comprising a sequence encoding a viral vector (as described above) may be generated by the use of any technique for manipulating and generating recombinant nucleic acid known in the art. As used herein, “transfecting” may mean any non-viral method of introducing nucleic acid into a cell. The nucleic acid may be any nucleic acid suitable for transfecting a host cell. The nucleic acid may be a plasmid. The host cell may be any cell in which a viral vector (as described above) may be grown. The host cell may be selected from the group consisting of: a 293 cell, a CHO cell, a CCL81.1 cell, a Vero cell, a HELA cell, a Per.C6 cell, and a BHK cell. As used herein, “culturing the host cell under conditions suitable for the expression of the nucleic acid” means using any cell culture conditions and techniques known in the art which are suitable for the chosen host cell, and which enable the viral vector to be produced in the host cell. As used herein, “obtaining the viral vector”, means using any technique known in the art that is suitable for separating the viral vector from the host cell. Thus, the host cells may be lysed to release the viral vector. The viral vector may subsequently be isolated and purified using any suitable method or methods known in the art.

In one aspect, the invention provides a composition comprising (a) an adenovirus vector, wherein said adenovirus vector comprises a nucleic acid sequence encoding an antigen, and wherein the adenovirus is selected from: a group B adenovirus, a group C adenovirus, and a group E adenovirus; and (b) an adjuvant comprising a saponin, or an emulsion; wherein the group B adenovirus is not an adenovirus 35, the group C adenovirus is not an adenovirus 5 having an intact E3 gene region, and the group E adenovirus is not an adenovirus C7.

Thus, the group C adenovirus is not an adenovirus 5 (Ad5) that has an intact E3 gene region—in this context, “intact” means that the gene region is still functional in the virus; for example the gene region has not been deleted.

The present inventors have found that combining certain specific adjuvants with specific adenoviruses as vectors produces a composition that surprisingly can elicit an increased immunological response when administered to a subject.

In one embodiment, the composition comprises a non-replicating adenovirus vector.

In one embodiment, the group C adenovirus is selected from: adenovirus 6, PanAd3, adenovirus C3, and Ad5 wherein the Ad5 lacks functional E1 and E3 gene regions. In one embodiment, the Ad5 has gene deletions in both the E1 and E3 gene regions.

In one embodiment, the group E adenovirus is selected from: AdCh63, Y25, and AdC68.

In one embodiment, the adenovirus is not Ad5.

In one embodiment, the adenovirus is selected from: adenovirus 6, PanAd3, adenovirus C3, AdCh63, Y25 and AdC68.

In one embodiment, the adjuvant comprising a saponin is ISCOM Matrix.

In one aspect, the invention provides a composition comprising (a) an adenovirus vector, wherein said adenovirus vector comprises a nucleic acid sequence encoding an antigen, and wherein the adenovirus is Ad5; and (b) an adjuvant selected from: Montanide ISA 720, Emulsigen, and Titermax.

In one embodiment, the adjuvant is selected from: Montanide ISA720, Montanide ISA206, Emulsigen, Titermax, and MF59.

The adenovirus vector comprises a nucleic acid sequence encoding an antigen. Subject to the size constraints imposed by the adenovirus vector, the antigen encoded may be any antigen.

In one embodiment, the antigen encoded by the nucleic acid sequence is a polypeptide.

In one embodiment, the antigen encoded by the nucleic acid sequence is any antigen described above as being encoded by the nucleic acid sequence of the MVA vector. Thus, in one embodiment, the antigen is an antigen from a pathogenic organism. Examples of suitable antigens include, but are not limited to, a malaria antigen, a tuberculosis antigen, an influenza antigen or an HIV antigen.

In one embodiment, the antigen encoded by the nucleic acid sequence is a malaria antigen, for example an antigen based on merozoite surface protein 1 (MSP1). In one embodiment, the antigen encoded by the viral vector is based on MSP1 from Plasmodium falciparum, for example PfM115, PfMSP1₁₅, PfMSP1₁₉, PfMSP1₃₃, and PfMSP1₄₂. In one embodiment, the antigen is Plasmodium yoelii MSP1. Further, non-limiting, examples of suitable malaria antigens include apical membrane antigen-1 (AMA1); ME.TRAP (the TRAP sequence of P. falciparum, attached to a multi-epitope (ME) string that expresses Pb9, an H2K(d)-restricted epitope (SYIPSAEKI) that is immunodominant in BALB/c mice); PfM128. Other examples of suitable antigens include antigens derived from antigens derived from P. falciparum and/or P. vivax, for example wherein the antigen is selected from DBP, PvTRAP, PvMSP2, PvMSP4, PvMSP5, PvMSP6, PvMSP7, PvMSP8, PvMSP9, PvAMAI and RBP or fragment thereof. Other example antigens derived from P. falciparum include, PfEMP-I, Pfs 16 antigen, MSP-I, MSP-3, LSA-I, LSA-3, AMA-I and TRAP. Other Plasmodium antigens include P. falciparum EBA, GLURP, RAPI, RAP2, Sequestrin, PO32, STARP, SALSA, PfEXPI, Pfs25, Pfs28, PFS27/25, Pfs48/45, Pfs230 and their analogues in other Plasmodium spp.

In one embodiment, a composition comprising an adenovirus vector (as described above) does not comprise a TLR ligand. Thus, in one embodiment, the composition is formulated as described above but lacks the presence of any additional component able to bind to and stimulate a TLR receptor.

In one embodiment, the composition when administered to a subject does not stimulate a TLR-mediated response.

In one embodiment, a composition comprising an adenovirus vector (as described above) further comprises a polypeptide antigen. In one embodiment, the presence of a polypeptide antigen means that, following administration of the composition to a subject, a simultaneous T cell and antibody response may be achieved. In one embodiment, the T cell and antibody response achieved surpasses that achieved when either a viral vector or polypeptide antigen are used alone.

In one embodiment, the polypeptide antigen is a polypeptide antigen from a pathogenic organism. Examples of suitable antigens include, but are not limited to, a malaria polypeptide antigen, a tuberculosis polypeptide antigen, an influenza polypeptide antigen, or an HIV polypeptide antigen.

In one embodiment, the polypeptide antigen is a malaria antigen. Examples of suitable malaria antigens include, but are not limited to, an antigen based on merozoite surface protein 1 (MSP1), such as an antigen based on MSP1 from Plasmodium falciparum, for example PfM115, PfMSP1₁₅, PfMSP1₁₉, PfMSP1₃₃, and PfMSP1₄₂; Plasmodium yoelii MSP1; apical membrane antigen-1 (AMA1); ME.TRAP (the TRAP sequence of P. falciparum, attached to a multi-epitope (ME) string that expresses Pb9, an H2K(d)-restricted epitope (SYIPSAEKI) that is immunodominant in BALB/c mice); PfM128. Other suitable antigens also include antigens derived from P. falciparum and/or P. vivax, for example wherein the antigen is selected from DBP, PvTRAP, PvMSP2, PvMSP4, PvMSP5, PvMSP6, PvMSP7, PvMSP8, PvMSP9, PvAMAI and RBP or fragment thereof. Other example antigens derived from P. falciparum include, PfEMP-I, Pfs 16 antigen, MSP-I, MSP-3, LSA-I, LSA-3, AMA-I and TRAP. Other Plasmodium antigens include P. falciparum EBA, GLURP, RAPI, RAP2, Sequestrin, PO32, STARP, SALSA, PfEXPI, Pfs25, Pfs28, PFS27/25, Pfs48/45, Pfs230 and their analogues in other Plasmodium spp. Antigens from the mosquito vector of malaria may also be used where it may be desirable to block transmission of malaria, e.g the APN1 antigen.

In one embodiment, the polypeptide antigen is the antigen encoded by the nucleic acid sequence of any one of SEQ ID NOs: 1-6.

In one embodiment, the polypeptide antigen is an antigen from a pathogenic organism.

In one embodiment, the polypeptide antigen is not covalently bonded to the adenovirus vector. In one embodiment, the polypeptide antigen is a separate component to the adenovirus vector.

In one embodiment, the antigen encoded by the nucleic acid sequence of the adenovirus vector is a first antigen, and the polypeptide antigen is a second antigen.

The first and second antigens may be different. In one embodiment, the first antigen is distinct from the second antigen. In one embodiment, the first and second antigens are the same.

In one embodiment, the polypeptide antigen is the same as the antigen encoded by the nucleic acid sequence. Thus, in one embodiment, the adenovirus vector comprises a nucleic acid sequence encoding an antigen that is the same as the polypeptide antigen.

In one embodiment, the composition further comprises a polypeptide antigen wherein the polypeptide antigen is different from the antigen encoded by the nucleic acid sequence.

In one embodiment, administration of the composition to a subject can elicit a simultaneous immune response against different antigens.

In one embodiment, the polypeptide antigen is the same as the antigen encoded by the nucleic acid sequence. Thus, in one embodiment, the adenovirus vector comprises a nucleic acid sequence encoding an antigen that is the same as the polypeptide antigen.

In one embodiment, administration of the composition to a subject can elicit a combined T cell and antibody response against an antigen.

In one embodiment, the polypeptide antigen is a variant of the antigen encoded by the viral vector. In one embodiment, the polypeptide antigen is a fragment of the antigen encoded by the viral vector. Thus, in one embodiment, the polypeptide antigen comprises (or consists of) at least part of a polypeptide sequence of an antigen encoded by the nucleic acid sequence.

In one aspect, the invention provides a composition (as described above) for use in medicine.

In one aspect, the invention provides a composition (as described above) for use in stimulating or inducing an immune response in a subject. In one embodiment, stimulating or inducing an immune response in a subject comprises administering to the subject a composition (as described above). In one embodiment, stimulating or inducing an immune response in a subject comprises administering to the subject a composition (as described above) wherein the composition is sequentially administered multiple times (for example, wherein the composition is administered two, three or four times). Thus, in one embodiment, the subject is administered a composition (as described above) and is then administered the same composition (or a substantially similar composition) again at a different time.

In one embodiment, stimulating or inducing an immune response in a subject comprises administering a composition (as described above) to a subject, wherein said composition is administered substantially prior to, simultaneously with or subsequent to another immunogenic composition.

Prior, simultaneous and sequential administration regimes are discussed in more detail below.

In one aspect, the invention provides a composition (as described above) for use in the prevention or treatment of an infectious disease. Non-limiting examples of infectious diseases that may be prevented or treated include malaria, tuberculosis, influenza, and HIV/AIDS.

In one embodiment, the infectious disease is selected from the group consisting of diseases caused by: Plasmodia, influenza viruses, Mycobacterium tuberculosis, Mycobacterium bovis, other Mycobacteria, hepatitis C virus, other flaviviruses, hepatitis B virus, human immunodeficiency virus, other retroviruses, Staphylococcus aureus, other Staphylococci, Streptococcus pneumoniae, Streptococcus pyogenes, other Streptococci, Haemophilus influenzae, Neisseria meningitides.

In one embodiment, the infectious disease is not a disease caused by a Chlamydia sp. (e.g. C. trachomatis or C. pneumoniae) infection.

In one embodiment, the disease to be prevented or treated is a human disease, and the subject to be is a human. In one embodiment, the disease to be prevented or treated is a disease of a (non-human) animal, and the subject is a (non-human) animal.

The composition of the present invention may be useful for inducing a range of immune responses and may therefore be useful in methods for treating a range of diseases.

As used herein, the term “treatment” or “treating” embraces therapeutic or preventative/prophylactic measures, and includes post-infection therapy and amelioration of an infectious disease.

As used herein, the term “preventing” includes preventing the initiation of an infectious disease and/or reducing the severity or intensity of an infectious disease.

A composition of the invention (as described above) may be administered to a subject (typically a mammalian subject such as a human, bovine, porcine, ovine, caprine, equine, cervine, canine or feline subject) already having an infectious disease, to treat or prevent said infectious disease. In one embodiment, the subject is suspected of having come into contact with an infectious disease (or the disease-causing agent), or has had known contact with an infectious disease (or the disease-causing agent), but is not yet showing symptoms of exposure to said infectious disease (or said disease-causing agent).

When administered to a subject (e.g. a mammal such as a human, bovine, porcine, ovine, caprine, equine, cervine, canine or feline subject) that already has an infectious disease, or is showing symptoms associated with an infectious disease, a composition of the invention (as described above) can cure, delay, reduce the severity of, or ameliorate one or more symptoms of, the infectious disease; and/or prolong the survival of a subject beyond that expected in the absence of such treatment.

Alternatively, a composition of the invention (as described above) may be administered to a subject (e.g. a human, bovine, porcine, ovine, caprine, equine, cervine, canine or feline subject) who may ultimately contract an infectious disease, in order to prevent, cure, delay, reduce the severity of, or ameliorate one or more symptoms of, said infectious disease; or in order to prolong the survival of a subject beyond that expected in the absence of such treatment.

In one embodiment, the subject has previously been exposed to an infectious disease. For example, the subject may have had an infectious disease in the past (but is optionally not currently infected with the disease-causing agent of the infectious disease). The subject may be latently infected with an infectious disease. Alternatively, or in addition, the subject may have been vaccinated against said infectious disease in the past.

The treatments and preventative therapies in which compositions of the present invention may be used are applicable to a variety of different subjects of different ages. In the context of humans, the therapies are applicable to children (e.g. infants, children under 5 years old, older children or teenagers) and adults. In the context of other animal subjects (e.g. mammals such as bovine, porcine or equine subjects), the therapies are applicable to immature subjects (e.g. calves, piglets, foals) and mature/adult subjects. The treatments and preventative therapies of the present invention are applicable to subjects who are immunocompromised or immunosuppressed (e.g. human patients who have HIV or AIDS, or other animal patients with comparable immunodeficiency diseases), subjects who have undergone an organ transplant, bone marrow transplant, or who have genetic immunodeficiencies.

The compositions of the invention (as described above) can be employed as vaccines.

As used, herein, a “vaccine” is a formulation that, when administered to an animal subject such as a mammal (e.g. a human, bovine, porcine, ovine, caprine, equine, cervine, canine or feline subject) stimulates a protective immune response against an infectious disease. The immune response may be a humoral and/or a cell-mediated immune response. Thus, the vaccine may stimulate B-cells and/or T-cells. A vaccine of the invention can be used, for example, to protect an animal from the effects of an infectious disease (for example, malaria, influenza or tuberculosis).

The term “vaccine” is herein used interchangeably with the terms “therapeutic/prophylactic composition”, “formulation”, “antigenic composition”, or “medicament”.

In one aspect, the invention provides a vaccine composition, comprising a composition (as described above); and a pharmaceutically acceptable carrier.

The vaccine of the invention (as defined above) in addition to a pharmaceutically acceptable carrier can further be combined with one or more of a salt, excipient, diluent, adjuvant, immunoregulatory agent and/or antimicrobial compound.

In one aspect, the invention provides an immunological composition, comprising a composition (as described above); and a pharmaceutically acceptable carrier.

The immunological composition in addition to a pharmaceutically acceptable carrier can further be combined with one or more of a salt, excipient, diluent, adjuvant, immunoregulatory agent and/or antimicrobial compound.

In one aspect, the invention provides a pharmaceutical composition, comprising a composition (as described above); and a pharmaceutically acceptable carrier.

The pharmaceutical composition in addition to a pharmaceutically acceptable carrier can further be combined with one or more of a salt, excipient, diluent, adjuvant, immunoregulatory agent and/or antimicrobial compound.

The composition may be formulated into a vaccine, immunogenic composition or pharmaceutical composition as neutral or salt forms. Pharmaceutically acceptable salts include acid addition salts formed with inorganic acids such as, for example, hydrochloric or phosphoric acids, or with organic acids such as acetic, oxalic, tartaric, maleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In one aspect, the invention provides an MVA vector comprising a nucleic acid sequence encoding an antigen for use in a method of stimulating or inducing an immune response in a subject, or for use in a method of preventing or treating an infectious disease, wherein the method further comprises administration of a polypeptide antigen, and wherein either one or both of the MVA vector and the polypeptide antigen is administered in combination with an adjuvant comprising a saponin, or an emulsion.

The antigen encoded by the nucleic acid may be any suitable antigen as described above. The polypeptide antigen may be any suitable polypeptide antigen as described above.

In one embodiment, the adjuvant comprises a saponin.

In one embodiment, the adjuvant is an emulsion.

In one embodiment, the adjuvant comprising a saponin is ISCOM Matrix.

In one embodiment, the polypeptide antigen is an antigen from a pathogenic organism.

In one embodiment, the antigen encoded by the nucleic acid sequence of the MVA vector and the polypeptide antigen are the same.

In one embodiment, the polypeptide antigen comprises a variant of the antigen encoded by the nucleic acid sequence of the MVA vector. In one embodiment, the polypeptide antigen comprises a fragment of the antigen encoded by the nucleic acid sequence of the MVA vector. In one embodiment, the antigen encoded by the nucleic acid sequence of the MVA vector and the polypeptide antigen are different. The adjuvant may be administered together with the MVA vector, together with the polypeptide antigen, or together with both.

In one embodiment, the MVA vector and the polypeptide antigen are administered to the subject sequentially, in either order.

In one embodiment wherein the MVA vector and the polypeptide antigen are administered together with an adjuvant, the adjuvant administered with the MVA vector is the same as the adjuvant administered with the polypeptide antigen.

“Administered to the subject sequentially” has the meaning of “sequential administration” as defined below. Thus, the MVA vector and the polypeptide antigen are administered at (substantially) different times, one after the other. Such sequential administration may form part of a prime-boost regime. In one embodiment, the MVA vector is administered first, and the polypeptide antigen administered second. In one embodiment, the polypeptide antigen is administered first, and the MVA vector administered second.

In one embodiment, the method further comprises administration of an adenovirus vector, wherein said adenovirus vector comprises a nucleic acid sequence encoding an antigen. The adenovirus vector may be administered in combination with an adjuvant comprising a saponin, or an emulsion. In one embodiment, the adjuvant comprising a saponin is ISCOM Matrix. The antigen encoded by the nucleic acid sequence of the adenovirus vector may be any suitable antigen as described above.

Thus, in one embodiment, the subject is administered an adenovirus vector in addition to being administered an MVA vector and a polypeptide antigen (as described above).

Suitable adenoviruses that may be used as adenovirus vectors include, but are not limited to, human or simian adenoviruses, a group B adenovirus, a group C adenovirus, a group E adenovirus, adenovirus 6, PanAd3, adenovirus C3, AdCh63, Y25, AdC68, and Ad5.

In one embodiment, the adenovirus vector is administered with an adjuvant wherein the adjuvant is the same as an adjuvant administered with one or both of the MVA vector and the polypeptide antigen.

In one embodiment, the MVA vector, the polypeptide antigen and the adenovirus vector are administered to the subject sequentially, in any order. Thus, in one embodiment, the subject may be administered sequentially the MVA vector (“M”), the polypeptide antigen (“P”), and the adenovirus vector (“A”) in any one of the following orders: A-M-P, A-P-M, M-A-P, M-P-A, P-M-A, P-A-M. As described above, adjuvant may be administered with one, two or all three of the MVA vector, the polypeptide antigen, and the adenovirus vector.

In one embodiment, the adenovirus vector is administered to the subject in combination with either the MVA vector or the polypeptide antigen.

In one embodiment, the method comprises sequential administration of (a) a combination of the MVA vector and the polypeptide antigen, and (b) the adenovirus vector, in either order.

In one embodiment, the adenovirus vector is administered in combination with a polypeptide antigen.

When two of the components described above (the MVA vector, the polypeptide antigen, and the adenovirus vector) are administered in combination, this means that they are administered at (substantially) the same time, for example simultaneously.

Thus, in one embodiment, the subject may be administered sequentially the MVA vector (“M”), the polypeptide antigen (“P”), and the adenovirus vector (“A”) in any one of the following orders, where brackets denote a combination: (A+M)-P, P-(A+M), (A+P)-M, M-(A+P), (M+P)-A, A-(M+P), (A+P)-(M+P), (M+P)-(A+P).

Any of the above administration orders may be applied as part of a prime-boost protocol.

In one aspect, the invention provides an MVA vector comprising a nucleic acid sequence encoding an antigen for use in a method of stimulating or inducing an immune response in a subject, or for use in a method of preventing or treating an infectious disease, wherein the method further comprises administration of an adenovirus vector, wherein said adenovirus vector comprises a nucleic acid sequence encoding an antigen; and wherein either one or both of the MVA vector and the adenovirus vector is administered in combination with an adjuvant comprising a saponin, or an emulsion. In one embodiment, the adjuvant comprising a saponin is ISCOM Matrix.

The antigen encoded by the nucleic acid of the MVA vector may be any suitable antigen as described above. The antigen encoded by the nucleic acid of the adenovirus vector may be any suitable antigen as described above.

In one embodiment, the MVA vector and the adenovirus vector are administered to the subject sequentially, in either order. Thus, in one embodiment, the MVA vector (“M”) and the adenovirus (“A”) are administered in the order M-A, or in the order A-M. Either one or both of the MVA vector and the adenovirus vector may be administered in combination with the adjuvant.

In one aspect, the invention provides an adenovirus vector comprising a nucleic acid sequence encoding an antigen for use in a method of stimulating or inducing an immune response in a subject, or for use in a method of preventing or treating an infectious disease, wherein the method further comprises administration of a polypeptide antigen, and wherein either one or both of the adenovirus vector and the polypeptide is administered in combination with an adjuvant comprising a saponin, an emulsion, or an alum adjuvant. In one embodiment, the adjuvant comprising a saponin is ISCOM Matrix. In one embodiment, the adjuvant is an alum adjuvant.

The antigen encoded by the nucleic acid of the adenovirus vector may be any suitable antigen as described above. The polypeptide antigen may be any suitable polypeptide antigen as described above.

In one embodiment, the adenovirus is selected from adenovirus 6, PanAd3, adenovirus C3, AdCh63, Y25, AcC68, and Ad5 wherein the Ad5 has gene deletions in both the E1 and E3 gene regions.

In one embodiment, the adenovirus is not Ad5.

In one embodiment, the adenovirus is selected from: adenovirus 6, PanAd3, adenovirus C3, AdCh63, Y25 and AdC68.

In one embodiment, the adenovirus vector and the polypeptide antigen are administered to the subject sequentially, in either order. Thus, in one embodiment, the adenovirus vector (“A”) and the polypeptide antigen (“P”) may be administered in the order A-P, or in the order P-A.

In one aspect, the invention provides a kit for use in medicine comprising: (a) an adenovirus vector, wherein said adenovirus vector comprises a nucleic acid sequence encoding an antigen; and/or an MVA vector, wherein said MVA vector comprises a nucleic acid sequence encoding an antigen; (b) a polypeptide antigen; and (d) an adjuvant comprising a saponin, or an emulsion. In one embodiment, the adjuvant comprising a saponin is ISCOM Matrix.

Administration of immunogenic compositions, therapeutic formulations, medicaments, pharmaceutical compositions, and prophylactic formulations (e.g. vaccines) is generally by conventional routes e.g. intravenous, subcutaneous, intraperitoneal, or mucosal routes. The administration may be by parenteral administration; for example, a subcutaneous or intramuscular injection.

Accordingly, immunogenic compositions, therapeutic formulations, medicaments, pharmaceutical compositions, and prophylactic formulations (e.g. vaccines) of the invention may be prepared as injectables, either as liquid solutions or suspensions. Solid forms suitable for solution in, or suspension in, liquid prior to injection may alternatively be prepared. The preparation may also be emulsified, or the peptide encapsulated in liposomes or microcapsules.

The active ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the immunogenic compositions, therapeutic formulations, medicaments, pharmaceutical compositions, and prophylactic formulations (e.g. vaccines) may contain minor amounts of auxiliary substances such as wetting or emulsifying agents, and/or pH buffering agents.

Generally, the carrier is a pharmaceutically-acceptable carrier. Non-limiting examples of pharmaceutically acceptable carriers include water, saline, and phosphate-buffered saline. In some embodiments, however, the composition is in lyophilized form, in which case it may include a stabilizer, such as bovine serum albumin (BSA). In some embodiments, it may be desirable to formulate the composition with a preservative, such as thiomersal or sodium azide, to facilitate long term storage.

Examples of buffering agents include, but are not limited to, sodium succinate (pH 6.5), and phosphate buffered saline (PBS; pH 6.5 and 7.5).

Additional formulations which are suitable for other modes of administration include suppositories and, in some cases, oral formulations or formulations suitable for distribution as aerosols. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

It may be desired to direct the compositions of the present invention (as described above) to the respiratory system of a subject; for example, for use in the treatment or prevention of a respiratory disease. Efficient transmission of a therapeutic/prophylactic composition or medicament to the site of infection in the lungs may be achieved by oral or intra-nasal administration.

Formulations for intranasal administration may be in the form of nasal droplets or a nasal spray. An intranasal formulation may comprise droplets having approximate diameters in the range of 100-5000 μm, such as 500-4000 μm, 1000-3000 μm or 100-1000 μm. Alternatively, in terms of volume, the droplets may be in the range of about 0.001-100 μl, such as 0.1-50 μl or 1.0-25 μl, or such as 0.001-1 μl.

Alternatively, the therapeutic/prophylactic formulation or medicament may be an aerosol formulation. The aerosol formulation may take the form of a powder, suspension or solution. The size of aerosol particles is relevant to the delivery capability of an aerosol. Smaller particles may travel further down the respiratory airway towards the alveoli than would larger particles. In one embodiment, the aerosol particles have a diameter distribution to facilitate delivery along the entire length of the bronchi, bronchioles, and alveoli. Alternatively, the particle size distribution may be selected to target a particular section of the respiratory airway, for example the alveoli. In the case of aerosol delivery of the medicament, the particles may have diameters in the approximate range of 0.1-50 μm, preferably 1-25 μm, more preferably 1-5 μm.

Aerosol particles may be for delivery using a nebulizer (e.g. via the mouth) or nasal spray. An aerosol formulation may optionally contain a propellant and/or surfactant.

By controlling the size of the droplets/particles to within the defined range of the present invention, it is possible to avoid (or minimize) inadvertent medicament delivery to the alveoli and thus avoid alveoli-associated pathological problems such as inflammation and fibrotic scarring of the lungs.

Intra-nasal vaccination engages both T- and B-cell mediated effector mechanisms in nasal and bronchus associated mucosal tissues, which differ from other mucosa-associated lymphoid tissues. The protective mechanisms invoked by the intranasal route of administration may include: the activation of T-lymphocytes with preferential lung homing; up-regulation of co-stimulatory molecules (e.g. B7.2); and/or activation of macrophages or secretory IgA antibodies.

Intranasal delivery of compositions of the invention (as described above) may facilitate the invoking of a mucosal antibody response, which is favoured by a shift in the T-cell response toward the Th2 phenotype which helps antibody production. A mucosal response is characterised by enhanced IgA production, and a Th2 response is characterised by enhanced IL-4 production.

In one embodiment, the immunogenic compositions, therapeutic formulations, medicaments, pharmaceutical compositions, and prophylactic formulations (e.g. vaccines) of the invention comprise a pharmaceutically acceptable carrier, and optionally one or more of a salt, excipient, diluent and/or adjuvant.

In one embodiment, the immunogenic compositions, therapeutic formulations, medicaments, pharmaceutical compositions, and prophylactic formulations (e.g. vaccines) of the invention may comprise one or more immunoregulatory agents selected from, for example, immunoglobulins, antibiotics, interleukins (e.g. IL-2, IL-12), and/or cytokines (e.g. IFNγ).

In one embodiment, the immunogenic compositions, therapeutic formulations, medicaments, pharmaceutical compositions, and prophylactic formulations (e.g. vaccines) of the invention may comprise one or more antimicrobial compounds, (for example, conventional anti-tuberculosis drugs such as rifampicin, isoniazid, ethambutol or pyrizinamide).

The immunogenic compositions, therapeutic formulations, medicaments, pharmaceutical compositions, and prophylactic formulations (e.g. vaccines) of the invention may be given in a single dose schedule (i.e. the full dose is given at substantially one time). Alternatively, the immunogenic compositions, therapeutic formulations, medicaments, pharmaceutical compositions, and prophylactic formulations (e.g. vaccines) of the invention may be given in a multiple dose schedule.

A multiple dose schedule is one in which a primary course of treatment (e.g. vaccination) may be with 1-6 separate doses, followed by other doses given at subsequent time intervals required to maintain and or reinforce the immune response, for example (for human subjects), at 1-4 months for a second dose, and if needed, a subsequent dose(s) after a further 1-4 months.

The dosage regimen will be determined, at least in part, by the need of the individual and be dependent upon the judgment of the practitioner (e.g. doctor or veterinarian).

Simultaneous administration means administration at (substantially) the same time.

Sequential administration of two or more compositions/therapeutic agents/vaccines means that the compositions/therapeutic agents/vaccines are administered at (substantially) different times, one after the other.

For example, sequential administration may encompass administration of two or more compositions/therapeutic agents/vaccines at different times, wherein the different times are separated by a number of days (for example, 1, 2, 5, 10, 15, 20, 30, 60, 90, 100, 150 or 200 days).

For example, in one embodiment, the vaccine of the present invention may be administered as part of a ‘prime-boost’ vaccination regime.

In one embodiment, the immunogenic compositions, therapeutic formulations, medicaments, pharmaceutical compositions, and prophylactic formulations (e.g. vaccines) of the invention can be administered to a subject such as a mammal (e.g. a human, bovine, porcine, ovine, caprine, equine, cervine, canine or feline subject) in conjunction with (simultaneously or sequentially) one or more immunoregulatory agents selected from, for example, immunoglobulins, antibiotics, interleukins (e.g. IL-2, IL-12), and/or cytokines (e.g. IFNγ).

In one embodiment, the immunogenic compositions, therapeutic formulations, medicaments, pharmaceutical compositions, and prophylactic formulations (e.g. vaccines) of the invention can be administered to a subject such as a mammal (e.g. a human, bovine, porcine, ovine, caprine, equine, cervine, canine or feline subject) in conjunction with (simultaneously or sequentially) one or more antimicrobial compounds, such as conventional anti-tuberculosis drugs (e.g. rifampicin, isoniazid, ethambutol or pyrizinamide).

The immunogenic compositions, therapeutic formulations, medicaments, pharmaceutical compositions, and prophylactic formulations (e.g. vaccines) may contain 5% to 95% of active ingredient, such as at least 10% or 25% of active ingredient, or at least 40% of active ingredient or at least 50, 55, 60, 70 or 75% active ingredient.

The immunogenic compositions, therapeutic formulations, medicaments, pharmaceutical compositions, and prophylactic formulations (e.g. vaccines) are administered in a manner compatible with the dosage formulation, and in such amount as will be prophylactically and/or therapeutically effective.

In this regard, as used herein, an “effective amount” is a dosage or amount that is sufficient to achieve a desired biological outcome. As used herein, a “therapeutically effective amount” is an amount which is effective, upon single or multiple dose administration to a subject (such as a mammal—e.g. a human, bovine, porcine, ovine, caprine, equine, cervine, canine or feline subject) for treating, preventing, curing, delaying, reducing the severity of, ameliorating at least one symptom of a disorder or recurring disorder, or prolonging the survival of the subject beyond that expected in the absence of such treatment.

Accordingly, the quantity of active ingredient to be administered depends on the subject to be treated, capacity of the subject's immune system to generate a protective immune response, and the degree of protection desired. Precise amounts of active ingredient required to be administered may depend on the judgment of the practitioner and may be particular to each subject.

The present invention encompasses polypeptides that are substantially homologous to polypeptides based on any one of the polypeptide antigens identified in this application (including fragments thereof). The terms “sequence identity” and “sequence homology” are considered synonymous in this specification.

By way of example, a polypeptide of interest may comprise an amino acid sequence having at least 70, 75, 80, 82, 84, 86, 88, 90, 92, 94, 96, 98, 99 or 100% amino acid sequence identity with the amino acid sequence of a reference polypeptide.

There are many established algorithms available to align two amino acid sequences.

Typically, one sequence acts as a reference sequence, to which test sequences may be compared. The sequence comparison algorithm calculates the percentage sequence identity for the test sequence(s) relative to the reference sequence, based on the designated program parameters. Alignment of amino acid sequences for comparison may be conducted, for example, by computer implemented algorithms (e.g. GAP, BESTFIT, FASTA or TFASTA), or BLAST and BLAST 2.0 algorithms.

The BLOSUM62 table shown below is an amino acid substitution matrix derived from about 2,000 local multiple alignments of protein sequence segments, representing highly conserved regions of more than 500 groups of related proteins (Henikoff & Henikoff, Proc. Natl. Acad. Sci. USA 89:10915-10919, 1992; incorporated herein by reference). Amino acids are indicated by the standard one-letter codes. The percent identity is calculated as:

$$\frac{Total number of identical matches}{[ \begin{matrix}\begin{matrix}length of the longer sequence plus the number of gaps \\ Introduced into the longer sequence in order to align\end{matrix} \\ the two sequences\end{matrix} ]} \times 100$$

BLOSUM62 table

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

In a homology comparison, the identity may exist over a region of the sequences that is at least 10 amino acid residues in length (e.g. at least 15, 20, 30, 40, 50, 75, 100, 150, 200, 250, 300, 350, 400, 450, 500, 550, 600, 650 or 685 amino acid residues in length—e.g. up to the entire length of the reference sequence.

Substantially homologous polypeptides have one or more amino acid substitutions, deletions, or additions. In many embodiments, those changes are of a minor nature, for example, involving only conservative amino acid substitutions. Conservative substitutions are those made by replacing one amino acid with another amino acid within the following groups: Basic: arginine, lysine, histidine; Acidic: glutamic acid, aspartic acid; Polar: glutamine, asparagine; Hydrophobic: leucine, isoleucine, valine; Aromatic: phenylalanine, tryptophan, tyrosine; Small: glycine, alanine, serine, threonine, methionine. Substantially homologous polypeptides also encompass those comprising other substitutions that do not significantly affect the folding or activity of the polypeptide; small deletions, typically of 1 to about 30 amino acids (such as 1-10, or 1-5 amino acids); and small amino- or carboxyl-terminal extensions, such as an amino-terminal methionine residue, a small linker peptide of up to about 20-25 residues, or an affinity tag.

The polypeptides of the invention may also comprise non-naturally occurring amino acid residues. In this regard, in addition to the 20 standard amino acids, non-standard amino acids (such as 4-hydroxyproline, 6-N-methyl lysine, 2-aminoisobutyric acid, isovaline and α-methyl serine) may be substituted for amino acid residues of the mycobacterial polypeptides of the present invention. A limited number of non-conservative amino acids, amino acids that are not encoded by the genetic code, and unnatural amino acids may be substituted for mycobacterial polypeptide amino acid residues. Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethyl homo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine.

Several methods are known in the art for incorporating non-naturally occurring amino acid residues into polypeptides. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations can be carried out in a cell free system comprising an E. coli S30 extract and commercially available enzymes and other reagents. Peptides can be, for instance, purified by chromatography. In a second method, translation is carried out in Xenopus oocytes by microinjection of mutated mRNA and chemically aminoacylated suppressor tRNAs. Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. Naturally occurring amino acid residues can be converted to non-naturally occurring species by in vitro chemical modification. Chemical modification can be combined with site-directed mutagenesis to further expand the range of substitutions.

Essential amino acids, such as those in the polypeptides of the present invention, can be identified according to procedures known in the art, such as site-directed mutagenesis or alanine-scanning mutagenesis. Sites of biological interaction can also be determined by physical analysis of structure, as determined by such techniques as nuclear magnetic resonance, crystallography, electron diffraction or photoaffinity labeling, in conjunction with mutation of putative contact site amino acids. The identities of essential amino acids can also be inferred from analysis of homologies with related family members of the polypeptide of interest.

Multiple amino acid substitutions can be made and tested using known methods of mutagenesis and screening. Methods are known for simultaneously randomizing two or more positions in a polypeptide, selecting for functional polypeptide, and then sequencing the mutagenized polypeptides to determine the spectrum of allowable substitutions at each position. Other methods that can be used include phage display.

Routine deletion analyses of nucleic acid molecules can be performed to obtain functional fragments of a nucleic acid molecule that encodes a polypeptide of the invention. As an illustration, DNA molecules can be digested with Bal31 nuclease to obtain a series of nested deletions. These DNA fragments are then inserted into expression vectors in proper reading frame, and the expressed polypeptides are isolated and tested for the desired activity. An alternative to exonuclease digestion is to use oligonucleotide-directed mutagenesis to introduce deletions, or stop codons to specify production of a desired fragment. Alternatively, particular polynucleotide fragments can be synthesized using the polymerase chain reaction.

A mutant of a polypeptide of the invention may contain one or more analogues of an amino acid (e.g. an unnatural amino acid), or a substituted linkage, as compared with the sequence of the reference polypeptide. In a further embodiment, a polypeptide of interest may be a mimic of the reference polypeptide, which mimic reproduces at least one epitope of the reference polypeptide.

Mutants of the disclosed polynucleotide and polypeptide sequences of the invention can be generated through DNA shuffling. Briefly, mutant DNAs are generated by in vitro homologous recombination by random fragmentation of a parent DNA followed by reassembly using PCR, resulting in randomly introduced point mutations. This technique can be modified by using a family of parent DNAs, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed above can be combined with high-throughput screening methods to detect activity of cloned mutant polypeptides. Mutagenized nucleic acid molecules that encode polypeptides of the invention, or fragments thereof, can be recovered from the host cells and rapidly sequenced using modern equipment. These methods allow the rapid determination of the importance of individual amino acid residues in a polypeptide of interest, and can be applied to polypeptides of unknown structure.

A “fragment” of a polypeptide of interest comprises a series of consecutive amino acid residues from the sequence of said polypeptide. By way of example, a “fragment” of a polypeptide of interest may comprise (or consist of) at least 10 consecutive amino acid residues from the sequence of said polypeptide (e.g. at least 15, 20, 25, 28, 30, 35, 40, 45, 50, 55, 75, 100, 125, 150, 175, 200, 225, 250, 275, 300, 325, 350, 375, 400 or 412 consecutive amino acid residues of said polypeptide). A fragment may include at least one epitope of the polypeptide of interest.

A polypeptide of interest, or fragment, may possess the active site of the reference polypeptide.

The polypeptide of interest, or fragment thereof, may have a common antigenic cross-reactivity and/or substantially the same in vivo biological activity as the reference peptide. For example, the polypeptides, or polypeptide fragments, and reference polypeptides share a common ability to induce a “recall response” of a T-lymphocyte (e.g. CD4+, CD8+, effector T cell or memory T cell such as a TEM or TCM), which has been previously exposed to an antigenic component of a mycobacterial infection.

New immunological assays for measuring and quantifying T cell responses have been established over the last 10 years. For example, the interferon-gamma (IFN-γ) ELISPOT assay is useful as an immunological readout because the secretion of IFN-γ from antigen-specific T cells is a good correlate of protection against M. tuberculosis. Furthermore, the ELISPOT assay is a very reproducible and sensitive method of quantifying the number of IFN-γ secreting antigen-specific T cells.

As used herein, the terms “nucleic acid sequence” and “polynucleotide” are used interchangeably and do not imply any length restriction. As used herein, the terms “nucleic acid” and “nucleotide” are used interchangeably. The terms “nucleic acid sequence” and “polynucleotide” embrace DNA (including cDNA) and RNA sequences.

The polynucleotide sequences of the present invention include nucleic acid sequences that have been removed from their naturally occurring environment, recombinant or cloned DNA isolates, and chemically synthesized analogues or analogues biologically synthesized by heterologous systems.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The polynucleotides of the present invention may also be produced by chemical synthesis, e.g. by the phosphoramidite method or the triester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

When applied to a nucleic acid sequence, the term “isolated” in the context of the present invention denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In view of the degeneracy of the genetic code, considerable sequence variation is possible among the polynucleotides of the present invention. Degenerate codons encompassing all possible codons for a given amino acid are set forth below:

| Amino | | Degenerate |
|---|---|---|
| Acid | Codons | Codon |
| Cys | TGC TGT | TGY |
| Ser | AGC AGT TCA TCC TCG TCT | WSN |
| Thr | ACA ACC ACG ACT | ACN |
| Pro | CCA CCC CCG CCT | CCN |
| Ala | GCA GCC GCG GCT | GCN |
| Gly | GGA GGC GGG GGT | GGN |
| Asn | AAC AAT | AAY |
| Asp | GAC GAT | GAY |
| Glu | GAA GAG | GAR |
| Gln | CAA CAG | CAR |
| His | CAC CAT | CAY |
| Arg | AGA AGG CGA CGC CGG CGT | MGN |
| Lys | AAA AAG | AAR |
| Met | ATG | ATG |
| Ile | ATA ATC ATT | ATH |
| Leu | CTA CTC CTG CTT TTA TTG | YTN |
| Val | GTA GTC GTG GTT | GTN |
| Phe | TTC TTT | TTY |
| Tyr | TAC TAT | TAY |
| Trp | TGG | TGG |
| Ter | TAA TAG TGA | TRR |
| Asn/Asp | | RAY |
| Glu/Gin | | SAR |
| Any | | NNN |

One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 82, 84, 86, 88, 90, 92, 94, 96, 98 or 99% of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different Thr codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species.

Thus, in one embodiment of the invention, the nucleic acid sequence is codon optimized for expression in a host cell.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). A fragment may include at least one antigenic determinant and/or may encode at least one antigenic epitope of the corresponding polypeptide of interest.

Key to SEQ ID NOs

SEQ ID NO: 1 TPA-PfAMA1 (3D7)-gene with tpa leader.

SEQ ID NO: 2 TPA-PyMSP1₄₂-PK-gene with tpa leader and PK tag.

SEQ ID NO: 3 PfMSP1₁₅.

SEQ ID NO: 4 GFP.

SEQ ID NO: 5 ME-TRAP.

SEQ ID NO: 6 PfM128.

SEQ ID NO: 7 AdHu5 genome.

SEQ ID NO: 8 AdCh63 genome.

TPA-PfAMA1 (3D7) - gene with tpa leader.
SEQ ID NO: 1
atgaagagagggctctgctgtgtgctgctgctgtgtggagcagtcttcgtttcgcccagccaggaaatccatgccc
gattcagaagactcgacCAGAACTACTGGGAGCACCCTTACCAGAACAGCGACGTG
TATCGGCCCATCAACGAGCACAGAGAGCACCCCAAAGAATACGAGTATCCC
CTGCACCAGGAACACACCTACCAGCAGGAAGATAGCGGCGAGGACGAGAA
CACCCTGCAGCACGCCTACCCCATCGACCACGAGGGCGCCGAGCCTGCCC
CCCAGGAACAGAACCTGTTCAGCAGCATCGAGATCGTGGAGCGGAGCAAC
TACATGGGCAACCCCTGGACCGAGTATATGGCCAAGTATGACATCGAGGAA
GTGCACGGCAGCGGCATCCGGGTGGACCTGGGCGAGGACGCCGAGGTGG
CCGGCACCCAGTATCGGCTGCCCAGCGGCAAGTGCCCCGTGTTCGGCAAG
GGCATCATCATCGAGAACAGCAAGACCACCTTCCTGACCCCCGTGGCCACC
GGCAATCAGTATCTGAAGGACGGCGGCTTCGCCTTCCCCCCCACCGAGCC
CCTGATGAGCCCCATGACCCTGGACGAGATGCGGCACTTCTACAAGGACAA
CAAGTATGTGAAGAACCTGGACGAGCTGACCCTGTGCAGCCGGCACGCCG
GCAACATGATCCCCGACAACGACAAGAACAGCAACTACAAGTATCCCGCCG
TGTATGACGACAAGGATAAGAAGTGCCACATCCTGTATATCGCCGCCCAGG
AAAACAACGGCCCCAGATACTGCAACAAGGACGAGAGCAAGCGGAACAGC
ATGTTCTGCTTCAGACCCGCCAAGGACATCAGCTTCCAGAACCTAGTCTACC
TGAGCAAGAACGTGGTGGACAACTGGGAGAAAGTGTGCCCCCGGAAGAAC
CTGCAGAACGCCAAGTTCGGCCTGTGGGTGGACGGCAACTGCGAGGACAT
CCCCCACGTGAACGAGTTCCCCGCCATCGACCTGTTCGAGTGCAACAAGCT
GGTGTTCGAGCTGTCCGCCAGCGACCAGCCCAAGCAGTATGAGCAGCACC
TGACCGACTACGAGAAGATCAAAGAGGGCTTCAAGAACAAGAACCGCGAGA
TGATCAAGAGCGCCTTCCTGCCCACCGGCGCCTTCAAGGCCGACAGATACA
AGAGCCACGGCAAGGGCTACAACTGGGGCAACTACAACACCGAGACCCAG
AAGTGCGAGATCTTCAACGTGAAGCCCACCTGCCTGATCAATGACAAGAAC
TACATCGCCACCACCGCCCTGAGCCACCCCATCGAGGTGGAGAACAACTTC
CCCTGCAGCCTGTATAAGGACGAGATCATGAAAGAGATCGAGCGGGAGAG
CAAGAGGATCAAGCTGAACGACAACGACGACGAGGGCAACAAGAAGATCAT
CGCCCCCAGGATCTTCATCAGCGACGATAAGGACAGCCTGAAGTGCCCCTG
CGACCCCGAGATGGTGTCCCAAAGTACATGCCGGTTCTTCGTGTGCAAGTG
CGTGGAGAGAAGGGCCGAGGTGACCAGCAACAACGAGGTGGTGGTGAAAG
AGGAATACAAGGACGAATACGCCGACATCCCCGAGCACAAGCCCACCTACG
ACAAGATGAAGTGA
TPA-PyMSP1₄₂-PK - gene with tpa leader and PK tag.
SEQ ID NO: 2
atggatgcaatgaagagagggctctgctgtgtgctgctgctgtgtggagcagtcttcgtttcgcccagccaggaa
atccatgcccgattcagaagaCTCGActccgaagatgcaccagaaaaagatattctttccgaatttacaaatg
aaagtttgtatgtatacacaaaaaggttgggtagtacatataaatcattaaagaaacacatgttaagagaattttc
aacaattaaagaagacatgacaaatggattaaataataaatcacaaaaaagaaatgatttccttgaagtattaa
gccatgaattagatttattcaaagatttaagtaccaacaaatatgttattagaaatccatatcaattattagataatga
taaaaaagacaaacaaatagtaaacttaaaatatgctactaaaggtataaatgaagatatagaaacaactact
gacggaattaaattctttaacaaaatggttgaattatacaacactcaattagctgcagtaaaggaacaaattgcta
ccatagaagctgaaactaacgataccaataaagaagaaaaaaagaaatatattccaatccttgaagatcttaa
aggattatatgaaaccgtaataggtcaagcagaagaatattcagaagaattacaaaatagacttgataattata
aaaatgaaaaagctgaatttgaaatattaacaaaaaatttagaaaaatacatacaaattgacgaaaaacttga
cgaatttgtagaacatgcagaaaataataaacacatagcctcaatagctttaaacaacttaaataaatctggttta
gtaggagaaggtgaatcaaagaaaatattagcaaaaatgcttaacatggatggtatggatttattaggtgtagac
cctaaacatgtatgtgttgatacaagagatattcctaaaaatgctggatgttttagagatgataatggtactgaaga
atggagatgtttattaggttacaaaaaaggtgaaggtaatacatgtgtagaaaataataatcctacttgtgatatca
acaatggtggatgtgatccaactgctagttgtcaaaatgcggaaagtacggaaaattccaaaaaaattatatgta
catgtaaagaaccaacccctaatgcatattatgaaggtgtattctgtagttcttccagctttatgggaattcctaaccc
tttgctaggtctagactga
PfMSP1₁₅.
SEQ ID NO: 3
ATGAAGATCATCTTCTTCCTGTGCTCTTTCCTGTTCTTCATCATCAACACCCA
GTGCGTGACCCACGAGAGCTACCAGGAGCTGGTGAAGAAGCTGGAGGCCC
TGGAGGACGCCGTGCTGACCGGCTACAGCCTGTTCCAGAAAGAGAAGATG
GTGCTGAACGAGCTGTTCGACCTGACCAACCACATGCTGACCCTGTGCGAC
AACATCCACGGCTTCAAGTACCTGATCGACGGCTACGAGGAGATCAACGAG
CTGCTGTACAAGCTGAACTTCTACTTCGACCTGCTGCGCGCCAAGCTGAAC
GACGTGTGCGCCAACGACTACTGCCAGATCCCCTTCAACCTGAAGATCCGC
GCCAACGAGCTGGACGTGCTGAAGAAACTGGTGTTCGGCTACCGGAAGCC
CCTGGACAACATCAAGGACAACGTGGGCAAGATGGAGGACTACATCAAGAA
GAACAAGACCACCATCGCCAACATTAACGAGCTGATCGAGGGCAGCAAGAA
AACCATCGACCAGAACAAGAACGCCGACAACGAGGAGGGCAAGAAGAAGC
TGTACCAGGCCCAGTACGACCTGAGCATCTACAACAAGCAGCTGGAGGAG
GCCCACAACCTGATCAGCGTGCTGGAGAAGCGGATCGACACCCTGAAGAA
GAACGAGAACATCAAGATCAAGGAGATCGCCAAGACCATCAAGTTCAACAT
CGACTCCCTGTTCACCGACCCCCTGGAGCTGGAGTACTACCTGCGCGAGAA
GAATAAGAAGATGCAGATCAAGAAGCTGACCCTGCTGAAGGAGCAGCTGGA
AAGCAAGCTGAACAGCCTGAACAACCCCCACAACGTGCTGCAGAACTTCAG
CGTGTTCTTCAACAAGAAGAAGGAGGCCGAGATCGCCGAAACCGAGAACAC
CCTGGAGAATACCAAGATCCTGCTGAAGCACTACAAGGGCCTGGTGAAGTA
CTACAACGGCGAGAGCAGCCCCCTGAAAACCCTGAGCGAAGTGAGCATCC
AGACCGAGGACAACTACGCCAACCTGGAGGGCCAAGTGGTCACCGGCGAG
GCCGTGACCACAAGCGTGATCGACAATATCCTGAGCAAGATCGAGAACGAG
TACGAAGTGCTGTACCTGAAGCCTCTGGCCGGCGTGTACCGGAGCCTGAA
GAAACAGCTGGAGAACAACGTGATGACCTTCAACGTGAACGTGAAGGACAT
CCTGAACAGCCGGTTCAACAAGCGCGAGAACTTCAAGAACGTGCTGGAGTC
CGACCTGATCCCCTACAAGGACCTGACCAGCAGCAACTACGTGGTGAAGGA
CCCCTACAAGTTCCTGAACAAGGAGAAGCGCGACAAGTTTCTGTCCAGCTA
CAACTACATTAAGGACAGCATCGACACCGACATCAACTTCGCCAACGACGT
GCTGGGCTACTACAAGATCCTGAGCGAGAAGTACAAGAGCGACCTGGATAG
CATCAAGAAGTACATCAACGACAAGCAGGGCGAGAACGAGAAGTACCTGCC
CTTCCTGAATAACATCGAGACCCTGTACAAGACCGTGAACGACAAGATCGA
CCTGTTCGTGATCCACCTGGAGGCCAAAGTGCTGAACTACACCTACGAGAA
GAGCAACGTGGAAGTGAAGATTAAGGAGCTGAACTACCTGAAAACCATCCA
GGACAAGCTGGCCGACTTCAAGAAGAATAACAACTTCGTGGGCATCGCCGA
TCTGAGCACCGACTACAACCACAACAACCTGCTGACCAAGTTCCTGTCCAC
CGGCATGGTGTTCGAGAACCTGCTGAAGAGCGTGCTGAGCAACCTGCTGG
ACTGGAAGCTGGCCCGCTACGTGAAGCACTTCACCACCCCCATGCGGAAAA
AGACCATGATCCAGCAGAGCGGAGGGGGACCCGGGGGAGGGGACCAAGT
CGTGACCGGCGAAGCCATCAGCGTGACCATGGATAACATCCTGAGCGGCTT
CGAAAACGAATACGACGTGATCTATCTGAAACCCCTGGCCGGCGTGTATCG
GTCTCTGAAGAAGCAGATCGAGAAGAACATCTTCACCTTCAATCTGAACCTG
AACGATATCCTGAATAGCCGCCTGAAGAAGCGCAAGTACTTCCTGGACGTG
CTGGAGAGCGACCTGATGCAGTTCAAGCACATCAGCAGCAACGAGTACATC
ATCGAGGACAGCTTCAAGCTGCTGAACAGCGAGCAGAAGAACACACTGCTG
AAGTCTTACAAGTATATCAAGGAGAGCGTGGAGAACGATATCAAGTTCGCC
CAGGAGGGCATCAGCTACTACGAGAAAGTGCTGGCCAAGTACAAGGACGAT
CTGGAGTCCATCAAGAAAGTGATCAAGGAGGAGAAGGAGAAGTTCCCCAGC
AGCCCCCCCACCACCCCCCCCAGCCCCGCCAAGACCGACGAGCAGAAGAA
GGAGAGCAAGTTCCTGCCTTTTCTGACCAATATCGAGACACTGTATAACAAC
CTGGTGAATAAGATCGACGACTACCTGATCAATCTGAAGGCCAAGATCAAC
GATTGCAACGTGGAGAAGGACGAGGCCCACGTGAAGATCACCAAGCTGAG
CGATCTGAAAGCCATCGACGATAAGATCGATCTGTTCAAGAACCCCTACGA
CTTCGAGGCCATTAAGAAGCTGATCAACGACGACACCAAGAAGGACATGCT
GGGCAAGCTGCTGTCTACCGGCCTGGTGCAGAATTTCCCCAACACCATCAT
CAGCAAGCTGATCGAAGGGAAGTTCCAGGATATGCTGAACATCGCCCAGCA
CCAGTGCGTGAAGAAGCAGATCCCCGAGAACAGCGGCTGCTTCCGGCACC
TGGACGAGCGCGAGGAGTGGAAGTGCCTGCTGAATTACAAGCAGGAGGGC
GACAAGTGCGTGGAGAATCCCAACCCCACCTGCAACGAGAACAACGGCGG
CTGCGACGCCGACGCCACCTGCACCGAGGAGGACAGCGGCAGCAGCCGG
AAGAAGATCACCTGCGAGTGCACCAAGCCCGACAGCTACCCCCTGTTCGAC
GGCATCTTCTGCAGCAGCTCCAACTTAATATTATACAGCTTCATCAAGTACAT
CCCCATCCTGGAGGACCTGTGA
GFP.
SEQ ID NO: 4
ATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGG
GCGAGGGCGATGCCACCTACGGCAAGCTGACCCTGAAGTTCATCTGCACC
ACCGGCAAGCTGCCCGTGCCCTGGCCCACCCTCGTGACCACCCTGACCTA
CGGCGTGCAGTGCTTCAGCCGCTACCCCGACCACATGAAGCAGCACGACT
TCTTCAAGTCCGCCATGCCCGAAGGCTACGTCCAGGAGCGCACCATCTTCT
TCAAGGACGACGGCAACTACAAGACCCGCGCCGAGGTGAAGTTCGAGGGC
GACACCCTGGTGAACCGCATCGAGCTGAAGGGCATCGACTTCAAGGAGGA
CGGCAACATCCTGGGGCACAAGCTGGAGTACAACTACAACAGCCACAACGT
CTATATCATGGCCGACAAGCAGAAGAACGGCATCAAGGTGAACTTCAAGAT
CCGCCACAACATCGAGGACGGCAGCGTGCAGCTCGCCGACCACTACCAGC
AGAACACCCCCATCGGCGACGGCCCCGTGCTGCTGCCCGACAACCACTAC
CTGAGCACCCAGTCCGCCCTGAGCAAAGACCCCAACGAGAAGCGCGATCA
CATGGTCCTGCTGGAGTTCGTGACCGCCGCCGGGATCACTCTCGGCATGG
ACGAGCTGTACAAGTAA
ME-TRAP.
SEQ ID NO: 5
atgggtatgatcaacgcctacttggacaagttgatctccaagtacgaagacgaaatctcctacatcccatctgcc
gaaaagatcggatctaagccgaacgacaagtccttgtataaacctaaggacgaattggactacaagccaatc
gttcaatacgacaacttcggatctgcctccaagaacaaggaaaaggctttgatcatcggtatcgctggtggtttgg
ccttgttgatgaaccctaatgacccaaacagaaacgtcagatctcacttgggtaacgttaagtacttggttaagtctt
tgtacgatgaacacatcttattgatggactgttctggttctattggatctgacccaaacgctaacccaaacgttgacc
caaacgccaacccaaacgtccaagttcacttccaaccattgcctccggccgttgtcaagttgcaattcatcaagg
ccaactctaagttcatcggtatcaccgaaggatcttacttgaacaaaattcaaaactctttgatggaaaagttgaa
agaattggaaaaggctacttctgtcttggctggtttgggatctaacgctaatccaaacgcaaatccgaacgccaat
cctaacgcgaatcccgacgaatggtctccatgttctgtcacttgtggtaagggtactcgctctagaaagagagaa
ggatccaaaataatgaatcatcttgggaatgttaaatatttagtcattgtgtttttgattttctttgatttgtttctagttaatg
gtagagatgtgcaaaacaatatagtggatgaaataaaatatagtgaagaagtatgtaatgatcaggtagatcttt
accttctaatggattgttctggaagtatacgtcgtcataattgggtgaaccatgcagtacctctagctatgaaattgat
acaacaattaaatcttaatgataatgcaattcacttatatgttaatgttttttcaaacaatgcaaaagaaattattagat
tacatagtgatgcatctaaaaacaaagagaaggctttaattattataaggtcactcttaagtacaaatcttccatat
ggtagaacaaacttaactgatgcactgttacaagtaagaaaacatttaaatgaccgaatcaatagagagaatg
ctaatcaattagttgttatattaacagatggaattccagatagtattcaagattcattaaaagaatcaagaaaattaa
gtgatcgtggtgttaaaatagctgtttttggtattggacaaggtattaatgtagctttcaacagatttcttgtaggttgtca
tccatcagatggtaaatgtaacttgtatgctgattctgcatgggaaaatgtaaaaaatgttatcggaccctttatgaa
ggctgtttgtgttgaagtagaaaaaacagcaagttgtggtgtttgggacgaatggtctccatgtagtgtaacttgtgg
taaaggtaccaggtcaagaaaaagagaaatcttacacgaaggatgtacaagtgaaatacaagaacaatgtg
aagaagaaagatgtcctccaaaatgggaaccattagatgttccagatgaacccgaagatgatcaacctagac
caagaggagataattcttctgtccaaaaaccagaagaaaatataatagataataatccacaagaaccttcacc
aaatccagaagaaggaaaggatgaaaatccaaacggatttgatttagatgaaaatccagaaaatccaccaa
atccagatattcctgaacaaaaaccaaatatacctgaagattcagaaaaagaagtaccttctgatgttccaaaa
aatccagaagacgatcgagaagaaaactttgatattccaaagaaacccgaaaataagcacgataatcaaaat
aatttaccaaatgataaaagtgatagaaatattccatattcaccattacctccaaaagttttggataatgaaagga
aacaaagtgacccccaaagtcaagataataatggaaataggcacgtacctaatagtgaagatagagaaaca
cgtccacatggtagaaataatgaaaatagatcatacaatagaaaatataacgatactccaaaacatcctgaaa
gggaagaacatgaaaagccagataataataaaaaaaaaggagaatcagataataaatataaaattgcaggt
ggaatagctggaggattagctttactcgcatgtgctggacttgcttataaattcgtagtaccaggagcagcaacac
cctatgccggagaacctgcaccttttgatgaaacattaggtgaagaagataaagatttggacgaacctgaacaa
ttcagattacctgaagaaaacgagtggaattaa
PfM128.
SEQ ID NO: 6
ATGAAGATCATCTTCTTCCTGTGCTCTTTCCTGTTCTTCATCATCAACACCCA
GTGCGTGACCCACGAGAGCTACCAGGAGCTGGTGAAGAAGCTGGAGGCCC
TGGAGGACGCCGTGCTGACCGGCTACAGCCTGTTCCAGAAAGAGAAGATG
GTGCTGAACGAGCTGTTCGACCTGACCAACCACATGCTGACCCTGTGCGAC
AACATCCACGGCTTCAAGTACCTGATCGACGGCTACGAGGAGATCAACGAG
CTGCTGTACAAGCTGAACTTCTACTTCGACCTGCTGCGCGCCAAGCTGAAC
GACGTGTGCGCCAACGACTACTGCCAGATCCCCTTCAACCTGAAGATCCGC
GCCAACGAGCTGGACGTGCTGAAGAAACTGGTGTTCGGCTACCGGAAGCC
CCTGGACAACATCAAGGACAACGTGGGCAAGATGGAGGACTACATCAAGAA
GAACAAGACCACCATCGCCAACATTAACGAGCTGATCGAGGGCAGCAAGAA
AACCATCGACCAGAACAAGAACGCCGACAACGAGGAGGGCAAGAAGAAGC
TGTACCAGGCCCAGTACGACCTGAGCATCTACAACAAGCAGCTGGAGGAG
GCCCACAACCTGATCAGCGTGCTGGAGAAGCGGATCGACACCCTGAAGAA
GAACGAGAACATCAAGATCAAGGAGATCGCCAAGACCATCAAGTTCAACAT
CGACTCCCTGTTCACCGACCCCCTGGAGCTGGAGTACTACCTGCGCGAGAA
GAATAAGAAGATGCAGATCAAGAAGCTGACCCTGCTGAAGGAGCAGCTGGA
AAGCAAGCTGAACAGCCTGAACAACCCCCACAACGTGCTGCAGAACTTCAG
CGTGTTCTTCAACAAGAAGAAGGAGGCCGAGATCGCCGAAACCGAGAACAC
CCTGGAGAATACCAAGATCCTGCTGAAGCACTACAAGGGCCTGGTGAAGTA
CTACAACGGCGAGAGCAGCCCCCTGAAAACCCTGAGCGAAGTGAGCATCC
AGACCGAGGACAACTACGCCAACCTGGAGGGCCAAGTGGTCACCGGCGAG
GCCGTGACCCCCAGCGTGATCGACAACATCCTGAGCAAGATCGAGAACGA
GTACGAGGTGCTGTACCTGAAGCCCCTGGCCGGCGTGTACAGAAGCCTGA
AGAAGCAGCTGGAAAACAACGTGATGACCTTCAACGTGAACGTGAAGGACA
TCCTGAACAGCCGGTTCAACAAGCGGGAGAACTTCAAGAACGTGCTGGAAA
GCGACCTGATCCCCTACAAGGACCTGACCAGCAGCAACTACGTGGTGAAG
GACCCCTACAAGTTCCTGAACAAAGAGAAGCGGGATAAGTTCCTGAGCAGC
TACAACTACATCAAGGACAGCATCGACACCGACATCAACTTCGCCAACGAC
GTGCTGGGCTACTACAAGATCCTGAGCGAGAAGTACAAGAGCGACCTGGAC
AGCATCAAGAAGTACATCAACGACAAGCAGGGCGAGAACGAGAAGTACCTG
CCCTTCCTGAATAACATCGAGACCCTGTACAAGACCGTGAACGACAAGATC
GACCTGTTCGTGATCCACCTGGAAGCCAAGGTGCTGAACTACACCTACGAG
AAGAGCAACGTGGAGGTGAAGATCAAAGAGCTGAACTACCTGAAAACCATC
CAGGACAAGCTGGCCGACTTCAAGAAGAACAACAACTTCGTCGGCATCGCC
GACCTGAGCACCGACTACAACCACAACAACCTGCTGACCAAGTTCCTGTCC
ACCGGCATGGTGTTCGAGAACCTGGCCAAGACAGTGCTGTCCAACCTGCTG
GACGGCAACCTGCAGGGCATGCTCAATATCGCACAGCATCAGTGTGTCAAA
AAACAGATTCCTCAGAACTCCGGCTGCTTTAGACACCTGGATGAACGGGAA
GAATGGAAGTGTCTGCTCAACTATAAACAGGAAGGTGATAAGTGTGTCGAG
AACCCTAACCCTACCTGTAATGAGAATAATGGGGGCTGTGATGCCGATGCC
AAATGTACCGAAGAAGATTCCGGCTCCAATGGCAAGAAAATCACATGTGAAT
GTACCAAACCCGACTCCTACCCTCTCTTCGATGGGATCTTTTGCAGCTCCAG
TAATGGCGGCGGACCCGGGGGAGGGGACCAAGTCGTGACCGGCGAAGCC
ATCAGCGTGACCATGGATAACATCCTGAGCGGCTTCGAAAACGAATACGAC
GTGATCTATCTGAAACCCCTGGCCGGCGTGTATCGGTCTCTGAAGAAGCAG
ATCGAGAAGAACATCTTCACCTTCAATCTGAACCTGAACGATATCCTGAATA
GCCGCCTGAAGAAGCGCAAGTACTTCCTGGACGTGCTGGAGAGCGACCTG
ATGCAGTTCAAGCACATCAGCAGCAACGAGTACATCATCGAGGACAGCTTC
AAGCTGCTGAACAGCGAGCAGAAGAACACACTGCTGAAGTCTTACAAGTAT
ATCAAGGAGAGCGTGGAGAACGATATCAAGTTCGCCCAGGAGGGCATCAG
CTACTACGAGAAAGTGCTGGCCAAGTACAAGGACGATCTGGAGTCCATCAA
GAAAGTGATCAAGGAGGAGAAGGAGAAGTTCCCCAGCAGCCCCCCCACCA
CCCCCCCCAGCCCCGCCAAGACCGACGAGCAGAAGAAGGAGAGCAAGTTC
CTGCCTTTTCTGACCAATATCGAGACACTGTATAACAACCTGGTGAATAAGA
TCGACGACTACCTGATCAATCTGAAGGCCAAGATCAACGATTGCAACGTGG
AGAAGGACGAGGCCCACGTGAAGATCACCAAGCTGAGCGATCTGAAAGCC
ATCGACGATAAGATCGATCTGTTCAAGAACCCCTACGACTTCGAGGCCATTA
AGAAGCTGATCAACGACGACACCAAGAAGGACATGCTGGGCAAGCTGCTGT
CTACCGGCCTGGTGCAGAATTTCCCCAACACCATCATCAGCAAGCTGATCG
AAGGGAAGTTCCAGGATATGCTGAACATCGCCCAGCACCAGTGCGTGAAGA
AGCAGATCCCCGAGAACAGCGGCTGCTTCCGGCACCTGGACGAGCGCGAG
GAGTGGAAGTGCCTGCTGAATTACAAGCAGGAGGGCGACAAGTGCGTGGA
GAATCCCAACCCCACCTGCAACGAGAACAACGGCGGCTGCGACGCCGACG
CCACCTGCACCGAGGAGGACAGCGGCAGCAGCCGGAAGAAGATCACCTGC
GAGTGCACCAAGCCCGACAGCTACCCCCTGTTCGACGGCATCTTCTGCAGC
AGCTCCAACTTAATATTATATTCCTTTATCTGA

AdHu5 genome.

SEQ ID NO: 7
Underlined region may be replaced with promoter + antigen + polyA sequence
TAACATCATCAATAATATACCTTATTTTGGATTGAAGCCAATATGATAATGAG
GGGGTGGAGTTTGTGACGTGGCGCGGGGCGTGGGAACGGGGCGGGTGAC
GTAGTAGTGTGGCGGAAGTGTGATGTTGCAAGTGTGGCGGAACACATGTAA
GCGACGGATGTGGCAAAAGTGACGTTTTTGGTGTGCGCCGGTGTACACAG
GAAGTGACAATTTTCGCGCGGTTTTAGGCGGATGTTGTAGTAAATTTGGGC
GTAACCGAGTAAGATTTGGCCATTTTCGCGGGAAAACTGAATAAGAGGAAG
TGAAATCTGAATAATTTTGTGTTACTCATAGCGCGTAATATTTGTCTAGGGCC
GCGGGGACTTTGACCGTTTACGTGGAGACTCGCCCAGGTGTTTTTCTCAGG
TGTTTTCCGCGTTCCGGGTCAAAGTTGGCGTTTTATTATTATAGTCAGTCGA
AGCTTGGATCCGGTACCTCTAGAATTCTCGAGCGGCCGCTAGCGACATCGA
TCACAAGTTTGTACAAAAAAGCTGAACGAGAAACGTAAAATGATATAAATATC
AATATATTAAATTAGATTTTGCATAAAAAACAGACTACATAATACTGTAAAACA
CAACATATCCAGTCACTATGGCGGCCGCATTAGGCACCCCAGGCTTTACAC
TTTATGCTTCCGGCTCGTATAATGTGTGGATTTTGAGTTAGGATCCGGCGAG
ATTTTCAGGAGCTAAGGAAGCTAAAATGGAGAAAAAAATCACTGGATATACC
ACCGTTGATATATCCCAATGGCATCGTAAAGAACATTTTGAGGCATTTCAGT
CAGTTGCTCAATGTACCTATAACCAGACCGTTCAGCTGGATATTACGGCCTT
TTTAAAGACCGTAAAGAAAAATAAGCACAAGTTTTATCCGGCCTTTATTCACA
TTCTTGCCCGCCTGATGAATGCTCATCCGGAATTCCGTATGGCAATGAAAGA
CGGTGAGCTGGTGATATGGGATAGTGTTCACCCTTGTTACACCGTTTTCCAT
GAGCAAACTGAAACGTTTTCATCGCTCTGGAGTGAATACCACGACGATTTCC
GGCAGTTTCTACACATATATTCGCAAGATGTGGCGTGTTACGGTGAAAACCT
GGCCTATTTCCCTAAAGGGTTTATTGAGAATATGTTTTTCGTCTCAGCCAATC
CCTGGGTGAGTTTCACCAGTTTTGATTTAAACGTGGCCAATATGGACAACTT
CTTCGCCCCCGTTTTCACCATGGGCAAATATTATACGCAAGGCGACAAGGT
GCTGATGCCGCTGGCGATTCAGGTTCATCATGCCGTCTGTGATGGCTTCCA
TGTCGGCAGAATGCTTAATGAATTACAACAGTACTGCGATGAGTGGCAGGG
CGGGGCGTAAACGCGTGGATCCGGCTTACTAAAAGCCAGATAACAGTATGC
GTATTTGCGCGCTGATTTTTGCGGTATAAGAATATATACTGATATGTATACCC
GAAGTATGTCAAAAAGAGGTGTGCTATGAAGCAGCGTATTACAGTGACAGTT
GACAGCGACAGCTATCAGTTGCTCAAGGCATATATGATGTCAATATCTCCGG
TCTGGTAAGCACAACCATGCAGAATGAAGCCCGTCGTCTGCGTGCCGAACG
CTGGAAAGCGGAAAATCAGGAAGGGATGGCTGAGGTCGCCCGGTTTATTGA
AATGAACGGCTCTTTTGCTGACGAGAACAGGGACTGGTGAAATGCAGTTTA
AGGTTTACACCTATAAAAGAGAGAGCCGTTATCGTCTGTTTGTGGATGTACA
GAGTGATATTATTGACACGCCCGGGCGACGGATGGTGATCCCCCTGGCCA
GTGCACGTCTGCTGTCAGATAAAGTCTCCCGTGAACTTTACCCGGTGGTGC
ATATCGGGGATGAAAGCTGGCGCATGATGACCACCGATATGGCCAGTGTGC
CGGTCTCCGTTATCGGGGAAGAAGTGGCTGATCTCAGCCACCGCGAAAATG
ACATCAAAAACGCCATTAACCTGATGTTCTGGGGAATATAAATGTCAGGCTC
CGTTATACACAGCCAGTCTGCAGGTCGACCATAGTGACTGGATATGTTGTGT
TTTACAGTATTATGTAGTCTGTTTTTTATGCAAAATCTAATTTAATATATTGAT
ATTTATATCATTTTACGTTTCTCGTTCAGCTTTCTTGTACAAAGTGGTGATCG
ATTCGACAGATCACTGAAATGTGTGGGCGTGGCTTAAGGGTGGGAAAGAAT
ATATAAGGTGGGGGTCTTATGTAGTTTTGTATCTGTTTTGCAGCAGCCGCCG
CCGCCATGAGCACCAACTCGTTTGATGGAAGCATTGTGAGCTCATATTTGAC
AACGCGCATGCCCCCATGGGCCGGGGTGCGTCAGAATGTGATGGGCTCCA
GCATTGATGGTCGCCCCGTCCTGCCCGCAAACTCTACTACCTTGACCTACG
AGACCGTGTCTGGAACGCCGTTGGAGACTGCAGCCTCCGCCGCCGCTTCA
GCCGCTGCAGCCACCGCCCGCGGGATTGTGACTGACTTTGCTTTCCTGAGC
CCGCTTGCAAGCAGTGCAGCTTCCCGTTCATCCGCCCGCGATGACAAGTTG
ACGGCTCTTTTGGCACAATTGGATTCTTTGACCCGGGAACTTAATGTCGTTT
CTCAGCAGCTGTTGGATCTGCGCCAGCAGGTTTCTGCCCTGAAGGCTTCCT
CCCCTCCCAATGCGGTTTAAAACATAAATAAAAAACCAGACTCTGTTTGGAT
TTGGATCAAGCAAGTGTCTTGCTGTCTTTATTTAGGGGTTTTGCGCGCGCGG
TAGGCCCGGGACCAGCGGTCTCGGTCGTTGAGGGTCCTGTGTATTTTTTCC
AGGACGTGGTAAAGGTGACTCTGGATGTTCAGATACATGGGCATAAGCCCG
TCTCTGGGGTGGAGGTAGCACCACTGCAGAGCTTCATGCTGCGGGGTGGT
GTTGTAGATGATCCAGTCGTAGCAGGAGCGCTGGGCGTGGTGCCTAAAAAT
GTCTTTCAGTAGCAAGCTGATTGCCAGGGGCAGGCCCTTGGTGTAAGTGTT
TACAAAGCGGTTAAGCTGGGATGGGTGCATACGTGGGGATATGAGATGCAT
CTTGGACTGTATTTTTAGGTTGGCTATGTTCCCAGCCATATCCCTCCGGGGA
TTCATGTTGTGCAGAACCACCAGCACAGTGTATCCGGTGCACTTGGGAAATT
TGTCATGTAGCTTAGAAGGAAATGCGTGGAAGAACTTGGAGACGCCCTTGT
GACCTCCAAGATTTTCCATGCATTCGTCCATAATGATGGCAATGGGCCCACG
GGCGGCGGCCTGGGCGAAGATATTTCTGGGATCACTAACGTCATAGTTGTG
TTCCAGGATGAGATCGTCATAGGCCATTTTTACAAAGCGCGGGCGGAGGGT
GCCAGACTGCGGTATAATGGTTCCATCCGGCCCAGGGGCGTAGTTACCCTC
ACAGATTTGCATTTCCCACGCTTTGAGTTCAGATGGGGGGATCATGTCTACC
TGCGGGGCGATGAAGAAAACGGTTTCCGGGGTAGGGGAGATCAGCTGGGA
AGAAAGCAGGTTCCTGAGCAGCTGCGACTTACCGCAGCCGGTGGGCCCGT
AAATCACACCTATTACCGGGTGCAACTGGTAGTTAAGAGAGCTGCAGCTGC
CGTCATCCCTGAGCAGGGGGGCCACTTCGTTAAGCATGTCCCTGACTCGCA
TGTTTTCCCTGACCAAATCCGCCAGAAGGCGCTCGCCGCCCAGCGATAGCA
GTTCTTGCAAGGAAGCAAAGTTTTTCAACGGTTTGAGACCGTCCGCCGTAG
GCATGCTTTTGAGCGTTTGACCAAGCAGTTCCAGGCGGTCCCACAGCTCGG
TCACCTGCTCTACGGCATCTCGATCCAGCATATCTCCTCGTTTCGCGGGTTG
GGGCGGCTTTCGCTGTACGGCAGTAGTCGGTGCTCGTCCAGACGGGCCAG
GGTCATGTCTTTCCACGGGCGCAGGGTCCTCGTCAGCGTAGTCTGGGTCAC
GGTGAAGGGGTGCGCTCCGGGCTGCGCGCTGGCCAGGGTGCGCTTGAGG
CTGGTCCTGCTGGTGCTGAAGCGCTGCCGGTCTTCGCCCTGCGCGTCGGC
CAGGTAGCATTTGACCATGGTGTCATAGTCCAGCCCCTCCGCGGCGTGGCC
CTTGGCGCGCAGCTTGCCCTTGGAGGAGGCGCCGCACGAGGGGCAGTGC
AGACTTTTGAGGGCGTAGAGCTTGGGCGCGAGAAATACCGATTCCGGGGA
GTAGGCATCCGCGCCGCAGGCCCCGCAGACGGTCTCGCATTCCACGAGCC
AGGTGAGCTCTGGCCGTTCGGGGTCAAAAACCAGGTTTCCCCCATGCTTTT
TGATGCGTTTCTTACCTCTGGTTTCCATGAGCCGGTGTCCACGCTCGGTGA
CGAAAAGGCTGTCCGTGTCCCCGTATACAGACTTGAGAGGCCTGTCCTCGA
GCGGTGTTCCGCGGTCCTCCTCGTATAGAAACTCGGACCACTCTGAGACAA
AGGCTCGCGTCCAGGCCAGCACGAAGGAGGCTAAGTGGGAGGGGTAGCG
GTCGTTGTCCACTAGGGGGTCCACTCGCTCCAGGGTGTGAAGACACATGTC
GCCCTCTTCGGCATCAAGGAAGGTGATTGGTTTGTAGGTGTAGGCCACGTG
ACCGGGTGTTCCTGAAGGGGGGCTATAAAAGGGGGTGGGGGCGCGTTCGT
CCTCACTCTCTTCCGCATCGCTGTCTGCGAGGGCCAGCTGTTGGGGTGAGT
ACTCCCTCTGAAAAGCGGGCATGACTTCTGCGCTAAGATTGTCAGTTTCCAA
AAACGAGGAGGATTTGATATTCACCTGGCCCGCGGTGATGCCTTTGAGGGT
GGCCGCATCCATCTGGTCAGAAAAGACAATCTTTTTGTTGTCAAGCTTGGTG
GCAAACGACCCGTAGAGGGCGTTGGACAGCAACTTGGCGATGGAGCGCAG
GGTTTGGTTTTTGTCGCGATCGGCGCGCTCCTTGGCCGCGATGTTTAGCTG
CACGTATTCGCGCGCAACGCACCGCCATTCGGGAAAGACGGTGGTGCGCT
CGTCGGGCACCAGGTGCACGCGCCAACCGCGGTTGTGCAGGGTGACAAG
GTCAACGCTGGTGGCTACCTCTCCGCGTAGGCGCTCGTTGGTCCAGCAGA
GGCGGCCGCCCTTGCGCGAGCAGAATGGCGGTAGGGGGTCTAGCTGCGT
CTCGTCCGGGGGGTCTGCGTCCACGGTAAAGACCCCGGGCAGCAGGCGC
GCGTCGAAGTAGTCTATCTTGCATCCTTGCAAGTCTAGCGCCTGCTGCCAT
GCGCGGGCGGCAAGCGCGCGCTCGTATGGGTTGAGTGGGGGACCCCATG
GCATGGGGTGGGTGAGCGCGGAGGCGTACATGCCGCAAATGTCGTAAACG
TAGAGGGGCTCTCTGAGTATTCCAAGATATGTAGGGTAGCATCTTCCACCG
CGGATGCTGGCGCGCACGTAATCGTATAGTTCGTGCGAGGGAGCGAGGAG
GTCGGGACCGAGGTTGCTACGGGCGGGCTGCTCTGCTCGGAAGACTATCT
GCCTGAAGATGGCATGTGAGTTGGATGATATGGTTGGACGCTGGAAGACGT
TGAAGCTGGCGTCTGTGAGACCTACCGCGTCACGCACGAAGGAGGCGTAG
GAGTCGCGCAGCTTGTTGACCAGCTCGGCGGTGACCTGCACGTCTAGGGC
GCAGTAGTCCAGGGTTTCCTTGATGATGTCATACTTATCCTGTCCCTTTTTTT
TCCACAGCTCGCGGTTGAGGACAAACTCTTCGCGGTCTTTCCAGTACTCTT
GGATCGGAAACCCGTCGGCCTCCGAACGGTAAGAGCCTAGCATGTAGAACT
GGTTGACGGCCTGGTAGGCGCAGCATCCCTTTTCTACGGGTAGCGCGTATG
CCTGCGCGGCCTTCCGGAGCGAGGTGTGGGTGAGCGCAAAGGTGTCCCTG
ACCATGACTTTGAGGTACTGGTATTTGAAGTCAGTGTCGTCGCATCCGCCCT
GCTCCCAGAGCAAAAAGTCCGTGCGCTTTTTGGAACGCGGATTTGGCAGGG
CGAAGGTGACATCGTTGAAGAGTATCTTTCCCGCGCGAGGCATAAAGTTGC
GTGTGATGCGGAAGGGTCCCGGCACCTCGGAACGGTTGTTAATTACCTGG
GCGGCGAGCACGATCTCGTCAAAGCCGTTGATGTTGTGGCCCACAATGTAA
AGTTCCAAGAAGCGCGGGATGCCCTTGATGGAAGGCAATTTTTTAAGTTCCT
CGTAGGTGAGCTCTTCAGGGGAGCTGAGCCCGTGCTCTGAAAGGGCCCAG
TCTGCAAGATGAGGGTTGGAAGCGACGAATGAGCTCCACAGGTCACGGGC
CATTAGCATTTGCAGGTGGTCGCGAAAGGTCCTAAACTGGCGACCTATGGC
CATTTTTTCTGGGGTGATGCAGTAGAAGGTAAGCGGGTCTTGTTCCCAGCG
GTCCCATCCAAGGTTCGCGGCTAGGTCTCGCGCGGCAGTCACTAGAGGCT
CATCTCCGCCGAACTTCATGACCAGCATGAAGGGCACGAGCTGCTTCCCAA
AGGCCCCCATCCAAGTATAGGTCTCTACATCGTAGGTGACAAAGAGACGCT
CGGTGCGAGGATGCGAGCCGATCGGGAAGAACTGGATCTCCCGCCACCAA
TTGGAGGAGTGGCTATTGATGTGGTGAAAGTAGAAGTCCCTGCGACGGGCC
GAACACTCGTGCTGGCTTTTGTAAAAACGTGCGCAGTACTGGCAGCGGTGC
ACGGGCTGTACATCCTGCACGAGGTTGACCTGACGACCGCGCACAAGGAA
GCAGAGTGGGAATTTGAGCCCCTCGCCTGGCGGGTTTGGCTGGTGGTCTT
CTACTTCGGCTGCTTGTCCTTGACCGTCTGGCTGCTCGAGGGGAGTTACGG
TGGATCGGACCACCACGCCGCGCGAGCCCAAAGTCCAGATGTCCGCGCGC
GGCGGTCGGAGCTTGATGACAACATCGCGCAGATGGGAGCTGTCCATGGT
CTGGAGCTCCCGCGGCGTCAGGTCAGGCGGGAGCTCCTGCAGGTTTACCT
CGCATAGACGGGTCAGGGCGCGGGCTAGATCCAGGTGATACCTAATTTCCA
GGGGCTGGTTGGTGGCGGCGTCGATGGCTTGCAAGAGGCCGCATCCCCG
CGGCGCGACTACGGTACCGCGCGGCGGGCGGTGGGCCGCGGGGGTGTC
CTTGGATGATGCATCTAAAAGCGGTGACGCGGGCGAGCCCCCGGAGGTAG
GGGGGGCTCCGGACCCGCCGGGAGAGGGGGCAGGGGCACGTCGGCGCC
GCGCGCGGGCAGGAGCTGGTGCTGCGCGCGTAGGTTGCTGGCGAACGCG
ACGACGCGGCGGTTGATCTCCTGAATCTGGCGCCTCTGCGTGAAGACGAC
GGGCCCGGTGAGCTTGAGCCTGAAAGAGAGTTCGACAGAATCAATTTCGGT
GTCGTTGACGGCGGCCTGGCGCAAAATCTCCTGCACGTCTCCTGAGTTGTC
TTGATAGGCGATCTCGGCCATGAACTGCTCGATCTCTTCCTCCTGGAGATCT
CCGCGTCCGGCTCGCTCCACGGTGGCGGCGAGGTCGTTGGAAATGCGGG
CCATGAGCTGCGAGAAGGCGTTGAGGCCTCCCTCGTTCCAGACGCGGCTG
TAGACCACGCCCCCTTCGGCATCGCGGGCGCGCATGACCACCTGCGCGAG
ATTGAGCTCCACGTGCCGGGCGAAGACGGCGTAGTTTCGCAGGCGCTGAA
AGAGGTAGTTGAGGGTGGTGGCGGTGTGTTCTGCCACGAAGAAGTACATAA
CCCAGCGTCGCAACGTGGATTCGTTGATATCCCCCAAGGCCTCAAGGCGCT
CCATGGCCTCGTAGAAGTCCACGGCGAAGTTGAAAAACTGGGAGTTGCGC
GCCGACACGGTTAACTCCTCCTCCAGAAGACGGATGAGCTCGGCGACAGT
GTCGCGCACCTCGCGCTCAAAGGCTACAGGGGCCTCTTCTTCTTCTTCAAT
CTCCTCTTCCATAAGGGCCTCCCCTTCTTCTTCTTCTGGCGGCGGTGGGGG
AGGGGGGACACGGCGGCGACGACGGCGCACCGGGAGGCGGTCGACAAAG
CGCTCGATCATCTCCCCGCGGCGACGGCGCATGGTCTCGGTGACGGCGCG
GCCGTTCTCGCGGGGGCGCAGTTGGAAGACGCCGCCCGTCATGTCCCGGT
TATGGGTTGGCGGGGGGCTGCCATGCGGCAGGGATACGGCGCTAACGATG
CATCTCAACAATTGTTGTGTAGGTACTCCGCCGCCGAGGGACCTGAGCGAG
TCCGCATCGACCGGATCGGAAAACCTCTCGAGAAAGGCGTCTAACCAGTCA
CAGTCGCAAGGTAGGCTGAGCACCGTGGCGGGCGGCAGCGGGCGGCGGT
CGGGGTTGTTTCTGGCGGAGGTGCTGCTGATGATGTAATTAAAGTAGGCGG
TCTTGAGACGGCGGATGGTCGACAGAAGCACCATGTCCTTGGGTCCGGCC
TGCTGAATGCGCAGGCGGTCGGCCATGCCCCAGGCTTCGTTTTGACATCG
GCGCAGGTCTTTGTAGTAGTCTTGCATGAGCCTTTCTACCGGCACTTCTTCT
TCTCCTTCCTCTTGTCCTGCATCTCTTGCATCTATCGCTGCGGCGGCGGCG
GAGTTTGGCCGTAGGTGGCGCCCTCTTCCTCCCATGCGTGTGACCCCGAA
GCCCCTCATCGGCTGAAGCAGGGCTAGGTCGGCGACAACGCGCTCGGCTA
ATATGGCCTGCTGCACCTGCGTGAGGGTAGACTGGAAGTCATCCATGTCCA
CAAAGCGGTGGTATGCGCCCGTGTTGATGGTGTAAGTGCAGTTGGCCATAA
CGGACCAGTTAACGGTCTGGTGACCCGGCTGCGAGAGCTCGGTGTACCTG
AGACGCGAGTAAGCCCTCGAGTCAAATACGTAGTCGTTGCAAGTCCGCACC
AGGTACTGGTATCCCACCAAAAAGTGCGGCGGCGGCTGGCGGTAGAGGGG
CCAGCGTAGGGTGGCCGGGGCTCCGGGGGCGAGATCTTCCAACATAAGGC
GATGATATCCGTAGATGTACCTGGACATCCAGGTGATGCCGGCGGCGGTG
GTGGAGGCGCGCGGAAAGTCGCGGACGCGGTTCCAGATGTTGCGCAGCG
GCAAAAAGTGCTCCATGGTCGGGACGCTCTGGCCGGTCAGGCGCGCGCAA
TCGTTGACGCTCTAGACCGTGCAAAAGGAGAGCCTGTAAGCGGGCACTCTT
CCGTGGTCTGGTGGATAAATTCGCAAGGGTATCATGGCGGACGACCGGGG
TTCGAGCCCCGTATCCGGCCGTCCGCCGTGATCCATGCGGTTACCGCCCG
CGTGTCGAACCCAGGTGTGCGACGTCAGACAACGGGGGAGTGCTCCTTTT
GGCTTCCTTCCAGGCGCGGCGGCTGCTGCGCTAGCTTTTTTGGCCACTGG
CCGCGCGCAGCGTAAGCGGTTAGGCTGGAAAGCGAAAGCATTAAGTGGCT
CGCTCCCTGTAGCCGGAGGGTTATTTTCCAAGGGTTGAGTCGCGGGACCC
CCGGTTCGAGTCTCGGACCGGCCGGACTGCGGCGAACGGGGGTTTGCCTC
CCCGTCATGCAAGACCCCGCTTGCAAATTCCTCCGGAAACAGGGACGAGCC
CCTTTTTTGCTTTTCCCAGATGCATCCGGTGCTGCGGCAGATGCGCCCCCC
TCCTCAGCAGCGGCAAGAGCAAGAGCAGCGGCAGACATGCAGGGCACCCT
CCCCTCCTCCTACCGCGTCAGGAGGGGCGACATCCGCGGTTGACGCGGCA
GCAGATGGTGATTACGAACCCCCGCGGCGCCGGGCCCGGCACTACCTGGA
CTTGGAGGAGGGCGAGGGCCTGGCGCGGCTAGGAGCGCCCTCTCCTGAG
CGGTACCCAAGGGTGCAGCTGAAGCGTGATACGCGTGAGGCGTACGTGCC
GCGGCAGAACCTGTTTCGCGACCGCGAGGGAGAGGAGCCCGAGGAGATG
CGGGATCGAAAGTTCCACGCAGGGCGCGAGCTGCGGCATGGCCTGAATCG
CGAGCGGTTGCTGCGCGAGGAGGACTTTGAGCCCGACGCGCGAACCGGG
ATTAGTCCCGCGCGCGCACACGTGGCGGCCGCCGACCTGGTAACCGCATA
CGAGCAGACGGTGAACCAGGAGATTAACTTTCAAAAAAGCTTTAACAACCAC
GTGCGTACGCTTGTGGCGCGCGAGGAGGTGGCTATAGGACTGATGCATCT
GTGGGACTTTGTAAGCGCGCTGGAGCAAAACCCAAATAGCAAGCCGCTCAT
GGCGCAGCTGTTCCTTATAGTGCAGCACAGCAGGGACAACGAGGCATTCAG
GGATGCGCTGCTAAACATAGTAGAGCCCGAGGGCCGCTGGCTGCTCGATTT
GATAAACATCCTGCAGAGCATAGTGGTGCAGGAGCGCAGCTTGAGCCTGG
CTGACAAGGTGGCCGCCATCAACTATTCCATGCTTAGCCTGGGCAAGTTTTA
CGCCCGCAAGATATACCATACCCCTTACGTTCCCATAGACAAGGAGGTAAA
GATCGAGGGGTTCTACATGCGCATGGCGCTGAAGGTGCTTACCTTGAGCGA
CGACCTGGGCGTTTATCGCAACGAGCGCATCCACAAGGCCGTGAGCGTGA
GCCGGCGGCGCGAGCTCAGCGACCGCGAGCTGATGCACAGCCTGCAAAG
GGCCCTGGCTGGCACGGGCAGCGGCGATAGAGAGGCCGAGTCCTACTTTG
ACGCGGGCGCTGACCTGCGCTGGGCCCCAAGCCGACGCGCCCTGGAGGC
AGCTGGGGCCGGACCTGGGCTGGCGGTGGCACCCGCGCGCGCTGGCAAC
GTCGGCGGCGTGGAGGAATATGACGAGGACGATGAGTACGAGCCAGAGGA
CGGCGAGTACTAAGCGGTGATGTTTCTGATCAGATGATGCAAGACGCAACG
GACCCGGCGGTGCGGGCGGCGCTGCAGAGCCAGCCGTCCGGCCTTAACT
CCACGGACGACTGGCGCCAGGTCATGGACCGCATCATGTCGCTGACTGCG
CGCAATCCTGACGCGTTCCGGCAGCAGCCGCAGGCCAACCGGCTCTCCGC
AATTCTGGAAGCGGTGGTCCCGGCGCGCGCAAACCCCACGCACGAGAAGG
TGCTGGCGATCGTAAACGCGCTGGCCGAAAACAGGGCCATCCGGCCCGAC
GAGGCCGGCCTGGTCTACGACGCGCTGCTTCAGCGCGTGGCTCGTTACAA
CAGCGGCAACGTGCAGACCAACCTGGACCGGCTGGTGGGGGATGTGCGC
GAGGCCGTGGCGCAGCGTGAGCGCGCGCAGCAGCAGGGCAACCTGGGCT
CCATGGTTGCACTAAACGCCTTCCTGAGTACACAGCCCGCCAACGTGCCGC
GGGGACAGGAGGACTACACCAACTTTGTGAGCGCACTGCGGCTAATGGTG
ACTGAGACACCGCAAAGTGAGGTGTACCAGTCTGGGCCAGACTATTTTTTC
CAGACCAGTAGACAAGGCCTGCAGACCGTAAACCTGAGCCAGGCTTTCAAA
AACTTGCAGGGGCTGTGGGGGGTGCGGGCTCCCACAGGCGACCGCGCGA
CCGTGTCTAGCTTGCTGACGCCCAACTCGCGCCTGTTGCTGCTGCTAATAG
CGCCCTTCACGGACAGTGGCAGCGTGTCCCGGGACACATACCTAGGTCAC
TTGCTGACACTGTACCGCGAGGCCATAGGTCAGGCGCATGTGGACGAGCA
TACTTTCCAGGAGATTACAAGTGTCAGCCGCGCGCTGGGGCAGGAGGACA
CGGGCAGCCTGGAGGCAACCCTAAACTACCTGCTGACCAACCGGCGGCAG
AAGATCCCCTCGTTGCACAGTTTAAACAGCGAGGAGGAGCGCATTTTGCGC
TACGTGCAGCAGAGCGTGAGCCTTAACCTGATGCGCGACGGGGTAACGCC
CAGCGTGGCGCTGGACATGACCGCGCGCAACATGGAACCGGGCATGTATG
CCTCAAACCGGCCGTTTATCAACCGCCTAATGGACTACTTGCATCGCGCGG
CCGCCGTGAACCCCGAGTATTTCACCAATGCCATCTTGAACCCGCACTGGC
TACCGCCCCCTGGTTTCTACACCGGGGGATTCGAGGTGCCCGAGGGTAAC
GATGGATTCCTCTGGGACGACATAGACGACAGCGTGTTTTCCCCGCAACCG
CAGACCCTGCTAGAGTTGCAACAGCGCGAGCAGGCAGAGGCGGCGCTGCG
AAAGGAAAGCTTCCGCAGGCCAAGCAGCTTGTCCGATCTAGGCGCTGCGG
CCCCGCGGTCAGATGCTAGTAGCCCATTTCCAAGCTTGATAGGGTCTCTTA
CCAGCACTCGCACCACCCGCCCGCGCCTGCTGGGCGAGGAGGAGTACCTA
AACAACTCGCTGCTGCAGCCGCAGCGCGAAAAAAACCTGCCTCCGGCATTT
CCCAACAACGGGATAGAGAGCCTAGTGGACAAGATGAGTAGATGGAAGAC
GTACGCGCAGGAGCACAGGGACGTGCCAGGCCCGCGCCCGCCCACCCGT
CGTCAAAGGCACGACCGTCAGCGGGGTCTGGTGTGGGAGGACGATGACTC
GGCAGACGACAGCAGCGTCCTGGATTTGGGAGGGAGTGGCAACCCGTTTG
CGCACCTTCGCCCCAGGCTGGGGAGAATGTTTTAAAAAAAAAAAAGCATGA
TGCAAAATAAAAAACTCACCAAGGCCATGGCACCGAGCGTTGGTTTTCTTGT
ATTCCCCTTAGTATGCGGCGCGCGGCGATGTATGAGGAAGGTCCTCCTCCC
TCCTACGAGAGTGTGGTGAGCGCGGCGCCAGTGGCGGCGGCGCTGGGTT
CTCCCTTCGATGCTCCCCTGGACCCGCCGTTTGTGCCTCCGCGGTACCTGC
GGCCTACCGGGGGGAGAAACAGCATCCGTTACTCTGAGTTGGCACCCCTAT
TCGACACCACCCGTGTGTACCTGGTGGACAACAAGTCAACGGATGTGGCAT
CCCTGAACTACCAGAACGACCACAGCAACTTTCTGACCACGGTCATTCAAAA
CAATGACTACAGCCCGGGGGAGGCAAGCACACAGACCATCAATCTTGACGA
CCGGTCGCACTGGGGCGGCGACCTGAAAACCATCCTGCATACCAACATGC
CAAATGTGAACGAGTTCATGTTTACCAATAAGTTTAAGGCGCGGGTGATGGT
GTCGCGCTTGCCTACTAAGGACAATCAGGTGGAGCTGAAATACGAGTGGGT
GGAGTTCACGCTGCCCGAGGGCAACTACTCCGAGACCATGACCATAGACCT
TATGAACAACGCGATCGTGGAGCACTACTTGAAAGTGGGCAGACAGAACGG
GGTTCTGGAAAGCGACATCGGGGTAAAGTTTGACACCCGCAACTTCAGACT
GGGGTTTGACCCCGTCACTGGTCTTGTCATGCCTGGGGTATATACAAACGA
AGCCTTCCATCCAGACATCATTTTGCTGCCAGGATGCGGGGTGGACTTCAC
CCACAGCCGCCTGAGCAACTTGTTGGGCATCCGCAAGCGGCAACCCTTCCA
GGAGGGCTTTAGGATCACCTACGATGATCTGGAGGGTGGTAACATTCCCGC
ACTGTTGGATGTGGACGCCTACCAGGCGAGCTTGAAAGATGACACCGAACA
GGGCGGGGGTGGCGCAGGCGGCAGCAACAGCAGTGGCAGCGGCGCGGA
AGAGAACTCCAACGCGGCAGCCGCGGCAATGCAGCCGGTGGAGGACATGA
ACGATCATGCCATTCGCGGCGACACCTTTGCCACACGGGCTGAGGAGAAG
CGCGCTGAGGCCGAAGCAGCGGCCGAAGCTGCCGCCCCCGCTGCGCAAC
CCGAGGTCGAGAAGCCTCAGAAGAAACCGGTGATCAAACCCCTGACAGAG
GACAGCAAGAAACGCAGTTACAACCTAATAAGCAATGACAGCACCTTCACC
CAGTACCGCAGCTGGTACCTTGCATACAACTACGGCGACCCTCAGACCGGA
ATCCGCTCATGGACCCTGCTTTGCACTCCTGACGTAACCTGCGGCTCGGAG
CAGGTCTACTGGTCGTTGCCAGACATGATGCAAGACCCCGTGACCTTCCGC
TCCACGCGCCAGATCAGCAACTTTCCGGTGGTGGGCGCCGAGCTGTTGCC
CGTGCACTCCAAGAGCTTCTACAACGACCAGGCCGTCTACTCCCAACTCAT
CCGCCAGTTTACCTCTCTGACCCACGTGTTCAATCGCTTTCCCGAGAACCA
GATTTTGGCGCGCCCGCCAGCCCCCACCATCACCACCGTCAGTGAAAACGT
TCCTGCTCTCACAGATCACGGGACGCTACCGCTGCGCAACAGCATCGGAG
GAGTCCAGCGAGTGACCATTACTGACGCCAGACGCCGCACCTGCCCCTAC
GTTTACAAGGCCCTGGGCATAGTCTCGCCGCGCGTCCTATCGAGCCGCACT
TTTTGAGCAAGCATGTCCATCCTTATATCGCCCAGCAATAACACAGGCTGGG
GCCTGCGCTTCCCAAGCAAGATGTTTGGCGGGGCCAAGAAGCGCTCCGAC
CAACACCCAGTGCGCGTGCGCGGGCACTACCGCGCGCCCTGGGGCGCGC
ACAAACGCGGCCGCACTGGGCGCACCACCGTCGATGACGCCATCGACGCG
GTGGTGGAGGAGGCGCGCAACTACACGCCCACGCCGCCACCAGTGTCCAC
AGTGGACGCGGCCATTCAGACCGTGGTGCGCGGAGCCCGGCGCTATGCTA
AAATGAAGAGACGGCGGAGGCGCGTAGCACGTCGCCACCGCCGCCGACC
CGGCACTGCCGCCCAACGCGCGGCGGCGGCCCTGCTTAACCGCGCACGT
CGCACCGGCCGACGGGCGGCCATGCGGGCCGCTCGAAGGCTGGCCGCGG
GTATTGTCACTGTGCCCCCCAGGTCCAGGCGACGAGCGGCCGCCGCAGCA
GCCGCGGCCATTAGTGCTATGACTCAGGGTCGCAGGGGCAACGTGTATTG
GGTGCGCGACTCGGTTAGCGGCCTGCGCGTGCCCGTGCGCACCCGCCCC
CCGCGCAACTAGATTGCAAGAAAAAACTACTTAGACTCGTACTGTTGTATGT
ATCCAGCGGCGGCGGCGCGCAACGAAGCTATGTCCAAGCGCAAAATCAAA
GAAGAGATGCTCCAGGTCATCGCGCCGGAGATCTATGGCCCCCCGAAGAA
GGAAGAGCAGGATTACAAGCCCCGAAAGCTAAAGCGGGTCAAAAAGAAAAA
GAAAGATGATGATGATGAACTTGACGACGAGGTGGAACTGCTGCACGCTAC
CGCGCCCAGGCGACGGGTACAGTGGAAAGGTCGACGCGTAAAACGTGTTT
TGCGACCCGGCACCACCGTAGTCTTTACGCCCGGTGAGCGCTCCACCCGC
ACCTACAAGCGCGTGTATGATGAGGTGTACGGCGACGAGGACCTGCTTGA
GCAGGCCAACGAGCGCCTCGGGGAGTTTGCCTACGGAAAGCGGCATAAGG
ACATGCTGGCGTTGCCGCTGGACGAGGGCAACCCAACACCTAGCCTAAAG
CCCGTAACACTGCAGCAGGTGCTGCCCGCGCTTGCACCGTCCGAAGAAAA
GCGCGGCCTAAAGCGCGAGTCTGGTGACTTGGCACCCACCGTGCAGCTGA
TGGTACCCAAGCGCCAGCGACTGGAAGATGTCTTGGAAAAAATGACCGTGG
AACCTGGGCTGGAGCCCGAGGTCCGCGTGCGGCCAATCAAGCAGGTGGC
GCCGGGACTGGGCGTGCAGACCGTGGACGTTCAGATACCCACTACCAGTA
GCACCAGTATTGCCACCGCCACAGAGGGCATGGAGACACAAACGTCCCCG
GTTGCCTCAGCGGTGGCGGATGCCGCGGTGCAGGCGGTCGCTGCGGCCG
CGTCCAAGACCTCTACGGAGGTGCAAACGGACCCGTGGATGTTTCGCGTTT
CAGCCCCCCGGCGCCCGCGCGGTTCGAGGAAGTACGGCGCCGCCAGCGC
GCTACTGCCCGAATATGCCCTACATCCTTCCATTGCGCCTACCCCCGGCTA
TCGTGGCTACACCTACCGCCCCAGAAGACGAGCAACTACCCGACGCCGAA
CCACCACTGGAACCCGCCGCCGCCGTCGCCGTCGCCAGCCCGTGCTGGC
CCCGATTTCCGTGCGCAGGGTGGCTCGCGAAGGAGGCAGGACCCTGGTGC
TGCCAACAGCGCGCTACCACCCCAGCATCGTTTAAAAGCCGGTCTTTGTGG
TTCTTGCAGATATGGCCCTCACCTGCCGCCTCCGTTTCCCGGTGCCGGGAT
TCCGAGGAAGAATGCACCGTAGGAGGGGCATGGCCGGCCACGGCCTGAC
GGGCGGCATGCGTCGTGCGCACCACCGGCGGCGGCGCGCGTCGCACCGT
CGCATGCGCGGCGGTATCCTGCCCCTCCTTATTCCACTGATCGCCGCGGC
GATTGGCGCCGTGCCCGGAATTGCATCCGTGGCCTTGCAGGCGCAGAGAC
ACTGATTAAAAACAAGTTGCATGTGGAAAAATCAAAATAAAAAGTCTGGACT
CTCACGCTCGCTTGGTCCTGTAACTATTTTGTAGAATGGAAGACATCAACTT
TGCGTCTCTGGCCCCGCGACACGGCTCGCGCCCGTTCATGGGAAACTGGC
AAGATATCGGCACCAGCAATATGAGCGGTGGCGCCTTCAGCTGGGGCTCG
CTGTGGAGCGGCATTAAAAATTTCGGTTCCACCGTTAAGAACTATGGCAGCA
AGGCCTGGAACAGCAGCACAGGCCAGATGCTGAGGGATAAGTTGAAAGAG
CAAAATTTCCAACAAAAGGTGGTAGATGGCCTGGCCTCTGGCATTAGCGGG
GTGGTGGACCTGGCCAACCAGGCAGTGCAAAATAAGATTAACAGTAAGCTT
GATCCCCGCCCTCCCGTAGAGGAGCCTCCACCGGCCGTGGAGACAGTGTC
TCCAGAGGGGCGTGGCGAAAAGCGTCCGCGCCCCGACAGGGAAGAAACTC
TGGTGACGCAAATAGACGAGCCTCCCTCGTACGAGGAGGCACTAAAGCAAG
GCCTGCCCACCACCCGTCCCATCGCGCCCATGGCTACCGGAGTGCTGGGC
CAGCACACACCCGTAACGCTGGACCTGCCTCCCCCCGCCGACACCCAGCA
GAAACCTGTGCTGCCAGGCCCGACCGCCGTTGTTGTAACCCGTCCTAGCC
GCGCGTCCCTGCGCCGCGCCGCCAGCGGTCCGCGATCGTTGCGGCCCGT
AGCCAGTGGCAACTGGCAAAGCACACTGAACAGCATCGTGGGTCTGGGGG
TGCAATCCCTGAAGCGCCGACGATGCTTCTGAATAGCTAACGTGTCGTATG
TGTGTCATGTATGCGTCCATGTCGCCGCCAGAGGAGCTGCTGAGCCGCCG
CGCGCCCGCTTTCCAAGATGGCTACCCCTTCGATGATGCCGCAGTGGTCTT
ACATGCACATCTCGGGCCAGGACGCCTCGGAGTACCTGAGCCCCGGGCTG
GTGCAGTTTGCCCGCGCCACCGAGACGTACTTCAGCCTGAATAACAAGTTT
AGAAACCCCACGGTGGCGCCTACGCACGACGTGACCACAGACCGGTCCCA
GCGTTTGACGCTGCGGTTCATCCCTGTGGACCGTGAGGATACTGCGTACTC
GTACAAGGCGCGGTTCACCCTAGCTGTGGGTGATAACCGTGTGCTGGACAT
GGCTTCCACGTACTTTGACATCCGCGGCGTGCTGGACAGGGGCCCTACTTT
TAAGCCCTACTCTGGCACTGCCTACAACGCCCTGGCTCCCAAGGGTGCCCC
AAATCCTTGCGAATGGGATGAAGCTGCTACTGCTCTTGAAATAAACCTAGAA
GAAGAGGACGATGACAACGAAGACGAAGTAGACGAGCAAGCTGAGCAGCA
AAAAACTCACGTATTTGGGCAGGCGCCTTATTCTGGTATAAATATTACAAAG
GAGGGTATTCAAATAGGTGTCGAAGGTCAAACACCTAAATATGCCGATAAAA
CATTTCAACCTGAACCTCAAATAGGAGAATCTCAGTGGTACGAAACTGAAAT
TAATCATGCAGCTGGGAGAGTCCTTAAAAAGACTACCCCAATGAAACCATGT
TACGGTTCATATGCAAAACCCACAAATGAAAATGGAGGGCAAGGCATTCTTG
TAAAGCAACAAAATGGAAAGCTAGAAAGTCAAGTGGAAATGCAATTTTTCTC
AACTACTGAGGCGACCGCAGGCAATGGTGATAACTTGACTCCTAAAGTGGT
ATTGTACAGTGAAGATGTAGATATAGAAACCCCAGACACTCATATTTCTTACA
TGCCCACTATTAAGGAAGGTAACTCACGAGAACTAATGGGCCAACAATCTAT
GCCCAACAGGCCTAATTACATTGCTTTTAGGGACAATTTTATTGGTCTAATGT
ATTACAACAGCACGGGTAATATGGGTGTTCTGGCGGGCCAAGCATCGCAGT
TGAATGCTGTTGTAGATTTGCAAGACAGAAACACAGAGCTTTCATACCAGCT
TTTGCTTGATTCCATTGGTGATAGAACCAGGTACTTTTCTATGTGGAATCAG
GCTGTTGACAGCTATGATCCAGATGTTAGAATTATTGAAAATCATGGAACTG
AAGATGAACTTCCAAATTACTGCTTTCCACTGGGAGGTGTGATTAATACAGA
GACTCTTACCAAGGTAAAACCTAAAACAGGTCAGGAAAATGGATGGGAAAAA
GATGCTACAGAATTTTCAGATAAAAATGAAATAAGAGTTGGAAATAATTTTGC
CATGGAAATCAATCTAAATGCCAACCTGTGGAGAAATTTCCTGTACTCCAAC
ATAGCGCTGTATTTGCCCGACAAGCTAAAGTACAGTCCTTCCAACGTAAAAA
TTTCTGATAACCCAAACACCTACGACTACATGAACAAGCGAGTGGTGGCTCC
CGGGTTAGTGGACTGCTACATTAACCTTGGAGCACGCTGGTCCCTTGACTA
TATGGACAACGTCAACCCATTTAACCACCACCGCAATGCTGGCCTGCGCTA
CCGCTCAATGTTGCTGGGCAATGGTCGCTATGTGCCCTTCCACATCCAGGT
GCCTCAGAAGTTCTTTGCCATTAAAAACCTCCTTCTCCTGCCGGGCTCATAC
ACCTACGAGTGGAACTTCAGGAAGGATGTTAACATGGTTCTGCAGAGCTCC
CTAGGAAATGACCTAAGGGTTGACGGAGCCAGCATTAAGTTTGATAGCATTT
GCCTTTACGCCACCTTCTTCCCCATGGCCCACAACACCGCCTCCACGCTTG
AGGCCATGCTTAGAAACGACACCAACGACCAGTCCTTTAACGACTATCTCTC
CGCCGCCAACATGCTCTACCCTATACCCGCCAACGCTACCAACGTGCCCAT
ATCCATCCCCTCCCGCAACTGGGCGGCTTTCCGCGGCTGGGCCTTCACGC
GCCTTAAGACTAAGGAAACCCCATCACTGGGCTCGGGCTACGACCCTTATT
ACACCTACTCTGGCTCTATACCCTACCTAGATGGAACCTTTTACCTCAACCA
CACCTTTAAGAAGGTGGCCATTACCTTTGACTCTTCTGTCAGCTGGCCTGGC
AATGACCGCCTGCTTACCCCCAACGAGTTTGAAATTAAGCGCTCAGTTGAC
GGGGAGGGTTACAACGTTGCCCAGTGTAACATGACCAAAGACTGGTTCCTG
GTACAAATGCTAGCTAACTACAACATTGGCTACCAGGGCTTCTATATCCCAG
AGAGCTACAAGGACCGCATGTACTCCTTCTTTAGAAACTTCCAGCCCATGAG
CCGTCAGGTGGTGGATGATACTAAATACAAGGACTACCAACAGGTGGGCAT
CCTACACCAACACAACAACTCTGGATTTGTTGGCTACCTTGCCCCCACCATG
CGCGAAGGACAGGCCTACCCTGCTAACTTCCCCTATCCGCTTATAGGCAAG
ACCGCAGTTGACAGCATTACCCAGAAAAAGTTTCTTTGCGATCGCACCCTTT
GGCGCATCCCATTCTCCAGTAACTTTATGTCCATGGGCGCACTCACAGACC
TGGGCCAAAACCTTCTCTACGCCAACTCCGCCCACGCGCTAGACATGACTT
TTGAGGTGGATCCCATGGACGAGCCCACCCTTCTTTATGTTTTGTTTGAAGT
CTTTGACGTGGTCCGTGTGCACCGGCCGCACCGCGGCGTCATCGAAACCG
TGTACCTGCGCACGCCCTTCTCGGCCGGCAACGCCACAACATAAAGAAGCA
AGCAACATCAACAACAGCTGCCGCCATGGGCTCCAGTGAGCAGGAACTGAA
AGCCATTGTCAAAGATCTTGGTTGTGGGCCATATTTTTTGGGCACCTATGAC
AAGCGCTTTCCAGGCTTTGTTTCTCCACACAAGCTCGCCTGCGCCATAGTCA
ATACGGCCGGTCGCGAGACTGGGGGCGTACACTGGATGGCCTTTGCCTGG
AACCCGCACTCAAAAACATGCTACCTCTTTGAGCCCTTTGGCTTTTCTGACC
AGCGACTCAAGCAGGTTTACCAGTTTGAGTACGAGTCACTCCTGCGCCGTA
GCGCCATTGCTTCTTCCCCCGACCGCTGTATAACGCTGGAAAAGTCCACCC
AAAGCGTACAGGGGCCCAACTCGGCCGCCTGTGGACTATTCTGCTGCATGT
TTCTCCACGCCTTTGCCAACTGGCCCCAAACTCCCATGGATCACAACCCCA
CCATGAACCTTATTACCGGGGTACCCAACTCCATGCTCAACAGTCCCCAGG
TACAGCCCACCCTGCGTCGCAACCAGGAACAGCTCTACAGCTTCCTGGAGC
GCCACTCGCCCTACTTCCGCAGCCACAGTGCGCAGATTAGGAGCGCCACTT
CTTTTTGTCACTTGAAAAACATGTAAAAATAATGTACTAGAGACACTTTCAAT
AAAGGCAAATGCTTTTATTTGTACACTCTCGGGTGATTATTTACCCCCACCCT
TGCCGTCTGCGCCGTTTAAAAATCAAAGGGGTTCTGCCGCGCATCGCTATG
CGCCACTGGCAGGGACACGTTGCGATACTGGTGTTTAGTGCTCCACTTAAA
CTCAGGCACAACCATCCGCGGCAGCTCGGTGAAGTTTTCACTCCACAGGCT
GCGCACCATCACCAACGCGTTTAGCAGGTCGGGCGCCGATATCTTGAAGTC
GCAGTTGGGGCCTCCGCCCTGCGCGCGCGAGTTGCGATACACAGGGTTGC
AGCACTGGAACACTATCAGCGCCGGGTGGTGCACGCTGGCCAGCACGCTC
TTGTCGGAGATCAGATCCGCGTCCAGGTCCTCCGCGTTGCTCAGGGCGAA
CGGAGTCAACTTTGGTAGCTGCCTTCCCAAAAAGGGCGCGTGCCCAGGCTT
TGAGTTGCACTCGCACCGTAGTGGCATCAAAAGGTGACCGTGCCCGGTCTG
GGCGTTAGGATACAGCGCCTGCATAAAAGCCTTGATCTGCTTAAAAGCCAC
CTGAGCCTTTGCGCCTTCAGAGAAGAACATGCCGCAAGACTTGCCGGAAAA
CTGATTGGCCGGACAGGCCGCGTCGTGCACGCAGCACCTTGCGTCGGTGT
TGGAGATCTGCACCACATTTCGGCCCCACCGGTTCTTCACGATCTTGGCCT
TGCTAGACTGCTCCTTCAGCGCGCGCTGCCCGTTTTCGCTCGTCACATCCA
TTTCAATCACGTGCTCCTTATTTATCATAATGCTTCCGTGTAGACACTTAAGC
TCGCCTTCGATCTCAGCGCAGCGGTGCAGCCACAACGCGCAGCCCGTGGG
CTCGTGATGCTTGTAGGTCACCTCTGCAAACGACTGCAGGTACGCCTGCAG
GAATCGCCCCATCATCGTCACAAAGGTCTTGTTGCTGGTGAAGGTCAGCTG
CAACCCGCGGTGCTCCTCGTTCAGCCAGGTCTTGCATACGGCCGCCAGAG
CTTCCACTTGGTCAGGCAGTAGTTTGAAGTTCGCCTTTAGATCGTTATCCAC
GTGGTACTTGTCCATCAGCGCGCGCGCAGCCTCCATGCCCTTCTCCCACGC
AGACACGATCGGCACACTCAGCGGGTTCATCACCGTAATTTCACTTTCCGCT
TCGCTGGGCTCTTCCTCTTCCTCTTGCGTCCGCATACCACGCGCCACTGGG
TCGTCTTCATTCAGCCGCCGCACTGTGCGCTTACCTCCTTTGCCATGCTTGA
TTAGCACCGGTGGGTTGCTGAAACCCACCATTTGTAGCGCCACATCTTCTCT
TTCTTCCTCGCTGTCCACGATTACCTCTGGTGATGGCGGGCGCTCGGGCTT
GGGAGAAGGGCGCTTCTTTTTCTTCTTGGGCGCAATGGCCAAATCCGCCGC
CGAGGTCGATGGCCGCGGGCTGGGTGTGCGCGGCACCAGCGCGTCTTGT
GATGAGTCTTCCTCGTCCTCGGACTCGATACGCCGCCTCATCCGCTTTTTTG
GGGGCGCCCGGGGAGGCGGCGGCGACGGGGACGGGGACGACACGTCCT
CCATGGTTGGGGGACGTCGCGCCGCACCGCGTCCGCGCTCGGGGGTGGT
TTCGCGCTGCTCCTCTTCCCGACTGGCCATTTCCTTCTCCTATAGGCAGAAA
AAGATCATGGAGTCAGTCGAGAAGAAGGACAGCCTAACCGCCCCCTCTGAG
TTCGCCACCACCGCCTCCACCGATGCCGCCAACGCGCCTACCACCTTCCCC
GTCGAGGCACCCCCGCTTGAGGAGGAGGAAGTGATTATCGAGCAGGACCC
AGGTTTTGTAAGCGAAGACGACGAGGACCGCTCAGTACCAACAGAGGATAA
AAAGCAAGACCAGGACAACGCAGAGGCAAACGAGGAACAAGTCGGGCGGG
GGGACGAAAGGCATGGCGACTACCTAGATGTGGGAGACGACGTGCTGTTG
AAGCATCTGCAGCGCCAGTGCGCCATTATCTGCGACGCGTTGCAAGAGCG
CAGCGATGTGCCCCTCGCCATAGCGGATGTCAGCCTTGCCTACGAACGCCA
CCTATTCTCACCGCGCGTACCCCCCAAACGCCAAGAAAACGGCACATGCGA
GCCCAACCCGCGCCTCAACTTCTACCCCGTATTTGCCGTGCCAGAGGTGCT
TGCCACCTATCACATCTTTTTCCAAAACTGCAAGATACCCCTATCCTGCCGT
GCCAACCGCAGCCGAGCGGACAAGCAGCTGGCCTTGCGGCAGGGCGCTG
TCATACCTGATATCGCCTCGCTCAACGAAGTGCCAAAAATCTTTGAGGGTCT
TGGACGCGACGAGAAGCGCGCGGCAAACGCTCTGCAACAGGAAAACAGCG
AAAATGAAAGTCACTCTGGAGTGTTGGTGGAACTCGAGGGTGACAACGCGC
GCCTAGCCGTACTAAAACGCAGCATCGAGGTCACCCACTTTGCCTACCCGG
CACTTAACCTACCCCCCAAGGTCATGAGCACAGTCATGAGTGAGCTGATCG
TGCGCCGTGCGCAGCCCCTGGAGAGGGATGCAAATTTGCAAGAACAAACA
GAGGAGGGCCTACCCGCAGTTGGCGACGAGCAGCTAGCGCGCTGGCTTCA
AACGCGCGAGCCTGCCGACTTGGAGGAGCGACGCAAACTAATGATGGCCG
CAGTGCTCGTTACCGTGGAGCTTGAGTGCATGCAGCGGTTCTTTGCTGACC
CGGAGATGCAGCGCAAGCTAGAGGAAACATTGCACTACACCTTTCGACAGG
GCTACGTACGCCAGGCCTGCAAGATCTCCAACGTGGAGCTCTGCAACCTGG
TCTCCTACCTTGGAATTTTGCACGAAAACCGCCTTGGGCAAAACGTGCTTCA
TTCCACGCTCAAGGGCGAGGCGCGCCGCGACTACGTCCGCGACTGCGTTT
ACTTATTTCTATGCTACACCTGGCAGACGGCCATGGGCGTTTGGCAGCAGT
GCTTGGAGGAGTGCAACCTCAAGGAGCTGCAGAAACTGCTAAAGCAAAACT
TGAAGGACCTATGGACGGCCTTCAACGAGCGCTCCGTGGCCGCGCACCTG
GCGGACATCATTTTCCCCGAACGCCTGCTTAAAACCCTGCAACAGGGTCTG
CCAGACTTCACCAGTCAAAGCATGTTGCAGAACTTTAGGAACTTTATCCTAG
AGCGCTCAGGAATCTTGCCCGCCACCTGCTGTGCACTTCCTAGCGACTTTG
TGCCCATTAAGTACCGCGAATGCCCTCCGCCGCTTTGGGGCCACTGCTACC
TTCTGCAGCTAGCCAACTACCTTGCCTACCACTCTGACATAATGGAAGACGT
GAGCGGTGACGGTCTACTGGAGTGTCACTGTCGCTGCAACCTATGCACCCC
GCACCGCTCCCTGGTTTGCAATTCGCAGCTGCTTAACGAAAGTCAAATTATC
GGTACCTTTGAGCTGCAGGGTCCCTCGCCTGACGAAAAGTCCGCGGCTCC
GGGGTTGAAACTCACTCCGGGGCTGTGGACGTCGGCTTACCTTCGCAAATT
TGTACCTGAGGACTACCACGCCCACGAGATTAGGTTCTACGAAGACCAATC
CCGCCCGCCAAATGCGGAGCTTACCGCCTGCGTCATTACCCAGGGCCACA
TTCTTGGCCAATTGCAAGCCATCAACAAAGCCCGCCAAGAGTTTCTGCTACG
AAAGGGACGGGGGGTTTACTTGGACCCCCAGTCCGGCGAGGAGCTCAACC
CAATCCCCCCGCCGCCGCAGCCCTATCAGCAGCAGCCGCGGGCCCTTGCT
TCCCAGGATGGCACCCAAAAAGAAGCTGCAGCTGCCGCCGCCACCCACGG
ACGAGGAGGAATACTGGGACAGTCAGGCAGAGGAGGTTTTGGACGAGGAG
GAGGAGGACATGATGGAAGACTGGGAGAGCCTAGACGAGGAAGCTTCCGA
GGTCGAAGAGGTGTCAGACGAAACACCGTCACCCTCGGTCGCATTCCCCTC
GCCGGCGCCCCAGAAATCGGCAACCGGTTCCAGCATGGCTACAACCTCCG
CTCCTCAGGCGCCGCCGGCACTGCCCGTTCGCCGACCCAACCGTAGATGG
GACACCACTGGAACCAGGGCCGGTAAGTCCAAGCAGCCGCCGCCGTTAGC
CCAAGAGCAACAACAGCGCCAAGGCTACCGCTCATGGCGCGGGCACAAGA
ACGCCATAGTTGCTTGCTTGCAAGACTGTGGGGGCAACATCTCCTTCGCCC
GCCGCTTTCTTCTCTACCATCACGGCGTGGCCTTCCCCCGTAACATCCTGC
ATTACTACCGTCATCTCTACAGCCCATACTGCACCGGCGGCAGCGGCAGCG
GCAGCAACAGCAGCGGCCACACAGAAGCAAAGGCGACCGGATAGCAAGAC
TCTGACAAAGCCCAAGAAATCCACAGCGGCGGCAGCAGCAGGAGGAGGAG
CGCTGCGTCTGGCGCCCAACGAACCCGTATCGACCCGCGAGCTTAGAAAC
AGGATTTTTCCCACTCTGTATGCTATATTTCAACAGAGCAGGGGCCAAGAAC
AAGAGCTGAAAATAAAAAACAGGTCTCTGCGATCCCTCACCCGCAGCTGCC
TGTATCACAAAAGCGAAGATCAGCTTCGGCGCACGCTGGAAGACGCGGAG
GCTCTCTTCAGTAAATACTGCGCGCTGACTCTTAAGGACTAGTTTCGCGCCC
TTTCTCAAATTTAAGCGCGAAAACTACGTCATCTCCAGCGGCCACACCCGG
CGCCAGCACCTGTCGTCAGCGCCATTATGAGCAAGGAAATTCCCACGCCCT
ACATGTGGAGTTACCAGCCACAAATGGGACTTGCGGCTGGAGCTGCCCAAG
ACTACTCAACCCGAATAAACTACATGAGCGCGGGACCCCACATGATATCCC
GGGTCAACGGAATCCGCGCCCACCGAAACCGAATTCTCTTGGAACAGGCG
GCTATTACCACCACACCTCGTAATAACCTTAATCCCCGTAGTTGGCCCGCTG
CCCTGGTGTACCAGGAAAGTCCCGCTCCCACCACTGTGGTACTTCCCAGAG
ACGCCCAGGCCGAAGTTCAGATGACTAACTCAGGGGCGCAGCTTGCGGGC
GGCTTTCGTCACAGGGTGCGGTCGCCCGGGCAGGGTATAACTCACCTGAC
AATCAGAGGGCGAGGTATTCAGCTCAACGACGAGTCGGTGAGCTCCTCGCT
TGGTCTCCGTCCGGACGGGACATTTCAGATCGGCGGCGCCGGCCGTCCTT
CATTCACGCCTCGTCAGGCAATCCTAACTCTGCAGACCTCGTCCTCTGAGC
CGCGCTCTGGAGGCATTGGAACTCTGCAATTTATTGAGGAGTTTGTGCCAT
CGGTCTACTTTAACCCCTTCTCGGGACCTCCCGGCCACTATCCGGATCAATT
TATTCCTAACTTTGACGCGGTAAAGGACTCGGCGGACGGCTACGACTGAAT
GTTAAGTGGAGAGGCAGAGCAACTGCGCCTGAAACACCTGGTCCACTGTCG
CCGCCACAAGTGCTTTGCCCGCGACTCCGGTGAGTTTTGCTACTTTGAATT
GCCCGAGGATCATATCGAGGGCCCGGCGCACGGCGTCCGGCTTACCGCCC
AGGGAGAGCTTGCCCGTAGCCTGATTCGGGAGTTTACCCAGCGCCCCCTG
CTAGTTGAGCGGGACAGGGGACCCTGTGTTCTCACTGTGATTTGCAACTGT
CCTAACCTTGGATTACATCAAGATCTTTGTTGCCATCTCTGTGCTGAGTATAA
TAAATACAGAAATTAAAATATACTGGGGCTCCTATCGCCATCCTGTAAACGC
CACCGTCTTCACCCGCCCAAGCAAACCAAGGCGAACCTTACCTGGTACTTT
TAACATCTCTCCCTCTGTGATTTACAACAGTTTCAACCCAGACGGAGTGAGT
CTACGAGAGAACCTCTCCGAGCTCAGCTACTCCATCAGAAAAAACACCACC
CTCCTTACCTGCCGGGAACGTACGAGTGCGTCACCGGCCGCTGCACCACA
CCTACCGCCTGACCGTAAACCAGACTTTTTCCGGACAGACCTCAATAACTCT
GTTTACCAGAACAGGAGGTGAGCTTAGAAAACCCTTAGGGTATTAGGCCAA
AGGCGCAGCTACTGTGGGGTTTATGAACAATTCAAGCAACTCTACGGGCTA
TTCTAATTCAGGTTTCTCTAGAAATGGACGGAATTATTACAGAGCAGCGCCT
GCTAGAAAGACGCAGGGCAGCGGCCGAGCAACAGCGCATGAATCAAGAGC
TCCAAGACATGGTTAACTTGCACCAGTGCAAAAGGGGTATCTTTTGTCTGGT
AAAGCAGGCCAAAGTCACCTACGACAGTAATACCACCGGACACCGCCTTAG
CTACAAGTTGCCAACCAAGCGTCAGAAATTGGTGGTCATGGTGGGAGAAAA
GCCCATTACCATAACTCAGCACTCGGTAGAAACCGAAGGCTGCATTCACTC
ACCTTGTCAAGGACCTGAGGATCTCTGCACCCTTATTAAGACCCTGTGCGG
TCTCAAAGATCTTATTCCCTTTAACTAATAAAAAAAAATAATAAAGCATCACTT
ACTTAAAATCAGTTAGCAAATTTCTGTCCAGTTTATTCAGCAGCACCTCCTTG
CCCTCCTCCCAGCTCTGGTATTGCAGCTTCCTCCTGGCTGCAAACTTTCTCC
ACAATCTAAATGGAATGTCAGTTTCCTCCTGTTCCTGTCCATCCGCACCCAC
TATCTTCATGTTGTTGCAGATGAAGCGCGCAAGACCGTCTGAAGATACCTTC
AACCCCGTGTATCCATATGACACGGAAACCGGTCCTCCAACTGTGCCTTTTC
TTACTCCTCCCTTTGTATCCCCCAATGGGTTTCAAGAGAGTCCCCCTGGGGT
ACTCTCTTTGCGCCTATCCGAACCTCTAGTTACCTCCAATGGCATGCTTGCG
CTCAAAATGGGCAACGGCCTCTCTCTGGACGAGGCCGGCAACCTTACCTCC
CAAAATGTAACCACTGTGAGCCCACCTCTCAAAAAAACCAAGTCAAACATAA
ACCTGGAAATATCTGCACCCCTCACAGTTACCTCAGAAGCCCTAACTGTGG
CTGCCGCCGCACCTCTAATGGTCGCGGGCAACACACTCACCATGCAATCAC
AGGCCCCGCTAACCGTGCACGACTCCAAACTTAGCATTGCCACCCAAGGAC
CCCTCACAGTGTCAGAAGGAAAGCTAGCCCTGCAAACATCAGGCCCCCTCA
CCACCACCGATAGCAGTACCCTTACTATCACTGCCTCACCCCCTCTAACTAC
TGCCACTGGTAGCTTGGGCATTGACTTGAAAGAGCCCATTTATACACAAAAT
GGAAAACTAGGACTAAAGTACGGGGCTCCTTTGCATGTAACAGACGACCTA
AACACTTTGACCGTAGCAACTGGTCCAGGTGTGACTATTAATAATACTTCCT
TGCAAACTAAAGTTACTGGAGCCTTGGGTTTTGATTCACAAGGCAATATGCA
ACTTAATGTAGCAGGAGGACTAAGGATTGATTCTCAAAACAGACGCCTTATA
CTTGATGTTAGTTATCCGTTTGATGCTCAAAACCAACTAAATCTAAGACTAGG
ACAGGGCCCTCTTTTTATAAACTCAGCCCACAACTTGGATATTAACTACAAC
AAAGGCCTTTACTTGTTTACAGCTTCAAACAATTCCAAAAAGCTTGAGGTTAA
CCTAAGCACTGCCAAGGGGTTGATGTTTGACGCTACAGCCATAGCCATTAAT
GCAGGAGATGGGCTTGAATTTGGTTCACCTAATGCACCAAACACAAATCCC
CTCAAAACAAAAATTGGCCATGGCCTAGAATTTGATTCAAACAAGGCTATGG
TTCCTAAACTAGGAACTGGCCTTAGTTTTGACAGCACAGGTGCCATTACAGT
AGGAAACAAAAATAATGATAAGCTAACTTTGTGGACCACACCAGCTCCATCT
CCTAACTGTAGACTAAATGCAGAGAAAGATGCTAAACTCACTTTGGTCTTAA
CAAAATGTGGCAGTCAAATACTTGCTACAGTTTCAGTTTTGGCTGTTAAAGG
CAGTTTGGCTCCAATATCTGGAACAGTTCAAAGTGCTCATCTTATTATAAGAT
TTGACGAAAATGGAGTGCTACTAAACAATTCCTTCCTGGACCCAGAATATTG
GAACTTTAGAAATGGAGATCTTACTGAAGGCACAGCCTATACAAACGCTGTT
GGATTTATGCCTAACCTATCAGCTTATCCAAAATCTCACGGTAAAACTGCCA
AAAGTAACATTGTCAGTCAAGTTTACTTAAACGGAGACAAAACTAAACCTGTA
ACACTAACCATTACACTAAACGGTACACAGGAAACAGGAGACACAACTCCAA
GTGCATACTCTATGTCATTTTCATGGGACTGGTCTGGCCACAACTACATTAA
TGAAATATTTGCCACATCCTCTTACACTTTTTCATACATTGCCCAAGAATAAA
GAATCGTTTGTGTTATGTTTCAACGTGTTTATTTTTCAATTGCAGAAAATTTC
GAATCATTTTTCATTCAGTAGTATAGCCCCACCACCACATAGCTTATACAGAT
CACCGTACCTTAATCAAACTCACAGAACCCTAGTATTCAACCTGCCACCTCC
CTCCCAACACACAGAGTACACAGTCCTTTCTCCCCGGCTGGCCTTAAAAAG
CATCATATCATGGGTAACAGACATATTCTTAGGTGTTATATTCCACACGGTTT
CCTGTCGAGCCAAACGCTCATCAGTGATATTAATAAACTCCCCGGGCAGCT
CACTTAAGTTCATGTCGCTGTCCAGCTGCTGAGCCACAGGCTGCTGTCCAA
CTTGCGGTTGCTTAACGGGCGGCGAAGGAGAAGTCCACGCCTACATGGGG
GTAGAGTCATAATCGTGCATCAGGATAGGGCGGTGGTGCTGCAGCAGCGC
GCGAATAAACTGCTGCCGCCGCCGCTCCGTCCTGCAGGAATACAACATGGC
AGTGGTCTCCTCAGCGATGATTCGCACCGCCCGCAGCATAAGGCGCCTTGT
CCTCCGGGCACAGCAGCGCACCCTGATCTCACTTAAATCAGCACAGTAACT
GCAGCACAGCACCACAATATTGTTCAAAATCCCACAGTGCAAGGCGCTGTA
TCCAAAGCTCATGGCGGGGACCACAGAACCCACGTGGCCATCATACCACAA
GCGCAGGTAGATTAAGTGGCGACCCCTCATAAACACGCTGGACATAAACAT
TACCTCTTTTGGCATGTTGTAATTCACCACCTCCCGGTACCATATAAACCTCT
GATTAAACATGGCGCCATCCACCACCATCCTAAACCAGCTGGCCAAAACCT
GCCCGCCGGCTATACACTGCAGGGAACCGGGACTGGAACAATGACAGTGG
AGAGCCCAGGACTCGTAACCATGGATCATCATGCTCGTCATGATATCAATGT
TGGCACAACACAGGCACACGTGCATACACTTCCTCAGGATTACAAGCTCCT
CCCGCGTTAGAACCATATCCCAGGGAACAACCCATTCCTGAATCAGCGTAA
ATCCCACACTGCAGGGAAGACCTCGCACGTAACTCACGTTGTGCATTGTCA
AAGTGTTACATTCGGGCAGCAGCGGATGATCCTCCAGTATGGTAGCGCGGG
TTTCTGTCTCAAAAGGAGGTAGACGATCCCTACTGTACGGAGTGCGCCGAG
ACAACCGAGATCGTGTTGGTCGTAGTGTCATGCCAAATGGAACGCCGGACG
TAGTCATATTTCCTGAAGCAAAACCAGGTGCGGGCGTGACAAACAGATCTG
CGTCTCCGGTCTCGCCGCTTAGATCGCTCTGTGTAGTAGTTGTAGTATATCC
ACTCTCTCAAAGCATCCAGGCGCCCCCTGGCTTCGGGTTCTATGTAAACTC
CTTCATGCGCCGCTGCCCTGATAACATCCACCACCGCAGAATAAGCCACAC
CCAGCCAACCTACACATTCGTTCTGCGAGTCACACACGGGAGGAGCGGGA
AGAGCTGGAAGAACCATGTTTTTTTTTTTATTCCAAAAGATTATCCAAAACCT
CAAAATGAAGATCTATTAAGTGAACGCGCTCCCCTCCGGTGGCGTGGTCAA
ACTCTACAGCCAAAGAACAGATAATGGCATTTGTAAGATGTTGCACAATGGC
TTCCAAAAGGCAAACGGCCCTCACGTCCAAGTGGACGTAAAGGCTAAACCC
TTCAGGGTGAATCTCCTCTATAAACATTCCAGCACCTTCAACCATGCCCAAA
TAATTCTCATCTCGCCACCTTCTCAATATATCTCTAAGCAAATCCCGAATATT
AAGTCCGGCCATTGTAAAAATCTGCTCCAGAGCGCCCTCCACCTTCAGCCT
CAAGCAGCGAATCATGATTGCAAAAATTCAGGTTCCTCACAGACCTGTATAA
GATTCAAAAGCGGAACATTAACAAAAATACCGCGATCCCGTAGGTCCCTTCG
CAGGGCCAGCTGAACATAATCGTGCAGGTCTGCACGGACCAGCGCGGCCA
CTTCCCCGCCAGGAACCTTGACAAAAGAACCCACACTGATTATGACACGCA
TACTCGGAGCTATGCTAACCAGCGTAGCCCCGATGTAAGCTTTGTTGCATG
GGCGGCGATATAAAATGCAAGGTGCTGCTCAAAAAATCAGGCAAAGCCTCG
CGCAAAAAAGAAAGCACATCGTAGTCATGCTCATGCAGATAAAGGCAGGTA
AGCTCCGGAACCACCACAGAAAAAGACACCATTTTTCTCTCAAACATGTCTG
CGGGTTTCTGCATAAACACAAAATAAAATAACAAAAAAACATTTAAACATTAG
AAGCCTGTCTTACAACAGGAAAAACAACCCTTATAAGCATAAGACGGACTAC
GGCCATGCCGGCGTGACCGTAAAAAAACTGGTCACCGTGATTAAAAAGCAC
CACCGACAGCTCCTCGGTCATGTCCGGAGTCATAATGTAAGACTCGGTAAA
CACATCAGGTTGATTCACATCGGTCAGTGCTAAAAAGCGACCGAAATAGCC
CGGGGGAATACATACCCGCAGGCGTAGAGACAACATTACAGCCCCCATAG
GAGGTATAACAAAATTAATAGGAGAGAAAAACACATAAACACCTGAAAAACC
CTCCTGCCTAGGCAAAATAGCACCCTCCCGCTCCAGAACAACATACAGCGC
TTCCACAGCGGCAGCCATAACAGTCAGCCTTACCAGTAAAAAAGAAAACCTA
TTAAAAAAACACCACTCGACACGGCACCAGCTCAATCAGTCACAGTGTAAAA
AAGGGCCAAGTGCAGAGCGAGTATATATAGGACTAAAAAATGACGTAACGG
TTAAAGTCCACAAAAAACACCCAGAAAACCGCACGCGAACCTACGCCCAGA
AACGAAAGCCAAAAAACCCACAACTTCCTCAAATCGTCACTTCCGTTTTCCC
ACGTTACGTCACTTCCCATTTTAAGAAAACTACAATTCCCAACACATACAAGT
TACTCCGCCCTAAAACCTACGTCACCCGCCCCGTTCCCACGCCCCGCGCCA
CGTCACAAACTCCACCCCCTCATTATCATATTGGCTTCAATCCAAAATAAGG
TATATTATTGATGATGTTAAT
AdCh63 genome.
SEQ ID NO: 8
Underlined region may be replaced with promoter + antigen + polyA sequence
AAACCATCATCAATAATATACCTCAAACTTTTGGTGCGCGTTAATATGCAAAT
GAGCTGTTTGAATTTGGGGAGGGAGGAAGGTGATTGGCTGCGGGAGCGGC
GACCGTTAGGGGCGGGGCGGGTGACGTTTTGATGACGTGGCTATGAGGCG
GAGCCGGTTTGCAAGTTCTCGTGGGAAAAGTGACGTCAAACGAGGTGTGGT
TTGAACACGGAAATACTCAATTTTCCCGCGCTCTCTGACAGGAAATGAGGTG
TTTCTGGGCGGATGCAAGTGAAAACGGGCCATTTTCGCGCGAAAACTGAAT
GAGGAAGTGAAAATCTGAGTAATTTCGCGTTTATGGCAGGGAGGAGTATTT
GCCGAGGGCCGAGTAGACTTTGACCGATTACGTGGGGGTTTCGATTACCGT
ATTTTTCACCTAAATTTCCGCGTACGGTGTCAAAGTCCGGTGTTTTTACgcgat
cgcTAGCGACATCGATCACAAGTTTGTACAAAAAAGCTGAACGAGAAACGTAA
AATGATATAAATATCAATATATTAAATTAGATTTTGCATAAAAAACAGACTACA
TAATACTGTAAAACACAACATATCCAGTCACTATGGCGGCCGCATTAGGCAC
CCCAGGCTTTACACTTTATGCTTCCGGCTCGTATAATGTGTGGATTTTGAGT
TAGGATCCGGCGAGATTTTCAGGAGCTAAGGAAGCTAAAATGGAGAAAAAA
ATCACTGGATATACCACCGTTGATATATCCCAATGGCATCGTAAAGAACATT
TTGAGGCATTTCAGTCAGTTGCTCAATGTACCTATAACCAGACCGTTCAGCT
GGATATTACGGCCTTTTTAAAGACCGTAAAGAAAAATAAGCACAAGTTTTATC
CGGCCTTTATTCACATTCTTGCCCGCCTGATGAATGCTCATCCGGAATTCCG
TATGGCAATGAAAGACGGTGAGCTGGTGATATGGGATAGTGTTCACCCTTG
TTACACCGTTTTCCATGAGCAAACTGAAACGTTTTCATCGCTCTGGAGTGAA
TACCACGACGATTTCCGGCAGTTTCTACACATATATTCGCAAGATGTGGCGT
GTTACGGTGAAAACCTGGCCTATTTCCCTAAAGGGTTTATTGAGAATATGTT
TTTCGTCTCAGCCAATCCCTGGGTGAGTTTCACCAGTTTTGATTTAAACGTG
GCCAATATGGACAACTTCTTCGCCCCCGTTTTCACCATGGGCAAATATTATA
CGCAAGGCGACAAGGTGCTGATGCCGCTGGCGATTCAGGTTCATCATGCC
GTCTGTGATGGCTTCCATGTCGGCAGAATGCTTAATGAATTACAACAGTACT
GCGATGAGTGGCAGGGCGGGGCGTAAACGCGTGGATCCGGCTTACTAAAA
GCCAGATAACAGTATGCGTATTTGCGCGCTGATTTTTGCGGTATAAGAATAT
ATACTGATATGTATACCCGAAGTATGTCAAAAAGAGGTGTGCTATGAAGCAG
CGTATTACAGTGACAGTTGACAGCGACAGCTATCAGTTGCTCAAGGCATATA
TGATGTCAATATCTCCGGTCTGGTAAGCACAACCATGCAGAATGAAGCCCG
TCGTCTGCGTGCCGAACGCTGGAAAGCGGAAAATCAGGAAGGGATGGCTG
AGGTCGCCCGGTTTATTGAAATGAACGGCTCTTTTGCTGACGAGAACAGGG
ACTGGTGAAATGCAGTTTAAGGTTTACACCTATAAAAGAGAGAGCCGTTATC
GTCTGTTTGTGGATGTACAGAGTGATATTATTGACACGCCCGGGCGACGGA
TGGTGATCCCCCTGGCCAGTGCACGTCTGCTGTCAGATAAAGTCTCCCGTG
AACTTTACCCGGTGGTGCATATCGGGGATGAAAGCTGGCGCATGATGACCA
CCGATATGGCCAGTGTGCCGGTCTCCGTTATCGGGGAAGAAGTGGCTGATC
TCAGCCACCGCGAAAATGACATCAAAAACGCCATTAACCTGATGTTCTGGG
GAATATAAATGTCAGGCTCCGTTATACACAGCCAGTCTGCAGGTCGACCATA
GTGACTGGATATGTTGTGTTTTACAGTATTATGTAGTCTGTTTTTTATGCAAA
ATCTAATTTAATATATTGATATTTATATCATTTTACGTTTCTCGTTCAGCTTTCT
TGTACAAAGTGGTGATCGATTCGACAGATCgcgatCGCGTGAGTAGTGTTTGG
GGCTGGGTGTGAGCCTGCATGAGGGGCAGAATGACTAAAATCTGTGGTTTT
CTGTGTGTTGCAGCAGCATGAGCGGAAGCGCCTCCTTTGAGGGAGGGGTA
TTCAGCCCTTATCTGACGGGGCGTCTCCCCTCCTGGGCGGGAGTGTGTCA
GAATGTTATGGGATCCACGGTGGACGGCCGGCCCGTGCAGCCCGCGAACT
CTTCAACCCTGACCTACGCGACCCTGAGCTCCTCGTCCGTGGACGCAGCTG
CCGCCGCAGCTGCTGCTTCCGCCGCCAGCGCCGTGCGCGGAATGGCCCT
GGGCGCCGGCTACTACAGCTCTCTGGTGGCCAACTCGAGTTCCACCAATAA
TCCCGCCAGCCTGAACGAGGAGAAGCTGTTGCTGCTGATGGCCCAGCTCG
AGGCCCTGACCCAGCGCCTGGGCGAGCTGACCCAGCAGGTGGCTCAGCTG
CAGGCGGAGACGCGGGCCGCGGTTGCCACGGTGAAAACCAAATAAAAAAT
GAATCAATAAATAAACGGAGACGGTTGTTGATTTTAACACAGAGTCTTGAAT
CTTTATTTGATTTTTCGCGCGCGGTAGGCCCTGGACCACCGGTCTCGATCAT
TGAGCACCCGGTGGATCTTTTCCAGGACCCGGTAGAGGTGGGCTTGGATGT
TGAGGTACATGGGCATGAGCCCGTCCCGGGGGTGGAGGTAGCTCCATTGC
AGGGCCTCGTGCTCGGGGGTGGTGTTGTAAATCACCCAGTCATAGCAGGG
GCGCAGGGCGTGGTGCTGCACGATGTCTTTGAGGAGGAGACTGATGGCCA
CGGGCAGCCCCTTGGTGTAGGTGTTGACGAACCTATTGAGCTGGGAGGGA
TGCATGCGGGGGGAGATGAGATGCATCTTGGCCTGGATCTTGAGATTGGC
GATGTTCCCGCCCAGATCCCGCCGGGGGTTCATGTTGTGCAGGACCACCA
GCACGGTGTATCCGGTGCACTTGGGGAATTTGTCATGCAACTTGGAAGGGA
AGGCGTGAAAGAATTTGGAGACGCCCTTGTGACCGCCCAGGTTTTCCATGC
ACTCATCCATGATGATGGCGATGGGCCCGTGGGCGGCGGCCTGGGCAAAG
ACGTTTCGGGGGTCGGACACATCGTAGTTGTGGTCCTGGGTGAGCTCGTCA
TAGGCCATTTTAATGAATTTGGGGCGGAGGGTACCCGACTGGGGGACAAAG
GTGCCCTCGATCCCGGGGGCGTAGTTCCCCTCGCAGATCTGCATCTCCCA
GGCCTTGAGCTCGGAGGGGGGGATCATGTCCACCTGCGGGGCGATGAAAA
AAACGGTTTCCGGGGCGGGGGAGATGAGCTGCGCCGAAAGCAGGTTCCGG
AGCAGCTGGGACTTGCCGCAGCCGGTGGGGCCGTAGATGACCCCGATGAC
CGGCTGCAGGTGGTAGTTGAGGGAGAGACAGCTGCCGTCCTCGCGGAGGA
GGGGGGCCACCTCGTTCATCATCTCGCGCACATGCATGTTCTCGCGCACGA
GTTCCGCCAGGAGGCGCTCGCCCCCCAGCGAGAGGAGCTCTTGCAGCGAG
GCGAAGTTTTTCAGCGGCTTGAGCCCGTCGGCCATGGGCATTTTGGAGAGG
GTCTGTTGCAAGAGTTCCAGACGGTCCCAGAGCTCGGTGATGTGCTCTAGG
GCATCTCGATCCAGCAGACCTCCTCGTTTCGCGGGTTGGGGCGACTGCGG
GAGTAGGGCACCAGGCGATGGGCGTCCAGCGAGGCCAGGGTCCGGTCCT
TCCAGGGTCGCAGGGTCCGCGTCAGCGTGGTCTCCGTCACGGTGAAGGGG
TGCGCGCCGGGCTGGGCGCTTGCGAGGGTGCGCTTCAGGCTCATCCGGCT
GGTCGAGAACCGCTCCCGGTCGGCGCCCTGCGCGTCGGCCAGGTAGCAAT
TGAGCATGAGTTCGTAGTTGAGCGCCTCGGCCGCGTGGCCCTTGGCGCGG
AGCTTACCTTTGGAAGTGTGTCCGCAGACGGGACAGAGGAGGGACTTGAG
GGCGTAGAGCTTGGGGGCGAGGAAGACGGACTCGGGGGCGTAGGCGTCC
GCGCCGCAGCTGGCGCAGACGGTCTCGCACTCCACGAGCCAGGTGAGGTC
GGGGCGGTCGGGGTCAAAAACGAGGTTTCCTCCGTGCTTTTTGATGCGTTT
CTTACCTCTGGTCTCCATGAGCTCGTGTCCCCGCTGGGTGACAAAGAGGCT
GTCCGTGTCCCCGTAGACCGACTTTATGGGCCGGTCCTCGAGCGGGGTGC
CGCGGTCCTCGTCGTAGAGGAACCCCGCCCACTCCGAGACGAAGGCCCGG
GTCCAGGCCAGCACGAAGGAGGCCACGTGGGAGGGGTAGCGGTCGTTGT
CCACCAGCGGGTCCACCTTCTCCAGGGTATGCAAGCACATGTCCCCCTCGT
CCACATCCAGGAAGGTGATTGGCTTGTAAGTGTAGGCCACGTGACCGGGG
GTCCCGGCCGGGGGGGTATAAAAGGGGGCGGGCCCCTGCTCGTCCTCACT
GTCTTCCGGATCGCTGTCCAGGAGCGCCAGCTGTTGGGGTAGGTATTCCCT
CTCGAAGGCGGGCATGACCTCGGCACTCAGGTTGTCAGTTTCTAGAAACGA
GGAGGATTTGATATTGACGGTGCCGTTGGAGACGCCTTTCATGAGCCCCTC
GTCCATCTGGTCAGAAAAGACGATCTTTTTGTTGTCGAGCTTGGTGGCGAA
GGAGCCGTAGAGGGCGTTGGAGAGCAGCTTGGCGATGGAGCGCATGGTCT
GGTTCTTTTCCTTGTCGGCGCGCTCCTTGGCGGCGATGTTGAGCTGCACGT
ACTCGCGCGCCACGCACTTCCATTCGGGGAAGACGGTGGTGAGCTCGTCG
GGCACGATTCTGACCCGCCAGCCGCGGTTGTGCAGGGTGATGAGGTCCAC
GCTGGTGGCCACCTCGCCGCGCAGGGGCTCGTTGGTCCAGCAGAGGCGC
CCGCCCTTGCGCGAGCAGAAGGGGGGCAGCGGGTCCAGCATGAGCTCGT
CGGGGGGGTCGGCGTCCACGGTGAAGATGCCGGGCAGGAGCTCGGGGTC
GAAGTAGCTGATGCAGGTGCCCAGATCGTCCAGACTTGCTTGCCAGTCGCG
CACGGCCAGCGCGCGCTCGTAGGGGCTGAGGGGCGTGCCCCAGGGCATG
GGGTGCGTGAGCGCGGAGGCGTACATGCCGCAGATGTCGTAGACGTAGAG
GGGCTCCTGGAGGACGCCGATGTAGGTGGGGTAGCAGCGCCCCCCGCGG
ATGCTGGCGCGCACGTAGTCGTACAGCTCGTGCGAGGGCGCGAGGAGCCC
CGTGCCGAGATTGGAGCGCTGCGGCTTTTCGGCGCGGTAGACGATCTGGC
GGAAGATGGCGTGGGAGTTGGAGGAGATGGTGGGCCTCTGGAAGATGTTG
AAGTGGGCATGGGGCAGTCCGACCGAGTCCCTGATGAAGTGGGCGTAGGA
GTCCTGCAGCTTGGCGACGAGCTCGGCGGTGACGAGGACGTCCAGGGCG
CAGTAGTCGAGGGTCTCTTGGATGATGTCGTACTTGAGCTGGCCCTTCTGC
TTCCACAGCTCGCGGTTGAGAAGGAACTCTTCGCGGTCCTTCCAGTACTCT
TCGAGGGGGAACCCGTCCTGATCGGCACGGTAAGAGCCCACCATGTAGAA
CTGGTTGACGGCCTTGTAGGCGCAGCAGCCCTTCTCCACGGGGAGGGCGT
AAGCTTGCGCGGCCTTGCGCAGGGAGGTGTGGGTGAGGGCGAAGGTGTC
GCGCACCATGACTTTGAGGAACTGGTGCTTGAAGTCGAGGTCGTCGCAGCC
GCCCTGCTCCCAGAGCTGGAAGTCCGTGCGCTTCTTGTAGGCGGGGTTGG
GCAAAGCGAAAGTAACATCGTTGAAGAGGATCTTGCCCGCGCGGGGCATG
AAGTTGCGAGTGATGCGGAAAGGCTGGGGCACCTCGGCCCGGTTGTTGAT
GACCTGGGCGGCGAGGACGATCTCGTCGAAGCCGTTGATGTTGTGCCCGA
CGATGTAGAGTTCCACGAATCGCGGGCGGCCCTTGACGTGGGGCAGCTTC
TTGAGCTCGTCGTAGGTGAGCTCGGCGGGGTCGCTGAGCCCGTGCTGCTC
GAGGGCCCAGTCGGCGACGTGGGGGTTGGCGCTGAGGAAGGAAGTCCAG
AGATCCACGGCCAGGGCGGTCTGCAAGCGGTCCCGGTACTGACGGAACTG
CTGGCCCACGGCCATTTTTTCGGGGGTGACGCAGTAGAAGGTGCGGGGGT
CGCCGTGCCAGCGGTCCCACTTGAGCTGGAGGGCGAGGTCGTGGGCGAG
CTCGACGAGCGGCGGGTCCCCGGAGAGTTTCATGACCAGCATGAAGGGGA
CGAGCTGCTTGCCGAAGGACCCCATCCAGGTGTAGGTTTCCACATCGTAGG
TGAGGAAGAGCCTTTCGGTGCGAGGATGCGAGCCGATGGGGAAGAACTGG
ATCTCCTGCCACCAGTTGGAGGAATGGCTGTTGATGTGATGGAAGTAGAAA
TGCCGACGGCGCGCCGAGCACTCGTGCTTGTGTTTATACAAGCGTCCGCA
GTGCTCGCAACGCTGCACGGGATGCACGTGCTGCACGAGCTGTACCTGGG
TTCCTTTGACGAGGAATTTCAGTGGGCAGTGGAGCGCTGGCGGCTGCATCT
GGTGCTGTACTACGTCCTGGCCATCGGCGTGGCCATCGTCTGCCTCGATG
GTGGTCATGCTGACGAGCCCGCGCGGGAGGCAGGTCCAGACCTCGGCTC
GGACGGGTCGGAGAGCGAGGACGAGGGCGCGCAGGCCGGAGCTGTCCAG
GGTCCTGAGACGCTGCGGAGTCAGGTCAGTGGGCAGCGGCGGCGCGCGG
TTGACTTGCAGGAGCTTTTCCAGGGCGCGCGGGAGGTCCAGATGGTACTTG
ATCTCCACGGCGCCGTTGGTGGCGACGTCCACGGCTTGCAGGGTCCCGTG
CCCCTGGGGCGCCACCACCGTGCCCCGTTTCTTCTTGGGCGGCGGCGGCT
CCATGCTTAGAAGCGGCGGCGAGGACGCGCGCCGGGCGGCAGGGGCGGC
TCGGGGCCCGGAGGCAGGGGCGGCAGGGGCACGTCGGCGCCGCGCGCG
GGCAGGTTCTGGTACTGCGCCCGGAGAAGACTGGCGTGAGCGACGACGCG
ACGGTTGACGTCCTGGATCTGACGCCTCTGGGTGAAGGCCACGGGACCCG
TGAGTTTGAACCTGAAAGAGAGTTCGACAGAATCAATCTCGGTATCGTTGAC
GGCGGCCTGCCGCAGGATCTCTTGCACGTCGCCCGAGTTGTCCTGGTAGG
CGATCTCGGTCATGAACTGCTCGATCTCCTCCTCCTGAAGGTCTCCGCGGC
CGGCGCGCTCGACGGTGGCCGCGAGGTCGTTGGAGATGCGGCCCATGAG
CTGCGAGAAGGCGTTCATGCCGGCCTCGTTCCAGACGCGGCTGTAGACCA
CGGCTCCGTCGGGGTCGCGCGCGCGCATGACCACCTGGGCGAGGTTGAG
CTCGACGTGGCGCGTGAAGACCGCGTAGTTGCAGAGGCGCTGGTAGAGGT
AGTTGAGCGTGGTGGCGATGTGCTCGGTGACGAAGAAGTACATGATCCAGC
GGCGGAGCGGCATCTCGCTGACGTCGCCCAGGGCTTCCAAGCGCTCCATG
GCCTCGTAGAAGTCCACGGCGAAGTTGAAAAACTGGGAGTTGCGCGCCGA
GACGGTCAACTCCTCCTCCAGAAGACGGATGAGCTCGGCGATGGTGGCGC
GCACCTCGCGCTCGAAGGCCCCGGGGGGCTCCTCTTCCATTTCCTCCTCTT
CCTCCTCCACTAACATCTCTTCTACTTCCTCCTCAGGAGGCGGCGGCGGGG
GAGGGGCCCTGCGTCGCCGGCGGCGCACGGGCAGACGGTCGATGAAGCG
CTCGATGGTCTCCCCGCGCCGGCGACGCATGGTCTCGGTGACGGCGCGCC
CGTCCTCGCGGGGCCGCAGCGTGAAGACGCCGCCGCGCATCTCCAGGTG
GCCGCCGGGGGGGTCTCCGTTGGGCAGGGAGAGGGCGCTGACGATGCAT
CTTATCAATTGACCCGTAGGGACTCCGCGCAAGGACCTGAGCGTCTCGAGA
TCCACGGGATCCGAAAACCGCTGAACGAAGGCTTCGAGCCAGTCGCAGTC
GCAAGGTAGGCTGAGCCCGGTTTCTTGTTCTTCGGGTATTTGGTCGGGAGG
CGGGCGGGCGATGCTGCTGGTGATGAAGTTGAAGTAGGCGGTCCTGAGAC
GGCGGATGGTGGCGAGGAGCACCAGGTCCTTGGGCCCGGCTTGCTGGAT
GCGCAGACGGTCGGCCATGCCCCAGGCGTGGTCCTGACACCTGGCGAGGT
CCTTGTAGTAGTCCTGCATGAGCCGCTCCACGGGCACCTCCTCCTCGCCCG
CGCGGCCGTGCATGCGCGTGAGCCCGAACCCGCGCTGCGGCTGGACGAG
CGCCAGGTCGGCGACGACGCGCTCGGCGAGGATGGCCTGCTGGATCTGG
GTGAGGGTGGTCTGGAAGTCGTCGAAGTCGACGAAGCGGTGGTAGGCTCC
GGTGTTGATGGTGTAGGAGCAGTTGGCCATGACGGACCAGTTGACGGTCT
GGTGGCCGGGGCGCACGAGCTCGTGGTACTTGAGGCGCGAGTAGGCGCG
CGTGTCGAAGATGTAGTCGTTGCAGGTGCGCACGAGGTACTGGTATCCGAC
GAGGAAGTGCGGCGGCGGCTGGCGGTAGAGCGGCCATCGCTCGGTGGCG
GGGGCGCCGGGCGCGAGGTCCTCGAGCATGAGGCGGTGGTAGCCGTAGA
TGTACCTGGACATCCAGGTGATGCCGGCGGCGGTGGTGGAGGCGCGCGG
GAACTCGCGGACGCGGTTCCAGATGTTGCGCAGCGGCAGGAAGTAGTTCA
TGGTGGCCGCGGTCTGGCCCGTGAGGCGCGCGCAGTCGTGGATGCTCTAG
ACATACGGGCAAAAACGAAAGCGGTCAGCGGCTCGACTCCGTGGCCTGGA
GGCTAAGCGAACGGGTTGGGCTGCGCGTGTACCCCGGTTCGAATCTCGAA
TCAGGCTGGAGCCGCAGCTAACGTGGTACTGGCACTCCCGTCTCGACCCA
AGCCTGCTAACGAAACCTCCAGGATACGGAGGCGGGTCGTTTTTTGGCCTT
GGTCGCTGGTCATGAAAAACTAGTAAGCGCGGAAAGCGGCCGCCCGCGAT
GGCTCGCTGCCGTAGTCTGGAGAAAGAATCGCCAGGGTTGCGTTGCGGTG
TGCCCCGGTTCGAGCCTCAGCGCTCGGCGCCGGCCGGATTCCGCGGCTAA
CGTGGGCGTGGCTGCCCCGTCGTTTCCAAGACCCCTTAGCCAGCCGACTT
CTCCAGTTACGGAGCGAGCCCCTCTTTTTTTCTTGTGTTTTTGCCAGATGCA
TCCCGTACTGCGGCAGATGCGCCCCCACCCTCCACCACAACCGCCCCTAC
CGCAGCAGCAGCAACAGCCGGCGCTTCTGCCCCCGCCCCAGCAGCAGCAG
CCAGCCACTACCGCGGCGGCCGCCGTGAGCGGAGCCGGCGTTCAGTATGA
CCTGGCCTTGGAAGAGGGCGAGGGGCTGGCGCGGCTGGGGGCGTCGTCG
CCGGAGCGGCACCCGCGCGTGCAGATGAAAAGGGACGCTCGCGAGGCCT
ACGTGCCCAAGCAGAACCTGTTCAGAGACAGGAGCGGCGAGGAGCCCGAG
GAGATGCGCGCCTCCCGCTTCCACGCGGGGCGGGAGCTGCGGCGCGGCC
TGGACCGAAAGCGGGTGCTGAGGGACGAGGATTTCGAGGCGGACGAGCTG
ACGGGGATCAGCCCCGCGCGCGCGCACGTGGCCGCGGCCAACCTGGTCA
CGGCGTACGAGCAGACCGTGAAGGAGGAGAGCAACTTCCAAAAATCCTTCA
ACAACCACGTGCGCACGCTGATCGCGCGCGAGGAGGTGACCCTGGGCCTG
ATGCACCTGTGGGACCTGCTGGAGGCCATCGTGCAGAACCCCACGAGCAA
GCCGCTGACGGCGCAGCTGTTTCTGGTGGTGCAGCACAGTCGGGACAACG
AGACGTTCAGGGAGGCGCTGCTGAATATCACCGAGCCCGAGGGCCGCTGG
CTCCTGGACCTGGTGAACATTCTGCAGAGCATCGTGGTGCAGGAGCGCGG
GCTGCCGCTGTCCGAGAAGCTGGCGGCCATCAACTTCTCGGTGCTGAGCC
TGGGCAAGTACTACGCTAGGAAGATCTACAAGACCCCGTACGTGCCCATAG
ACAAGGAGGTGAAGATCGATGGGTTTTACATGCGCATGACCCTGAAAGTGC
TGACCCTGAGCGACGATCTGGGGGTGTACCGCAACGACAGGATGCACCGC
GCGGTGAGCGCCAGCCGCCGGCGCGAGCTGAGCGACCAGGAGCTGATGC
ACAGCCTGCAGCGGGCCCTGACCGGGGCCGGGACCGAGGGGGAGAGCTA
CTTTGACATGGGCGCGGACCTGCGCTGGCAGCCCAGCCGCCGGGCCTTGG
AAGCTGCCGGCGGCGTGCCCTACGTGGAGGAGGTGGACGATGAGGAGGA
GGAGGGCGAGTACCTGGAAGACTGATGGCGCGACCGTATTTTTGCTAGATG
CAGCAACAGCCACCGCCGCCGCCTCCTGATCCCGCGATGCGGGCGGCGCT
GCAGAGCCAGCCGTCCGGCATTAACTCCTCGGACGATTGGACCCAGGCCA
TGCAACGCATCATGGCGCTGACGACCCGCAATCCCGAAGCCTTTAGACAGC
AGCCTCAGGCCAACCGGCTCTCGGCCATCCTGGAGGCCGTGGTGCCCTCG
CGCTCGAACCCCACGCACGAGAAGGTGCTGGCCATCGTGAACGCGCTGGT
GGAGAACAAGGCCATCCGCGGCGACGAGGCCGGGCTGGTGTACAACGCG
CTGCTGGAGCGCGTGGCCCGCTACAACAGCACCAACGTGCAGACGAACCT
GGACCGCATGGTGACCGACGTGCGCGAGGCGGTGTCGCAGCGCGAGCGG
TTCCACCGCGAGTCGAACCTGGGCTCCATGGTGGCGCTGAACGCCTTCCT
GAGCACGCAGCCCGCCAACGTGCCCCGGGGCCAGGAGGACTACACCAACT
TCATCAGCGCGCTGCGGCTGATGGTGGCCGAGGTGCCCCAGAGCGAGGTG
TACCAGTCGGGGCCGGACTACTTCTTCCAGACCAGTCGCCAGGGCTTGCA
GACCGTGAACCTGAGCCAGGCTTTCAAGAACTTGCAGGGACTGTGGGGCG
TGCAGGCCCCGGTCGGGGACCGCGCGACGGTGTCGAGCCTGCTGACGCC
GAACTCGCGCCTGCTGCTGCTGCTGGTGGCGCCCTTCACGGACAGCGGCA
GCGTGAGCCGCGACTCGTACCTGGGCTACCTGCTTAACCTGTACCGCGAG
GCCATCGGGCAGGCGCACGTGGACGAGCAGACCTACCAGGAGATCACCCA
CGTGAGCCGCGCGCTGGGCCAGGAGGACCCGGGCAACCTGGAGGCCACC
CTGAACTTCCTGCTGACCAACCGGTCGCAGAAGATCCCGCCCCAGTACGCG
CTGAGCACCGAGGAGGAGCGCATCCTGCGCTACGTGCAGCAGAGCGTGGG
GCTGTTCTTGATGCAGGAGGGGGCCACGCCCAGCGCCGCGCTCGACATGA
CCGCGCGCAACATGGAGCCCAGCATGTACGCCCGCAACCGCCCGTTCATC
AATAAGCTGATGGACTACTTGCATCGGGCGGCCGCCATGAACTCGGACTAC
TTTACCAACGCCATCTTGAACCCGCACTGGCTCCCGCCGCCCGGGTTCTAC
ACGGGCGAGTACGACATGCCCGACCCCAACGACGGGTTCCTGTGGGACGA
CGTGGACAGCAGCGTGTTCTCGCCGCGGCCCACCACCACCACCGTGTGGA
AGAAAGAGGGCGGGGACCGGCGGCCGTCCTCGGCGCTGTCCGGTCGCGC
GGGTGCTGCCGCGGCGGTGCCCGAGGCTGCCAGCCCCTTCCCGAGCCTG
CCCTTTTCGCTGAACAGCGTGCGCAGCAGCGAGCTGGGTCGGCTGACGCG
GCCGCGCCTGCTGGGCGAGGAGGAGTACCTGAACGACTCCTTGTTGAAGC
CCGAGCGCGAGAAGAACTTCCCCAATAACGGGATAGAGAGCCTGGTGGAC
AAGATGAGCCGCTGGAAGACGTACGCGCACGAGCACAGGGACGAGCCCCG
AGCTAGCAGCGCAGGCACCCGTAGACGCCAGCGGCACGACAGGCAGCGG
GGACTGGTGTGGGACGATGAGGATTCCGCCGACGACAGCAGCGTGTTGGA
CTTGGGTGGGAGTGGTGGTGGTAACCCGTTCGCTCACCTGCGCCCCCGTA
TCGGGCGCCTGATGTAAGAATCTGAAAAAATAAAAGACGGTACTCACCAAG
GCCATGGCGACCAGCGTGCGTTCTTCTCTGTTGTTTGTAGTAGTATGATGAG
GCGCGTGTACCCGGAGGGTCCTCCTCCCTCGTACGAGAGCGTGATGCAGC
AGGCGGTGGCGGCGGCGATGCAGCCCCCGCTGGAGGCGCCTTACGTGCC
CCCGCGGTACCTGGCGCCTACGGAGGGGCGGAACAGCATTCGTTACTCGG
AGCTGGCACCCTTGTACGATACCACCCGGTTGTACCTGGTGGACAACAAGT
CGGCGGACATCGCCTCGCTGAACTACCAGAACGACCACAGCAACTTCCTGA
CCACCGTGGTGCAGAACAACGATTTCACCCCCACGGAGGCCAGCACCCAG
ACCATCAACTTTGACGAGCGCTCGCGGTGGGGCGGCCAGCTGAAAACCAT
CATGCACACCAACATGCCCAACGTGAACGAGTTCATGTACAGCAACAAGTT
CAAGGCGCGGGTGATGGTCTCGCGCAAGACCCCCAACGGGGTCACGGTAG
GGGATGATTATGATGGTAGTCAGGACGAGCTGACCTACGAGTGGGTGGAGT
TTGAGCTGCCCGAGGGCAACTTCTCGGTGACCATGACCATCGATCTGATGA
ACAACGCCATCATCGACAACTACTTGGCGGTGGGGCGGCAGAACGGGGTG
CTGGAGAGCGACATCGGCGTGAAGTTCGACACGCGCAACTTCCGGCTGGG
CTGGGACCCCGTGACCGAGCTGGTGATGCCGGGCGTGTACACCAACGAGG
CCTTCCACCCCGACATCGTCCTGCTGCCCGGCTGCGGCGTGGACTTCACC
GAGAGCCGCCTCAGCAACCTGCTGGGCATCCGCAAGCGGCAGCCCTTCCA
GGAGGGCTTCCAGATCCTGTACGAGGACCTGGAGGGGGGCAACATCCCCG
CGCTCTTGGATGTCGAAGCCTATGAAGAAAGTAAGGAAAAAGCAGAGGCTG
AGGCAACTACAGCCGTGGCTACCGCCGCGACTGTGGCAGATGCCACTGTC
ACCAGGGGCGATACATTCGCCACCCAGGCGGAGGAAGCAGCCGCCCTAGC
GGCGACCGATGATAGTGAAAGTAAGATAGTCATCAAGCCGGTGGAGAAGGA
CAGCAAGAACAGGAGCTACAACGTTCTACCGGATGGAAAGAACACCGCCTA
CCGCAGCTGGTACCTGGCCTACAACTACGGCGACCCCGAGAAGGGCGTGC
GCTCCTGGACGCTGCTCACCACCTCGGACGTCACCTGCGGCGTGGAGCAA
GTCTACTGGTCGCTGCCCGACATGATGCAAGACCCGGTCACCTTCCGCTCC
ACGCGACAAGTTAGCAACTACCCGGTGGTGGGCGCCGAGCTCCTGCCCGT
CTACTCCAAGAGCTTCTTCAACGAGCAGGCCGTCTACTCGCAGCAGCTGCG
TGCCTTCACCTCGCTCACGCACGTCTTCAACCGCTTCCCCGAGAACCAGAT
CCTCGTCCGCCCGCCCGCGCCCACCATTACCACCGTCAGTGAAAACGTTCC
TGCTCTCACAGATCACGGGACCCTGCCGCTGCGCAGCAGTATCCGGGGAG
TCCAGCGCGTGACCGTCACTGACGCCAGACGCCGCACCTGCCCCTACGTC
TACAAGGCCCTGGGCGTAGTCGCGCCGCGCGTCCTCTCGAGCCGCACCTT
CTAAAAAATGTCCATTCTCATCTCGCCCAGTAATAACACCGGTTGGGGCCTG
CGCGCGCCCAGCAAGATGTACGGAGGCGCTCGCCAACGCTCCACGCAACA
CCCCGTGCGCGTGCGCGGGCACTTCCGCGCTCCCTGGGGCGCCCTCAAG
GGCCGCGTGCGCTCGCGCACCACCGTCGACGACGTGATCGACCAGGTGGT
GGCCGACGCGCGCAACTACACGCCCGCCGCCGCGCCCGCCTCCACCGTG
GACGCCGTCATCGACAGCGTGGTGGCCGACGCGCGCCGGTACGCCCGCG
CCAAGAGCCGGCGGCGGCGCATCGCCCGGCGGCACCGGAGCACCCCCGC
CATGCGCGCGGCGCGAGCCTTGCTGCGCAGGGCCAGGCGCACGGGACGC
AGGGCCATGCTCAGGGCGGCCAGACGCGCGGCCTCCGGCAGCAGCAGCG
CCGGCAGGACCCGCAGACGCGCGGCCACGGCGGCGGCGGCGGCCATCG
CCAGCATGTCCCGCCCGCGGCGCGGCAACGTGTACTGGGTGCGCGACGC
CGCCACCGGTGTGCGCGTGCCCGTGCGCACCCGCCCCCCTCGCACTTGAA
GATGCTGACTTCGCGATGTTGATGTGTCCCAGCGGCGAGGAGGATGTCCAA
GCGCAAATACAAGGAAGAGATGCTCCAGGTCATCGCGCCTGAGATCTACGG
CCCCGCGGCGGCGGTGAAGGAGGAAAGAAAGCCCCGCAAACTGAAGCGG
GTCAAAAAGGACAAAAAGGAGGAGGAAGATGACGGACTGGTGGAGTTTGTG
CGCGAGTTCGCCCCCCGGCGGCGCGTGCAGTGGCGCGGGCGGAAAGTGA
AACCGGTGCTGCGGCCCGGCACCACGGTGGTCTTCACGCCCGGCGAGCGT
TCCGGCTCCGCCTCCAAGCGCTCCTACGACGAGGTGTACGGGGACGAGGA
CATCCTCGAGCAGGCGGTCGAGCGTCTGGGCGAGTTTGCTTACGGCAAGC
GCAGCCGCCCCGCGCCCTTGAAAGAGGAGGCGGTGTCCATCCCGCTGGAC
CACGGCAACCCCACGCCGAGCCTGAAGCCGGTGACCCTGCAGCAGGTGCT
GCCGAGCGCGGCGCCGCGCCGGGGCTTCAAGCGCGAGGGCGGCGAGGA
TCTGTACCCGACCATGCAGCTGATGGTGCCCAAGCGCCAGAAGCTGGAGG
ACGTGCTGGAGCACATGAAGGTGGACCCCGAGGTGCAGCCCGAGGTCAAG
GTGCGGCCCATCAAGCAGGTGGCCCCGGGCCTGGGCGTGCAGACCGTGG
ACATCAAGATCCCCACGGAGCCCATGGAAACGCAGACCGAGCCCGTGAAG
CCCAGCACCAGCACCATGGAGGTGCAGACGGATCCCTGGATGCCAGCGGC
TTCCACCACCACCACTCGCCGAAGACGCAAGTACGGCGCGGCCAGCCTGC
TGATGCCCAACTACGCGCTGCATCCTTCCATCATCCCCACGCCGGGCTACC
GCGGCACGCGCTTCTACCGCGGCTACACCAGCAGCCGCCGCCGCAAGACC
ACCACCCGCCGCCGTCGTCGCAGCCGCCGCAGCAGCACCGCGACTTCCGC
CTTGGTGCGGAGAGTGTATCGCAGCGGGCGCGAGCCTCTGACCCTGCCGC
GCGCGCGCTACCACCCGAGCATCGCCATTTAACTACCGCCTCCTACTTGCA
GATATGGCCCTCACATGCCGCCTCCGCGTCCCCATTACGGGCTACCGAGG
AAGAAAGCCGCGCCGTAGAAGGCTGACGGGGAACGGGCTGCGTCGCCATC
ACCACCGGCGGCGGCGCGCCATCAGCAAGCGGTTGGGGGGAGGCTTCCT
GCCCGCGCTGATCCCCATCATCGCCGCGGCGATCGGGGCGATCCCCGGCA
TAGCTTCCGTGGCGGTGCAGGCCTCTCAGCGCCACTGAGACACAAAAAAGC
ATGGATTTGTAATAAAAAAATGGACTGACGCTCCTGGTCCTGTGATGTGTGT
TTTTAGATGGAAGACATCAATTTTTCGTCCCTGGCACCGCGACACGGCACG
CGGCCGTTTATGGGCACCTGGAGCGACATCGGCAACAGCCAACTGAACGG
GGGCGCCTTCAATTGGAGCAGTCTCTGGAGCGGGCTTAAGAATTTCGGGTC
CACGCTCAAAACCTATGGCAACAAGGCGTGGAACAGCAGCACAGGGCAGG
CGCTGAGGGAAAAGCTGAAAGAGCAGAACTTCCAGCAGAAGGTGGTCGAT
GGCCTGGCCTCGGGCATCAACGGGGTGGTGGACCTGGCCAACCAGGCCG
TGCAGAAACAGATCAACAGCCGCCTGGACGCGGTCCCGCCCGCGGGGTCC
GTGGAGATGCCCCAGGTGGAGGAGGAGCTGCCTCCCCTGGACAAGCGCG
GCGACAAGCGACCGCGTCCCGACGCGGAGGAGACGCTGCTGACGCACAC
GGACGAGCCGCCCCCGTACGAGGAGGCGGTGAAACTGGGTCTGCCCACCA
CGCGGCCCGTGGCGCCTCTGGCCACCGGGGTGCTGAAACCCAGCAGCAG
CAGCCAGCCCGCGACCCTGGACTTGCCTCCGCCTGCTTCCCGCCCCTCCA
CAGTGGCTAAGCCCCTGCCGCCGGTGGCCGTCGCGTCGCGCGCCCCCCG
AGGCCGCCCCCAGGCGAACTGGCAGAGCACTCTGAACAGCATCGTGGGTC
TGGGAGTGCAGAGTGTGAAGCGCCGCCGCTGCTATTAAAAGACACTGTAGC
GCTTAACTTGCTTGTCTGTGTGTGTATATGTATGTCCGCCGACCAGAAGGAG
GAAGAGGCGCGTCGCCGAGTTGCAAGATGGCCACCCCATCGATGCTGCCC
CAGTGGGCGTACATGCACATCGCCGGACAGGACGCTTCGGAGTACCTGAG
TCCGGGTCTGGTGCAGTTCGCCCGCGCCACAGACACCTACTTCAGTCTGG
GGAACAAGTTTAGGAACCCCACGGTGGCGCCCACGCACGATGTGACCACC
GACCGCAGCCAGCGGCTGACGCTGCGCTTCGTGCCCGTGGACCGCGAGG
ACAACACCTACTCGTACAAAGTGCGCTACACGCTGGCCGTGGGCGACAACC
GCGTGCTGGACATGGCCAGCACCTACTTTGACATCCGCGGCGTGCTGGAT
CGGGGCCCCAGCTTCAAACCCTACTCCGGCACCGCCTACAACAGCCTAGCT
CCCAAGGGAGCGCCCAACACCTCACAGTGGAAGGATTCCGACAGCAAAAT
GCATACTTTTGGAGTTGCTGCCATGCCCGGTGTTGTTGGTAAAAAAATAGAA
GCCGATGGTCTGCCTATTGGAATAGATTCATCCTCTGGAACTGACACCATAA
TTTATGCTGATAAAACTTTCCAACCAGAGCCACAGGTTGGAAGTGACAGTTG
GGTCGACACCAATGGTGCAGAGGAAAAATATGGAGGTAGAGCTCTTAAGGA
CACTACAAACATGAAGCCCTGCTACGGTTCTTTTGCCAGGCCTACCAACAAA
GAAGGTGGACAGGCTAACATAAAAGATTCTGAAACTGCCAGCACTACTCCTA
ACTATGATATAGATTTGGCATTCTTTGACAGCAAAAATATTGCAGCTAACTAC
GATCCAGATATTGTAATGTACACAGAAAATGTTGAGTTGCAAACTCCAGATA
CTCATATTGTGTTTAAGCCAGGAACTTCAGATGAAAGTTCAGAAGCCAATTT
GGGCCAGCAGGCCATGCCCAACAGACCCAACTACATCGGGTTCAGAGACA
ACTTTATCGGGCTCATGTACTACAACAGCACTGGCAATATGGGTGTACTGGC
TGGTCAGGCCTCCCAGCTAAATGCTGTGGTGGACTTGCAGGACAGAAACAC
CGAACTGTCCTACCAGCTCTTGCTTGACTCTCTGGGTGACAGAACCAGGTA
TTTCAGTATGTGGAATCAGGCGGTGGACAGCTATGACCCCGATGTGCGCAT
TATTGAAAATCACGGTGTGGAGGATGAACTCCCCAATTATTGCTTCCCTTTG
AATGGTGTAGGCTTTACAGATACTTACCAGGGTGTTAAAGTTAAGACAGATA
CAGCCGCTACTGGTACCAATGGAACGCAGTGGGACAAAGATGATACCACAG
TCAGCACTGCCAATGAGATCCACTCAGGCAATCCTTTCGCCATGGAGATCA
ACATCCAGGCCAACCTGTGGCGGAACTTCCTCTACGCGAACGTGGCGCTGT
ACCTGCCCGACTCCTACAAGTACACGCCGGCCAACATCACGCTGCCGACCA
ACACCAACACCTACGATTACATGAACGGCCGCGTGGTGGCGCCCTCGCTG
GTGGACGCCTACATCAACATCGGGGCGCGCTGGTCGCTGGACCCCATGGA
CAACGTCAACCCCTTCAACCACCACCGCAACGCGGGCCTGCGCTACCGCT
CCATGCTCCTGGGCAACGGGCGCTACGTGCCCTTCCACATCCAGGTGCCC
CAAAAGTTTTTCGCCATCAAGAGCCTCCTGCTCCTGCCCGGGTCCTACACC
TACGAGTGGAACTTCCGCAAGGACGTCAACATGATCCTGCAGAGCTCCCTC
GGCAACGACCTGCGCACGGACGGGGCCTCCATCGCCTTCACCAGCATCAA
CCTCTACGCCACCTTCTTCCCCATGGCGCACAACACCGCCTCCACGCTCGA
GGCCATGCTGCGCAACGACACCAACGACCAGTCCTTCAACGACTACCTCTC
GGCGGCCAACATGCTCTACCCCATCCCGGCCAACGCCACCAACGTGCCCA
TCTCCATCCCCTCGCGCAACTGGGCCGCCTTCCGCGGATGGTCCTTCACGC
GCCTCAAGACCCGCGAGACGCCCTCGCTCGGCTCCGGGTTCGACCCCTAC
TTCGTCTACTCGGGCTCCATCCCCTACCTCGACGGCACCTTCTACCTCAAC
CACACCTTCAAGAAGGTCTCCATCACCTTCGACTCCTCCGTCAGCTGGCCC
GGCAACGACCGCCTCCTGACGCCCAACGAGTTCGAAATCAAGCGCACCGT
CGACGGAGAGGGATACAACGTGGCCCAGTGCAACATGACCAAGGACTGGT
TCCTGGTCCAGATGCTGGCCCACTACAACATCGGCTACCAGGGCTTCTACG
TGCCCGAGGGCTACAAGGACCGCATGTACTCCTTCTTCCGCAACTTCCAGC
CCATGAGCCGCCAGGTCGTGGACGAGGTCAACTACAAGGACTACCAGGCC
GTCACCCTGGCCTACCAGCACAACAACTCGGGCTTCGTCGGCTACCTCGCG
CCCACCATGCGCCAGGGCCAGCCCTACCCCGCCAACTACCCCTACCCGCT
CATCGGCAAGAGCGCCGTCGCCAGCGTCACCCAGAAAAAGTTCCTCTGCG
ACCGGGTCATGTGGCGCATCCCCTTCTCCAGCAACTTCATGTCCATGGGCG
CGCTCACCGACCTCGGCCAGAACATGCTCTACGCCAACTCCGCCCACGCG
CTAGACATGAATTTCGAAGTCGACCCCATGGATGAGTCCACCCTTCTCTATG
TTGTCTTCGAAGTCTTCGACGTCGTCCGAGTGCACCAGCCCCACCGCGGCG
TCATCGAGGCCGTCTACCTGCGCACGCCCTTCTCGGCCGGCAACGCCACC
ACCTAAAGCCCCGCTCTTGCTTCTTGCAAGATGACGGCCTGTGGCTCCGGC
GAGCAGGAGCTCAGGGCCATCCTCCGCGACCTGGGCTGCGGGCCCTGCTT
CCTGGGCACCTTCGACAAGCGCTTCCCGGGATTCATGGCCCCGCACAAGC
TGGCCTGCGCCATCGTCAACACGGCCGGCCGCGAGACCGGGGGCGAGCA
CTGGCTGGCCTTCGCCTGGAACCCGCGCTCCCACACCTGCTACCTCTTCGA
CCCCTTCGGGTTCTCGGACGAGCGCCTCAAGCAGATCTACCAGTTCGAGTA
CGAGGGCCTGCTGCGCCGCAGCGCCCTGGCCACCGAGGACCGCTGCATC
ACCCTGGAAAAGTCCACCCAGACCGTGCAGGGTCCGCGCTCGGCCGCCTG
CGGGCTCTTCTGCTGCATGTTCCTGCACGCCTTCGTGCACTGGCCCGACCG
CCCCATGGACAAGAACCCCACCATGAACTTGCTGACGGGGGTGCCCAACG
GCATGCTCCAGTCGCCCCAGGTGGAACCCACCCTGCGCCGCAACCAGGAG
GCGCTCTACCGCTTCCTCAACGCCCACTCCGCCTACTTTCGCTCCCACCGC
GCGCGCATCGAGAAGGCCACCGCCTTCGACCGCATGAATCAAGACATGTAA
ACTGTGTGTATGTGAATGCTTTATTCATCATAATAAACAGCACATGTTTATGC
CACCTTCTCTGAGGCTCTGACTTTATTTAGAAATCGAAGGGGTTCTGCCGGC
TCTCGGCGTGCCCCGCGGGCAGGGATACGTTGCGGAACTGGTACTTGGGC
AGCCACTTGAACTCGGGGATCAGCAGCTTCGGCACGGGGAGGTCGGGGAA
CGAGTCGCTCCACAGCTTGCGCGTGAGTTGCAGGGCGCCCAGCAGGTCGG
GCGCGGAGATCTTGAAATCGCAGTTGGGACCCGCGTTCTGCGCGCGAGAG
TTGCGGTACACGGGGTTGCAGCACTGGAACACCATCAGGGCCGGGTGCTT
CACGCTCGCCAGCACCGTCGCGTCGGTGATGCCCTCCACGTCCAGATCCT
CGGCGTTGGCCATCCCGAAGGGGGTCATCTTGCAGGTCTGCCGCCCCATG
CTGGGCACGCAGCCGGGCTTGTGGTTGCAATCGCAGTGCAGGGGGATCAG
CATCATCTGAGCCTGCTCGGAGCTCATGCCCGGGTACATGGCCTTCATGAA
AGCCTCCAGCTGGCGGAAGGCCTGCTGCGCCTTGCCGCCCTCGGTGAAGA
AGACCCCACAGGACTTGCTAGAGAACTGGTTGGTGGCGCAGCCCGCGTCG
TGCACGCAGCAGCGCGCGTCGTTGTTGGCCAGCTGCACCACGCTGCGCCC
CCAGCGGTTCTGGGTGATCTTGGCCCGGTCGGGGTTCTCCTTCAGCGCGC
GCTGCCCGTTCTCGCTCGCCACATCCATCTCGATCGTGTGCTCCTTCTGGA
TCATCACGGTCCCGTGCAGGCACCGCAGCTTGCCCTCGGCCTCGGTGCAC
CCGTGCAGCCACAGCGCGCAGCCGGTGCACTCCCAGTTCTTGTGGGCGAT
CTGGGAGTGCGAGTGCACGAAGCCCTGCAGGAAGCGGCCCATCATCGTGG
TCAGGGTCTTGTTGCTGGTGAAGGTCAGCGGGATGCCGCGGTGCTCCTCG
TTCACATACAGGTGGCAGATGCGGCGGTACACCTCGCCCTGCTCGGGCAT
CAGCTGGAAGGCGGACTTCAGGTCGCTCTCCACGCGGTACCGCTCCATCA
GCAGCGTCATCACTTCCATGCCCTTCTCCCAGGCCGAAACGATCGGCAGGC
TCAGGGGGTTCTTCACCGTCATCTTAGTCGCCGCCGCCGAAGTCAGGGGG
TCGTTCTCGTCCAGGGTCTCAAACACTCGCTTGCCGTCCTTCTCGGTGATG
CGCACGGGGGGAAAGCTGAAGCCCACGGCCGCCAGCTCCTCCTCGGCCT
GCCTTTCGTCCTCGCTGTCCTGGCTGATGTCTTGCAAAGGCACATGCTTGG
TCTTGCGGGGTTTCTTTTTGGGCGGCAGAGGCGGCGGCGGAGACGTGCTG
GGCGAGCGCGAGTTCTCGCTCACCACGACTATTTCTTCTTCTTGGCCGTCG
TCCGAGACCACGCGGCGGTAGGCATGCCTCTTCTGGGGCAGAGGCGGAG
GCGACGGGCTCTCGCGGTTCGGCGGGCGGCTGGCAGAGCCCCTTCCGCG
TTCGGGGGTGCGCTCCTGGCGGCGCTGCTCTGACTGACTTCCTCCGCGGC
CGGCCATTGTGTTCTCCTAGGGAGCAACAAGCATGGAGACTCAGCCATCGT
CGCCAACATCGCCATCTGCCCCCGCCGCCGACGAGAACCAGCAGCAGCAG
AATGAAAGCTTAACCGCCCCGCCGCCCAGCCCCACCTCCGACGCCGCCGC
GGCCCCAGACATGCAAGAGATGGAGGAATCCATCGAGATTGACCTGGGCTA
CGTGACGCCCGCGGAGCACGAGGAGGAGCTGGCAGCGCGCTTTTCAGCC
CCGGAAGAGAACCACCAAGAGCAGCCAGAGCAGGAAGCAGAGAGCGAGCA
GCAGCAGGCTGGGCTCGAGCATGGCGACTACCTGAGCGGGGCAGAGGAC
GTGCTCATCAAGCATCTGGCCCGCCAAAGCATCATCGTCAAGGACGCGCTG
CTCGACCGCGCCGAGGTGCCCCTCAGCGTGGCGGAGCTCAGCCGCGCCT
ACGAGCGCAACCTCTTCTCGCCGCGCGTGCCCCCCAAGCGCCAGCCCAAC
GGCACCTGCGAGCCCAACCCGCGCCTCAACTTCTACCCGGTCTTCGCGGT
GCCCGAGGCCCTGGCCACCTACCACCTCTTTTTCAAGAACCAAAGGATCCC
CGTCTCCTGCCGCGCCAACCGCACCCGCGCCGACGCCCTGCTCAACCTGG
GTCCCGGCGCCCGCCTACCTGATATCACCTCCTTGGAAGAGGTTCCCAAGA
TCTTCGAGGGTCTGGGCAGCGACGAGACTCGGGCCGCGAACGCTCTGCAA
GGAAGCGGAGAGGAGCATGAGCACCACAGCGCCCTGGTGGAGTTGGAAG
GCGACAACGCGCGCCTGGCGGTGCTCAAGCGCACGGTCGAGCTGACCCAC
TTCGCCTACCCGGCGCTCAACCTGCCCCCCAAGGTCATGAGCGCCGTCAT
GGACCAGGTGCTCATCAAGCGCGCCTCGCCCCTCTCAGAGGAGGAGATGC
AGGACCCCGAGAGCTCGGACGAGGGCAAGCCCGTGGTCAGCGACGAGCA
GCTGGCGCGCTGGCTGGGAGCGAGCAGCACCCCCCAGAGCCTGGAAGAG
CGGCGCAAGCTCATGATGGCCGTGGTCCTGGTGACCGTGGAGCTGGAGTG
TCTGCGCCGCTTCTTCGCCGACGCGGAGACCCTGCGCAAGGTCGAGGAGA
ACCTGCACTACCTCTTCAGGCACGGGTTCGTGCGCCAGGCCTGCAAGATCT
CCAACGTGGAGCTGACCAACCTGGTCTCCTACATGGGCATCCTGCACGAGA
ACCGCCTGGGGCAGAACGTGCTGCACACCACCCTGCGCGGGGAGGCCCG
CCGCGACTACATCCGCGACTGCGTCTACCTGTACCTCTGCCACACCTGGCA
GACGGGCATGGGCGTGTGGCAGCAGTGCCTGGAGGAGCAGAACCTGAAAG
AGCTCTGCAAGCTCCTGCAGAAGAACCTCAAGGCCCTGTGGACCGGGTTC
GACGAGCGCACCACCGCCTCGGACCTGGCCGACCTCATCTTCCCCGAGCG
CCTGCGGCTGACGCTGCGCAACGGGCTGCCCGACTTTATGAGCCAAAGCA
TGTTGCAAAACTTTCGCTCTTTCATCCTCGAACGCTCCGGGATCCTGCCCGC
CACCTGCTCCGCACTGCCCTCGGACTTCGTGCCGCTGACCTTCCGCGAGT
GCCCCCCGCCGCTCTGGAGCCACTGCTACTTGCTGCGCCTGGCCAACTAC
CTGGCCTACCACTCGGACGTGATCGAGGACGTCAGCAGCGAGGGTCTGCT
CGAGTGCCACTGCCGCTGCAACCTCTGCACGCCGCACCGCTCCTTGGCCT
GCAACCCCCAGCTGCTGAGCGAGACCCAGATCATCGGCACCTTCGAGTTG
CAAGGCCCCGGCGAGGGCAAGGGGGGTCTCAAACTCACCCCGGGGCTGT
GGACCTCGGCCTACTTGCGCAAGTTCGTGCCCGAGGACTACCATCCCTTCG
AGATCAGGTTCTACGAGGACCAATCCCAGCCGCCCAAGGCCGAGCTGTCG
GCCTGCGTCATCACCCAGGGGGCCATCCTGGCCCAATTGCAAGCCATCCA
GAAATCCCGCCAAGAATTTCTGCTGAAAAAGGGCCACGGGGTCTACTTGGA
CCCCCAGACCGGAGAGGAGCTCAACCCCAGCTTCCCCCAGGATGCCCCGA
GGAAGCAGCAAGAAGCTGAAAGTGGAGCTGCCGCTGCCGCCGGAGGATTT
GGAGGAAGACTGGGAGAGCAGTCAGGCAGAGGAGATGGAAGACTGGGACA
GCACTCAGGCAGAGGAGGACAGCCTGCAAGACAGTCTGGAGGAGGAAGAC
GAGGTGGAGGAGGAGGCAGAGGAAGAAGCAGCCGCCGCCAGACCGTCGT
CCTCGGCGGAGGAGAAAGCAAGCAGCACGGATACCATCTCCGCTCCGGGT
CGGGGTCGCGGCGGCCGGGCCCACAGTAGATGGGACGAGACCGGGCGCT
TCCCGAACCCCACCACCCAGACCGGTAAGAAGGAGCGGCAGGGATACAAG
TCCTGGCGGGGGCACAAAAACGCCATCGTCTCCTGCTTGCAAGCCTGCGG
GGGCAACATCTCCTTCACCCGGCGCTACCTGCTCTTCCACCGCGGGGTGAA
CTTCCCCCGCAACATCTTGCATTACTACCGTCACCTCCACAGCCCCTACTAC
TGTTTCCAAGAAGAGGCAGAAACCCAGCAGCAGCAGCAGAAAACCAGCGG
CAGCAGCAGCAGCTAGAAAATCCACAGCGGCGGCAGGTGGACTGAGGATC
GCGGCGAACGAGCCGGCGCAGACCCGGGAGCTGAGGAACCGGATCTTTC
CCACCCTCTATGCCATCTTCCAGCAGAGTCGGGGGCAGGAGCAGGAACTG
AAAGTCAAGAACCGTTCTCTGCGCTCGCTCACCCGCAGTTGTCTGTATCACA
AGAGCGAAGACCAACTTCAGCGCACTCTCGAGGACGCCGAGGCTCTCTTCA
ACAAGTACTGCGCGCTCACTCTTAAAGAGTAGCCCGCGCCCGCCCACACAC
GGAAAAAGGCGGGAATTACGTCACCACCTGCGCCCTTCGCCCGACCATCAT
CATGAGCAAAGAGATTCCCACGCCTTACATGTGGAGCTACCAGCCCCAGAT
GGGCCTGGCCGCCGGCGCCGCCCAGGACTACTCCACCCGCATGAACTGG
CTCAGTGCCGGGCCCGCGATGATCTCACGGGTGAATGACATCCGCGCCCA
CCGAAACCAGATACTCCTAGAACAGTCAGCGATCACCGCCACGCCCCGCCA
TCACCTTAATCCGCGTAATTGGCCCGCCGCCCTGGTGTACCAGGAAATTCC
CCAGCCCACGACCGTACTACTTCCGCGAGACGCCCAGGCCGAAGTCCAGC
TGACTAACTCAGGTGTCCAGCTGGCCGGCGGCGCCGCCCTGTGTCGTCAC
CGCCCCGCTCAGGGTATAAAGCGGCTGGTGATCCGAGGCAGAGGCACACA
GCTCAACGACGAGGTGGTGAGCTCTTCGCTGGGTCTGCGACCTGACGGAG
TCTTCCAACTCGCCGGATCGGGGAGATCTTCCTTCACGCCTCGTCAGGCCG
TCCTGACTTTGGAGAGTTCGTCCTCGCAGCCCCGCTCGGGCGGCATCGGC
ACTCTCCAGTTCGTGGAGGAGTTCACTCCCTCGGTCTACTTCAACCCCTTCT
CCGGCTCCCCCGGCCACTACCCGGACGAGTTCATCCCGAACTTCGACGCC
ATCAGCGAGTCGGTGGACGGCTACGATTGAATGTCCCATGGTGGCGCGGC
TGACCTAGCTCGGCTTCGACACCTGGACCACTGTTAATTAATCGCCTCTCCT
ACGAGCTCCTGCAGCAGCGCCAGAAGTTCACCTGCCTGGTCGGAGTCAAC
CCCATCGTCATCACCCAGCAGTCGGGCGATACCAAGGGGTGCATCCACTG
CTCCTGCGACTCCCCCGACTGCGTCCACACTCTGATCAAGACCCTCTGCGG
CCTCCGCGACCTCCTCCCCATGAACTAATCACCCCCTTATCCAGTGAAATAA
AGATCATATTGATGATGATTTTACAGAAATAAAGATACAATCATATTGATGAT
TTGAGTTTAATAAAAAATAAAGAATCACTTACTTGAAATCTGATACCAGGTCT
CTGTCCATGTTTTCTGCCAACACCACTTCACTCCCCTCTTCCCAGCTCTGGT
ACTGCAGGCCCCGGCGGGCTGCAAACTTCCTCCACACGCTGAAGGGGATG
TCAAATTCCTCCTGTCCCTCAATCTTCATTTTATCTTCTATCAGATGTCCAAA
AAGCGCGTCCGGGTGGATGATGACTTCGACCCCGTCTACCCCTACGATGCA
GACAACGCACCGACCGTGCCCTTCATCAACCCCCCCTTCGTCTCTTCAGAT
GGATTCCAAGAGAAGCCCCTGGGGGTGCTGTCCCTGCGACTGGCCGACCC
CGTCACCACCAAGAACGGGGAAATCACCCTCAAGCTGGGAGAGGGGGTGG
ACCTCGACTCCTCGGGAAAACTCATCTCCAACACGGCCACCAAGGCCGCCG
CCCCTCTCAGTTTTTCCAACAACACCATTTCCCTTAACATGGATCACCCCTTT
TACACTAAAGATGGAAAATTATCCTTACAAGTTTCTCCACCATTAAATATACT
GAGAACAAGCATTCTAAACACACTAGCTTTAGGTTTTGGATCAGGTTTAGGA
CTCCGTGGCTCTGCCTTGGCAGTACAGTTAGTCTCTCCACTTACATTTGATA
CTGATGGAAACATAAAGCTTACCTTAGACAGAGGTTTGCATGTTACAACAGG
AGATGCAATTGAAAGCAACATAAGCTGGGCTAAAGGTTTAAAATTTGAAGAT
GGAGCCATAGCAACCAACATTGGAAATGGGTTAGAGTTTGGAAGCAGTAGT
ACAGAAACAGGTGTTGATGATGCTTACCCAATCCAAGTTAAACTTGGATCTG
GCCTTAGCTTTGACAGTACAGGAGCCATAATGGCTGGTAACAAAGAAGACG
ATAAACTCACTTTGTGGACAACACCTGATCCATCGCCAAACTGTCAAATACT
CGCAGAAAATGATGCAAAACTAACACTTTGCTTGACTAAATGTGGTAGTCAA
ATACTGGCCACTGTGTCAGTCTTAGTTGTAGGAAGTGGAAACCTAAACCCCA
TTACTGGCACCGTAAGCAGTGCTCAGGTGTTTCTACGTTTTGATGCAAACGG
TGTTCTTTTAACAGAACATTCTACACTAAAAAAATACTGGGGGTATAGGCAG
GGAGATAGCATAGATGGCACTCCATATACCAATGCTGTAGGATTCATGCCCA
ATTTAAAAGCTTATCCAAAGTCACAAAGTTCTACTACTAAAAATAATATAGTA
GGGCAAGTATACATGAATGGAGATGTTTCAAAACCTATGCTTCTCACTATAA
CCCTCAATGGTACTGATGACAGCAACAGTACATATTCAATGTCATTTTCATAC
ACCTGGACTAATGGAAGCTATGTTGGAGCAACATTTGGGGCTAACTCTTATA
CCTTCTCATACATCGCCCAAGAATGAACACTGTATCCCACCCTGCATGCCAA
CCCTTCCCACCCCACTCTGTGGAAAAAACTCTGAAACACAAAATAAAATAAA
GTTCAAGTGTTTTATTGATTCAACAGTTTTACAGGATTCGAGCAGTTATTTTT
CCTCCACCCTCCCAGGACATGGAATACACCACCCTCTCCCCCCGCACAGCC
TTGAACATCTGAATGCCATTGGTGATGGACATGCTTTTGGTCTCCACGTTCC
ACACAGTTTCAGAGCGAGCCAGTCTCGGGTCGGTCAGGGAGATGAAACCCT
CCGGGCACAATTGGGAGAAGTACTCGCCTACATGGGGGTAGAGTCATAATC
GTGCATCAGGATAGGGCGGTGGTGCTGCAGCAGCGCGCGAATAAACTGCT
GCCGCCGCCGCTCCGTCCTGCAGGAATACAACATGGCAGTGGTCTCCTCA
GCGATGATTCGCACCGCCCGCAGCATAAGGCGCCTTGTCCTCCGGGCACA
GCAGCGCACCCTGATCTCACTTAAATCAGCACAGTAACTGCAGCACAGCAC
CACAATATTGTTCAAAATCCCACAGTGCAAGGCGCTGTATCCAAAGCTCATG
GCGGGGACCACAGAACCCACGTGGCCATCATACCACAAGCGCAGGTAGAT
TAAGTGGCGACCCCTCATAAACACGCTGGACATAAACATTACCTCTTTTGGC
ATGTTGTAATTCACCACCTCCCGGTACCATATAAACCTCTGATTAAACATGG
CGCCATCCACCACCATCCTAAACCAGCTGGCCAAAACCTGCCCGCCGGCTA
TACACTGCAGGGAACCGGGACTGGAACAATGACAGTGGAGAGCCCAGGAC
TCGTAACCATGGATCATCATGCTCGTCATGATATCAATGTTGGCACAACACA
GGCACACGTGCATACACTTCCTCAGGATTACAAGCTCCTCCCGCGTTAGAA
CCATATCCCAGGGAACAACCCATTCCTGAATCAGCGTAAATCCCACACTGCA
GGGAAGACCTCGCACGTAACTCACGTTGTGCATTGTCAAAGTGTTACATTCG
GGCAGCAGCGGATGATCCTCCAGTATGGTAGCGCGGGTTTCTGTCTCAAAA
GGAGGTAGACGATCCCTACTGTACGGAGTGCGCCGAGACAACCGAGATCG
TGTTGGTCGTAGTGTCATGCCAAATGGAACGCCGGACGTAGTCATATTTCCT
GAAGTCTTGGCGCGCCAAAGTCTAGAAGCGGTCCATAGCTTACCGAGCGG
CAGCAGCAGCGGCACACAACAGGCGCAAGAGTCAGAGAAAAGACTGAGCT
CTAACCTGTCCGCCCGCTCTCTGCTCAATATATAGCCCCAGATCTACACTGA
CGTAAAGGCCAAAGTCTAAAAATACCCGCCAAATAATCACACACGCCCAGC
ACACGCCCAGAAACCGGTGACACACTCAGAAAAATACGCGCACTTCCTCAA
ACGGCCAAACTGCCGTCATTTCCGGGTTCCCACGCTACGTCATCAAAACAC
GACTTTCAAATTCCGTCGACCGTTAAAAACATCACCCGCCCCGCCCCTAAC
GGTCGCCGCTCCCGCAGCCAATCACCTTCCTCCCTCCCCAAATTCAAACAG
CTCATTTGCATATTAACGCGCACCAAAAGTTTGAGGTATATTATTGATGATGG
GTTT

### LIST OF FIGURES

FIG. 1: Modulation of immune responses to AdHu5 backbone by CpG 1826.

C57BL/6 mice were immunised intradermally, i.d. with AdHu5 PfM115 (5×10¹⁰ viral particles, v.p.), mixed with PBS alone (nil) or with 50 μg CpG1826 (CpG). Sera for antibody ELISA were collected on day 14-18. (A) Total IgG titres measured by ELISA against AdHu5 GFP. Responses of individual mice and or GMT responses are shown. (B) Results from (A) are shown as end-point log 10 titres plotted against the corresponding GST-PfMSP-1₁₉ specific IgG end-point log 10 titres for each mouse. ** Differs from AdHu5 PfM115 alone, P 0.01.

FIG. 2: Modulation of immune responses to vaccination with AdHu5 PfM115 by poly (I:C), CpG ODN or imiquimod. C57BL/6 mice (n=6 per group) were immunised i.d. with AdHu5 PfM115 (5×10¹⁰ v.p.) on day 0. Subsequently, 30 μg subcutaneous poly (1:0) or 20 mg topical imiquimod (IMQ) were administered on days 0, 2, 5, 7 and 9. In one group the first dose of imiquimod was applied one hour later than AdHu5 PfM115 administration (IMQ+); 50 μg CpG ODN 1826 mixed with AdHu5 PfM115 was administered i.d. once only (CpG). Spleens for ICS and sera for antibody ELISA were collected on day 14. Responses of individual mice and median (or GMT) responses are shown. (A) CD8+ responses to four peptides 86, 100, 149 & 215 were summed. (B) CD4+ responses to peptide 188. (C) Total IgG titres measured by ELISA against ETSR GST-PfMSP1₁₉. No responses were seen against GST controls.

* Differs from vector alone (Nil), P≦0.05

** Differs from vector alone (Nil), P≦0.01

FIG. 3: Modulation of immune responses to MVA PyMSP1₄₂ immunisation by Carbopol adjuvant. BALB/c mice (n=6 per group) were immunised i.m. with MVA PyMSP1₄₂ (10⁶ or 10⁷ pfu) formulated with or without Carbpol adjuvant. Carbopol powder was mixed with water and adjusted to pH 7.2 with NaOH. MVA vaccine in PBS was formulated with 0.25% Carbopol using a positive displacement pipette. Mice were immunised i.m. on d0, and spleens harvested on d14. CD8+ and CD4+IFN-γ+ T cell responses in the spleen were assessed by ICS against a pool of PyMSP1₃₃ overlapping peptides. Results show the mean (A) total number per spleen or (B) % frequency antigen specific CD8+ or CD4+IFN-γ+ T cells±SEM (n=5 mice per group).

FIG. 4: CD4 and CD8 peripheral T cell responses to malaria AMA1 antigen following vaccination with ISCOM Matrix adjuvanted vaccine. BALB/c mice (n=5 per group) were vaccinated i.m. with AdCh63-AMA1 (5×10⁸ viral particles per dose), AMA-1 protein (10 μg/dose) with or without ISCOM Matrix M (12 μg/dose). Peripheral blood CD4 (a and b) and CD8 (c) T cell responses were assessed following one, two or three homologous vaccinations, corresponding to time-points of 3, 10 and 18 weeks, respectively. Mouse PBMCs were stimulated in an overnight IFN-γ ELISpot with the following AMA1 peptides used at 5 μg/ml final concentration: VFGKGIIIENSKTTF and NKKIIAPRIFISDDK (P31 and P95, respectively, CD4 T cells epitopes) and KYVKNLDELTLCSRH (P42, CD8 T cell epitope).

FIG. 5: CD4 and CD8 T cell responses in the spleen to malaria AMA1 antigen following vaccination with ISCOM Matrix adjuvanted vaccine. BALB/c mice (n=5 per group) were vaccinated I.M. with AdCh63-AMA1 vectored vaccine (5×10⁸ viral particles per dose), AMA-1 protein (10 μg/dose) with or without saponin-containing adjuvant ISCOM Matrix M (12 μg/dose). Spleen CD4 (a) and CD8 (b) T cell responses were assessed at the final time-point, 6 months from the first vaccination (2 months after the last vaccination). Mouse PBMCs were stimulated in an overnight IFN-γ ELISpot with the following AMA1 peptides used at 5 μg/ml final concentration: VFGKGIIIENSKTTF and NKKIIAPRIFISDDK (P31 and P95, respectively, CD4 T cells epitopes) and KYVKNLDELTLCSRH (P42, CD8 T cell epitope). Following assay development (ELISpot kit, Mabtech; AP Conjugate sub kit, Bio-Rad), spots were counted using AID ELISpot reader (Autoimmun Diagnostika GmbH) and calculated as spot forming units per million PBMCs.

FIG. 6: Antibody responses to malaria AMA1 antigen following vaccination with ISCOM Matrix adjuvanted vaccine. BALB/c mice (n=5 per group) were vaccinated I.M. with AdCh63-AMA1 vectored vaccine (5×10⁸ viral particles per dose), AMA-1 protein (10 μg/dose) with or without saponin-containing adjuvant ISCOM Matrix M (12 μg/dose). Antibody responses shown here were assessed 2 weeks after the priming vaccination (a), a day before the second vaccination (b), two weeks after the second vaccination (c), a day before third vaccination (d), two weeks after third vaccination (e) and 6 weeks after third vaccination (f). Total IgG responses to whole AMA1 protein were assayed using a standard ELISA protocol and absorption at 405 nm plates measured using a Microplate reader (Bio-Rad).

FIG. 7. Spleen CD4 and CD8 antigen-specific T cell responses following vaccination with AdCh63-AMA1 vaccine with or without ISCOM Matrix adjuvant. BALB/c mice (n=5 per group) were vaccinated I.M. with AdCh63-AMA1 vectored vaccine (5×10⁸ viral particles per dose) with or without ISCOM Matrix M (12 μg/dose). Spleen T cell responses against CD4 (a) and CD8 (b) epitopes in AMA-1 were assessed two weeks after immunisation. Mouse splenocytes were stimulated in an overnight IFN-γ ELISpot with the following AMA1 peptides used at 5 μg/ml final concentration: VFGKGIIIENSKTTF and NKKIIAPRIFISDDK (P31 and P95, respectively, CD4 T cells epitopes) and KYVKNLDELTLCSRH (P42, CD8 T cell epitope). Following assay development (ELISpot kit, Mabtech; AP Conjugate sub kit, Bio-Rad), spots were counted using AID ELISpot reader (Autoimmun Diagnostika GmbH) and calculated per million PBMCs.

FIG. 8. Spleen CD4 and CD8 T cell responses to viral vectored AdCh63-AMA-1 vaccine combined with water and oil emulsions. BALB/c mice (n=5 per group) were vaccinated I.M. with AdCh63-AMA1 vectored vaccine (5×10⁸ viral particles per dose) alone or combined with Montanide ISA 720 (ratio of Ag:Adjuvant=3:7 in total vaccination volume of 50 ml, homogenized to a stable emulsion), ISA 206 (ratio 1:1, vortexed) and Emulsigen (ratio of Ag:Adjuvant=8:2, vortexed). Spleen T cell responses against CD4 (a) and CD8 (b) epitopes in AMA-1 were assessed two weeks after immunisation. Mouse splenocytes were stimulated in an overnight IFN-γ ELISpot with the following AMA1 peptides used at 5 μg/ml final concentration: VFGKGIIIENSKTTF and NKKIIAPRIFISDDK (P31 and P95, respectively, CD4 T cells epitopes) and KYVKNLDELTLCSRH (P42, CD8 T cell epitope). Following assay development (ELISpot kit, Mabtech; AP Conjugate sub kit, Bio-Rad), spots were counted using AID ELISpot reader (Autoimmun Diagnostika GmbH) and calculated per million PBMCs.

FIG. 9: Cytokine responses following vaccination with Ad-ME.TRAP adjuvanted with ISCOM Matrix. BALB/c mice (n=6) were immunized intradermally into the ear with 5×10⁹ vp/mouse Ad-ME.TRAP. A control group received the vaccine in PBS. The adjuvanted vaccine resulted in a non-significant increase of the frequencies of antigen-specific CD8 T-cells producing IFN-γ, TNF-α and IL-2 in blood, as well as the expression of the degranulation marker CD107a.

FIG. 10: The vaccinated mice were challenged with malaria intravenously with 1,000 sporozoites per mouse. Incidence of parasitaemia was analysed by visual inspection of blood smears starting from day 5 post-challenge and vaccine efficacy measured as a delay in reaching detectable blood parasitaemia. The addition of ISCOM Matrix resulted in a 2-day delay in the development of parasitaemia in the blood as compared to the adenoviral vaccine alone.

FIG. 11: Comparison of responses of BALB/c mice to three component sequential and two-stage mixed-component regimes. Comparison of responses of BALB/c mice to three component sequential, two-stage mixed-component, and two component sequential regimes. n=6/group. Mice receiving three vaccinations were primed at day 0, with subsequent boosts on days 97 and 154. Mice receiving two vaccinations received these on days 97 and 154. This permitted all results depicted in this figure to be obtained from synchronous assays 14 days after final vaccination. The abbreviations ‘A’, ‘M’ and ‘P’ are used in place of ‘AdCh63’, ‘MVA’ and ‘protein’ respectively. A dash is used to indicate separate sequential vaccinations whereas parentheses and a + sign indicates mixed vaccinations—for example, ‘A-P’ indicates AdCh63 followed by protein, whereas ‘(A+P)’ indicates mixed adenovirus and protein given simultaneously at the same site. The doses used were 10¹⁰ virus particles (vp) for AdCh63 PfM128 [Goodman A L, Epp C, Moss D, et al. Infect Immun. 2010 Aug. 16.], 10⁷ plaque forming units (pfu) for MVA PfM128 and 20 μg for protein MSP1₁₉ [Morgan, W. D. et al. J Mol Biol 289, 113-122 (1999).]. Protein in endotoxin-free PBS was mixed manually in a syringe immediately prior to immunization with Montanide ISA720 adjuvant (SEPPIC, France) as previously described [Arevalo-Herrera, M. et al. Am J Trop Med Hyg 73, 25-31 (2005).]. Graphs plot individual values (symbols) and group mean (line).

Left panel: IFNγ⁺ CD8⁺ T cell responses assessed by ICS

Right panel: Total IgG responses assessed by ELISA

FIG. 12: CD CD8 peripheral T cell responses to Pb9 peptide from ME.TRAP following three vaccinations with MVA ME.TRAP adjuvanted with ISCOM Matrix. BALB/c mice (n=5 per group) were vaccinated intradermally 3 times with intervals of 2 weeks between immunisations, with MVA ME.TRAP (1×10⁶ pfu per dose) and MVA ME.TRAP (1×10⁶ pfu per dose) adjuvanted with ISCOM Matrix (12 μg/dose). Peripheral CD8 T cell responses were assessed 2 weeks after the last vaccination. Mouse PBMCs were stimulated for 5 hours with Pb9 peptide (SYIPSAEKI) at a final concentration of 1 μg/ml. A) Frequencies of CD8+ T-cells producing A) IFN-γ; B) TNF-α and C) IL-2 upon peptide stimulation. D) Multi-functional CD8+ responses in the same experiment. This analysis was performed by taking into account the expression of the 3 cytokines from A, B and C from individual cells. Analysis was done using the SPICE software kindly provided by the NIH. Light-grey: one cytokine; medium-grey: (any) two cytokines, dark-grey: all three cytokines.

FIG. 13: Protein in various adjuvants, including Alum, can boost an adenovirus-primed response to achieve high antibody titres.

FIG. 14: Higher dose of ISCOM Matrix enhances protective efficacy of Ad-vectored malaria vaccine.

Vaccinated mice were challenged with malaria intravenously with 1,000 sporozoites per mouse. Incidence of parasitaemia was analysed by visual inspection of blood smears starting from day 5 post-challenge and vaccine efficacy measured as percent animal survival. Addition of 24 μg ISCOM Matrix to the Ad-ME.TRAP vaccine resulted in a higher proportion of surviving animals as compared to the adenoviral vaccine alone.

FIG. 15: ISCOM Matrix significantly increases the proportion of Ag-specific TCM in peripheral blood.

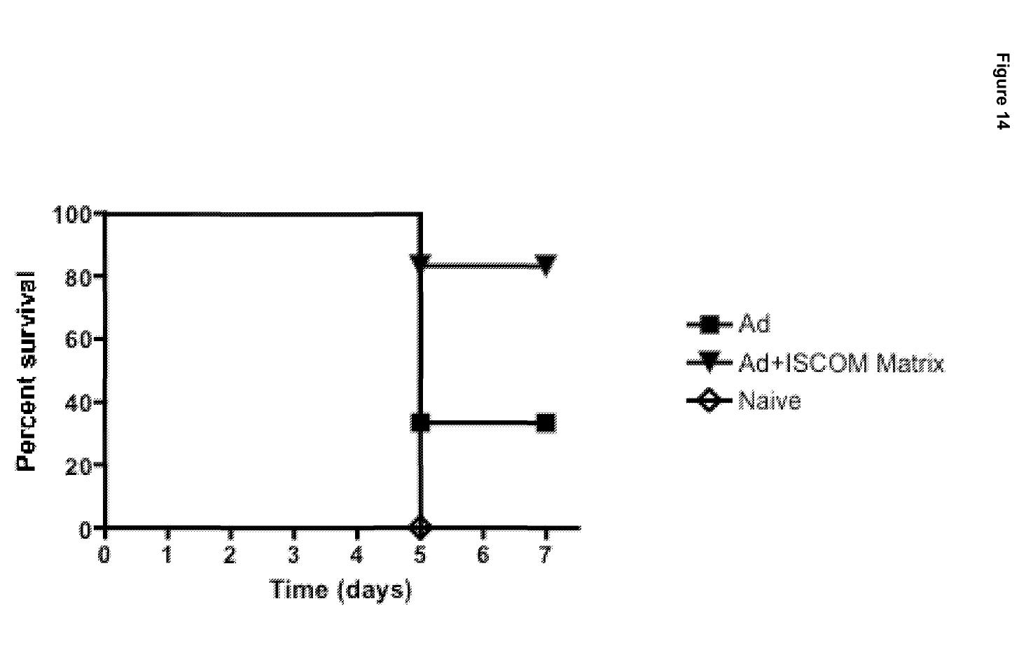

Peripheral blood (A, B and C) and spleen (D, E and F) from mice vaccinated with Ad-ME.TRAP with or without ISCOM Matrix were examined for the proportion of antigen-specific TCM, TEM and TE cell subsets, respectively. The central memory T cells, which are associated with the longevity of vaccine efficacy, were found to be significantly increased in the peripheral blood when ISCOM Matrix was added to the Ad-ME.TRAP vaccine (A), supporting the enhanced survival observed when this adjuvant was added at a higher dose to the Ad-ME.TRAP vaccine (shown in FIG. 14). For each graph, the x-axis shows (from left to right) AdC63 and AdC63+ISCOM Matrix. The y-axis shows % CD8+Pb9+CD62L−CD127−.

### EXAMPLES

### Example 1

This example describes the materials and methods used in the following examples.

Materials and Methods

Animals and Immunizations

All procedures were performed in accordance with the terms of the UK Animals (Scientific Procedures) Act Project Licence and were approved by the University of Oxford Animal Care and Ethical Review Committee. 5-6 wk old female BALB/c (H-2^{d}) and C57BL/6 (H-2^{b}) mice (Harlan Laboratories, Oxfordshire, UK), were anesthetized before immunization with medetomidine (Domitor, Pfizer) and ketamine (Ketaset, Fort Dodge) and revived subsequently with Antisedan reversal agent (Pfizer). All immunizations were administered intramuscularly (i.m.) unless otherwise specified, with vaccine divided equally into each musculus tibialis.

The creation of simian adenovirus 63 (AdCh63) and modified vaccinia virus Ankara (MVA) vectors encoding the PfM128 antigen is described elsewhere [Goodman A L, Epp C, Moss D, et al. Infect Immun. 2010 Aug. 16.]. Briefly, this antigen is a bi-allelic fusion incorporating the MSP1₄₂ antigen from the K1/Wellcome and 3D7/MAD20 P. falciparum strains fused in tandem alongside four blocks of conserved sequence from the remainder of the 3D7 strain MSP1 molecule (blocks 1, 3, 5 and 12). Note that this AdCh63 vector has deletions in both the E1 region and the E3 region ensuring replication incompetence in almost all mammalian cells and increasing the size of the insert that can be used to >5 kb. The MVA used in the current study differs from the previously published vector [Draper S J, Moore A C, Goodman A L, Long C A, Holder A A, Gilbert S C, et al. Nat Med 2008 August; 14(8):819-21.] in that it lacked the green fluorescent protein (GFP) marker. To generate the markerless MVA expressing PfM128, the antigen was cloned into a transient-dominant shuttle vector plasmid such that PfM128 was expressed from the vaccinia P7.5 promoter, and inserted into the TK locus of MVA. The plasmid also expresses a GFP marker [Falkner F G, Moss B. Journal of Virology 1990; 64(6):3108-11.]. This plasmid was transfected into chicken embryo fibroblast cells (CEFs) infected with MVA expressing red fluorescent protein (RFP), as previously described [Draper S J, Moore A C, Goodman A L, Long C A, Holder A A, Gilbert S C, et al. Nat Med 2008 August; 14(8):819-21.]. Recombinant MVAs were generated by homologous recombination between regions of homology at the TK locus of MVA and in the plasmid shuttle vector. Unstable intermediate recombinants expressing RFP and GFP were selected using a MoFlo cell-sorter (Beckman Coulter, USA) and plated out on CEFs. After 2-3 passages, further recombination between the repeated TK flanking regions results in either reversion to the starting virus (MVA-RFP) or formation of the markerless recombinant virus MVA-PfM128. White plaques (expressing neither RFP nor GFP) were picked and purified. Presence of the PfM128 antigen at the TK locus was confirmed by sequencing and PCR.

The protein vaccine used was mono-allelic Wellcome strain MSP1₁₉ expressed in the yeast Pichia pastoris (kindly provided by A Holder, NIMR, London) [Morgan W D, Birdsall B, Frenkiel T A, Gradwell M G, Burghaus P A, Syed S E, et al. J Mol Biol 1999 May 28; 289(1):113-22.]. The full sequence of this antigen is represented within the viral vector vaccines. Protein in endotoxin-free PBS was mixed manually in a syringe immediately prior to immunization with Montanide ISA720 adjuvant (SEPPIC, France), in the ratio 3:7 as previously described [Arevalo-Herrera M, Castellanos A, Yazdani S S, Shakri A R, Chitnis C E, Dominik R, et al. Am J Trop Med Hyg 2005 November; 73(5 Suppl):25-31.]. Where applicable, viral vectored vaccines were incorporated in the protein-PBS fraction of this mixture.

BALB/c mice were vaccinated at 8 or 14 week intervals with doses as follows (unless otherwise specified): 10¹⁰ virus particles (vp) for AdCh63; 10⁷ plaque forming units (pfu) for MVA; and 20 μg of protein. C57BL/6 mice were vaccinated at 8 week intervals with 10⁸ vp AdCh63, 10⁶ pfu MVA, or 5 μg protein. Blood was obtained for immunological studies using tail bleeds two weeks after each immunization and at later time points as described.

Ex-Vivo IFNγ and Splenic Antibody-Secreting Cell ELISPOT

Ex-vivo IFNγ enzyme linked immunosorbent assays (ELISPOT) were performed as previously described [Moore A C, Gallimore A, Draper S J, Watkins K R, Gilbert S C, Hill A V. J Immunol 2005 Dec. 1; 175(11):7264-73.], using peptides appropriate to the mouse strain as follows: either the overlapping peptides 90 and 91 (NKEKRDKFLSSYNYI and DKFLSSYNYIKDSID) which comprise the immunodominant CD8⁺ T cell epitope in PfMSP1₃₃ (Wellcome allele) in BALB/c mice; or the PfMSP1₁₉ (3D7 allele)-derived peptide 215 (TKPDSYPLFDGIFCS) recognised by CD8⁺ T cells from C57BL/6 mice[5].

Antigen-specific splenic antibody secreting cells (ASCs) were measured as previously described [Slifka M K, Ahmed R. J Immunol Methods 1996 Nov. 29; 199(1):37-46.]. In brief, nitrocellulose bottomed 96-well Multiscreen HA filtration plates (Millipore, UK) were coated with 5 μg/ml P. falciparum MSP-1₁₉ (Wellcome/FVO allele, expressed in Pichia) [Morgan W D, Birdsall B, Frenkiel T A, Gradwell M G, Burghaus P A, Syed S E, et al. J Mol Biol 1999 May 28; 289(1):113-22.] and incubated overnight at 4° C. Plates were washed twice with PBS and blocked for 1 h at 37° C., 5% CO₂ with D10 (MEM α-modification, 10% Fetal Calf Serum, 4 mM L-glutamine, 100 U/mL penicillin and 100 ug/mL streptomycin (all from Sigma, UK); and 50 μm 2-mercaptoethanol (Gibco)). 5×10⁵ splenocytes were plated onto the pre-coated ELISPOT plate per replicate well and serially diluted. Plates were incubated for 5 h at 37° C., 5% CO₂. Following incubation plates were washed twice with PBS and incubated overnight at 4° C. with biotinylated anti-mouse γ-chain specific IgG antibody (CALTAG, CA).

Assays were developed using colour developing agents (Bio-Rad AP conjugate substrate kit) that were filtered through a 0.2 μm filter (Sartorius, UK). ELISPOT plates were counted using AID plate reader software (AID, Cadama Medical) and counts were visually confirmed. No spots were observed in control wells containing splenocytes but no coating antigen.

Intracellular Cytokine Staining

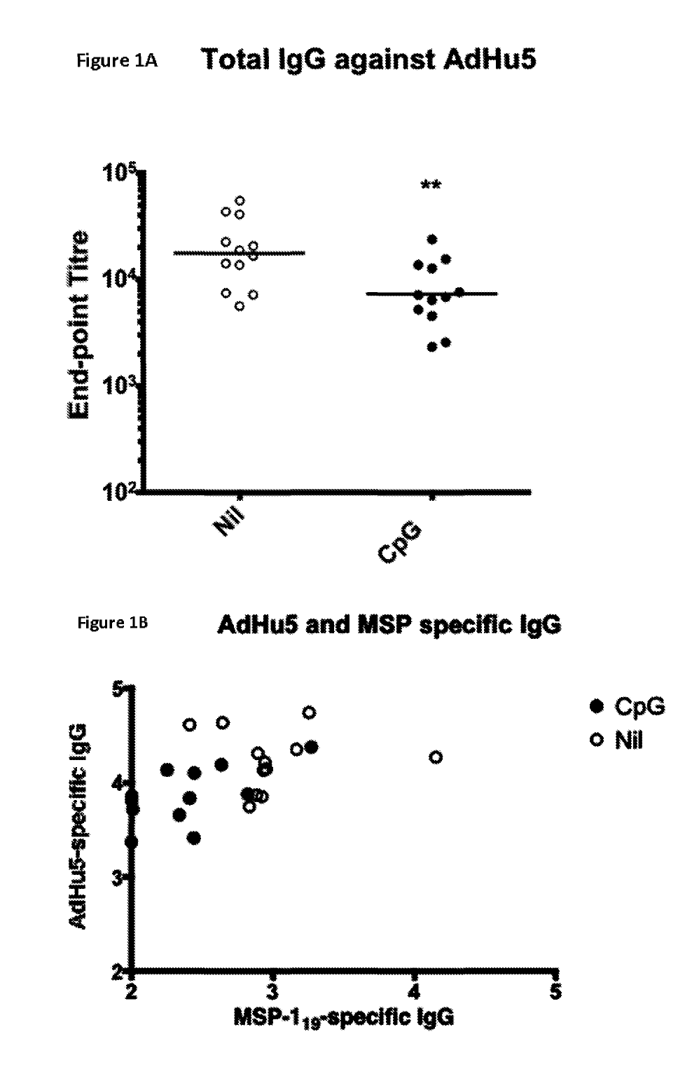

The percentage of peripheral blood and splenic CD8⁺ T cells expressing IFNγ, TNFα and IL-2 in response to 5 h stimulation with 5 μg/mL peptides 90 and 91 was assessed by intracellular cytokine staining as previously described [Goodman A L, Epp C, Moss D, et al. Infect Immun. 2010 Aug. 16.]. Surface staining was with anti-CD8a PerCP-Cy5.5 and anti-CD4 Pacific Blue while intracellular staining was with anti-IFNγ APC, anti-TNFα FITC and anti-IL-2 PE (all supplied by eBioscience, UK). Cytokine production frequency in peptide-unstimulated control wells (which was typically <0.1%) was subtracted from the result in peptide-stimulated wells prior to further analysis. The gating strategy is illustrated in supplementary FIG. 1.

Antibody Responses—Total IgG, Isotypes and Avidity

Total IgG and isotype ELISA were carried out as previously described using bacterially expressed GST-tagged PfMSP1₁₉ (Wellcome/FVO allele) as the coating antigen [Goodman A L, Epp C, Moss D, et al. Infect Immun. 2010 Aug. 16.].

Antibody avidity was assessed by sodium thiocyanate (NaSCN)-displacement ELISA [Ross T M, Xu Y, Bright R A, Robinson H L. Nat Immunol 2000 August; 1(2):127-31.]. Using previously measured total IgG ELISA titers, sera were individually diluted to a level calculated to give a titer of 1:300 and plated at 50 μl/well in 16 wells of a 96 well plate. Following incubation and washing, an ascending concentration of the chaotropic agent NaSCN was added down the plate (0 to 7M NaSCN). Plates were incubated for 15 min at room temperature before washing and development as for total IgG. The intercept of the OD₄₀₅ curve for each sample with the line of 50% reduction of the OD₄₀₅ in the NaSCN-free well for each sample (ie. the concentration of NaSCN required to reduce the OD₄₀₅ to 50% of that without NaSCN) was used as a measure of avidity.

Statistical Analysis

Statistical analysis was carried out using Prism 5 software (GraphPad, La Jolla, Calif., USA). All ELISA titers were log₁₀ transformed prior to analysis. Graphs indicate sample arithmetic means; error bars where present indicate 95% confidence intervals for the population arithmetic mean. One-way ANOVA was used for comparing normally distributed data with Bonferroni's multiple comparison post-test for comparison of specific groups; Kruskal-Wallis tests were used for comparison of non-normally distributed data with Dunn's multiple comparison post-test for comparison of specific groups. Two-way ANOVA was used for comparison of groups differing in two factors. Two-way repeat measures ANOVA was used for comparison of responses measured for different groups at different time points, after the exclusion of the small number of mice for which replicate data were not available at all time points. P<0.05 was taken to be statistically significant throughout.

### Example 2

### Several Adjuvants Fail to Enhance Immune Responses from Vectors

i) TLR Agonists

ii) Carbopol

i) TLR Agonists

The effect of TLR 3 stimulation on immune responses to a human adenoviral vector (AdHu5) vaccine was assessed. PfM115 is a P. falciparum antigen construct based on merozoite surface protein 1 (MSP1). To determine the immune modulating effects of TLR 9 stimulation, C57BL/6 mice were immunised once with AdHu5 PfM115 mixed with PBS or with the TLR 9 agonist, CpG ODN 1826. Note that this AdHu5 vector has deletions in both the E1 region and the E3 region ensuring replication incompetence in almost all mammalian cells and increasing the size of the insert that can be used to >5 kb. There was no correlation between total IgG against the backbone AdHu5 and total IgG against the antigen PfMSP-1₁₉ as measured by ELISA whether using data generated from CpG-treated mice only (P=0.11, Pearson's correlation) or non-treated mice only (P=0.97), though there was a trend towards a weak correlation when both groups were combined in the analysis (P=0.07, R2=0.16) (FIG. 1b).

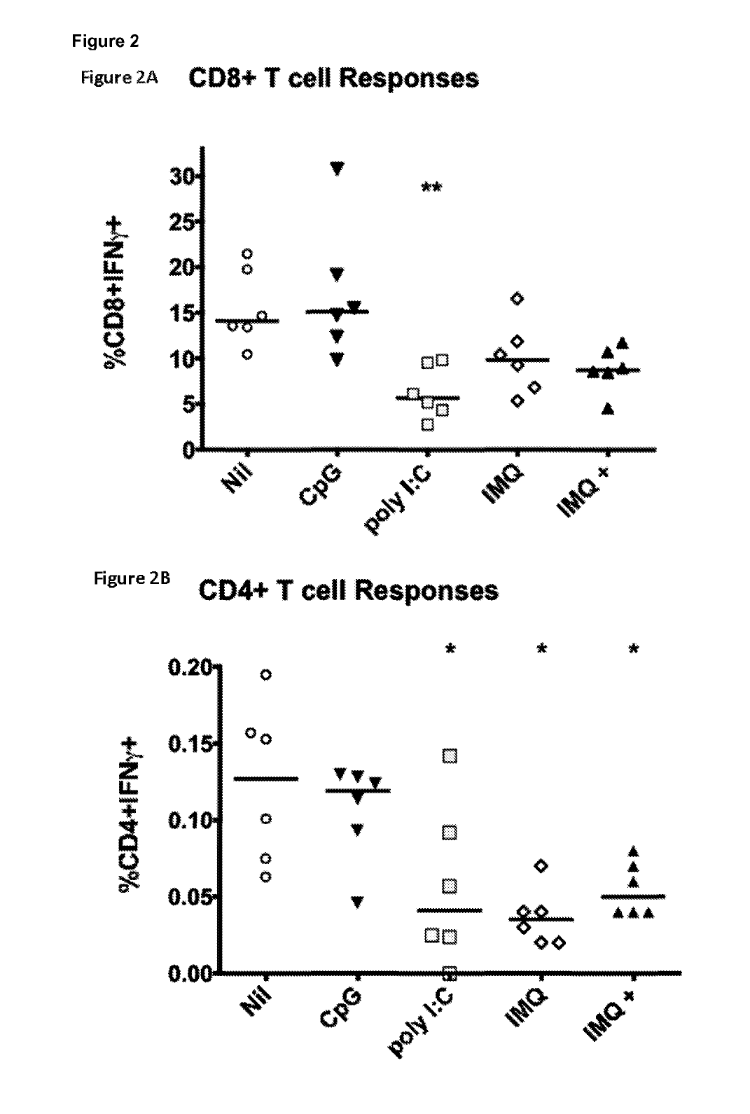

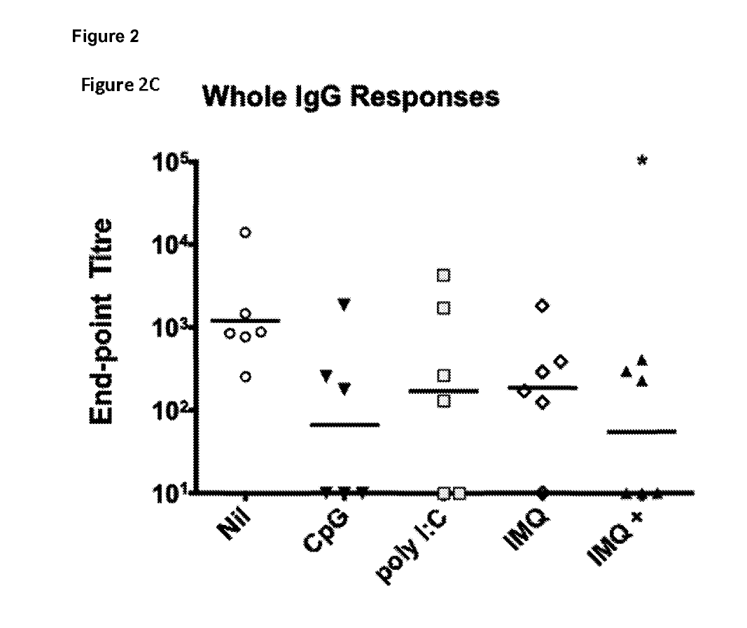

The same vectored vaccine was also combined with poly I:C (TLR 3 agonist) and Imiquimod (TLR 7 agonist). Poly (1:0) had a significant suppressive effect on CD8+ and CD4+ T cell responses (FIG. 2a,b) and there was a trend towards reduced antibody responses to PfMSP1₁₉ when assessed by ELISA (FIG. 2c). The application of a TLR 7 agonist, topical imiquimod, at the same time as the AdHu5 PfM115 vaccine (IMQ) or one hour later (IMQ+) led to a suppression of cellular and humoral immune responses as shown in FIG. 2. Only the difference in CD4+ responses was statistically significant (P<0.05, one-way ANOVA with Dunnett's correction). The delayed group (IMQ+) was included in this experiment in an attempt to prevent direct action of imiquimod on the AdHu5 vector backbone. It was hypothesised that a time interval between administration of vaccine and TLR agonist might prevent a direct interaction.

ii) Carbopol

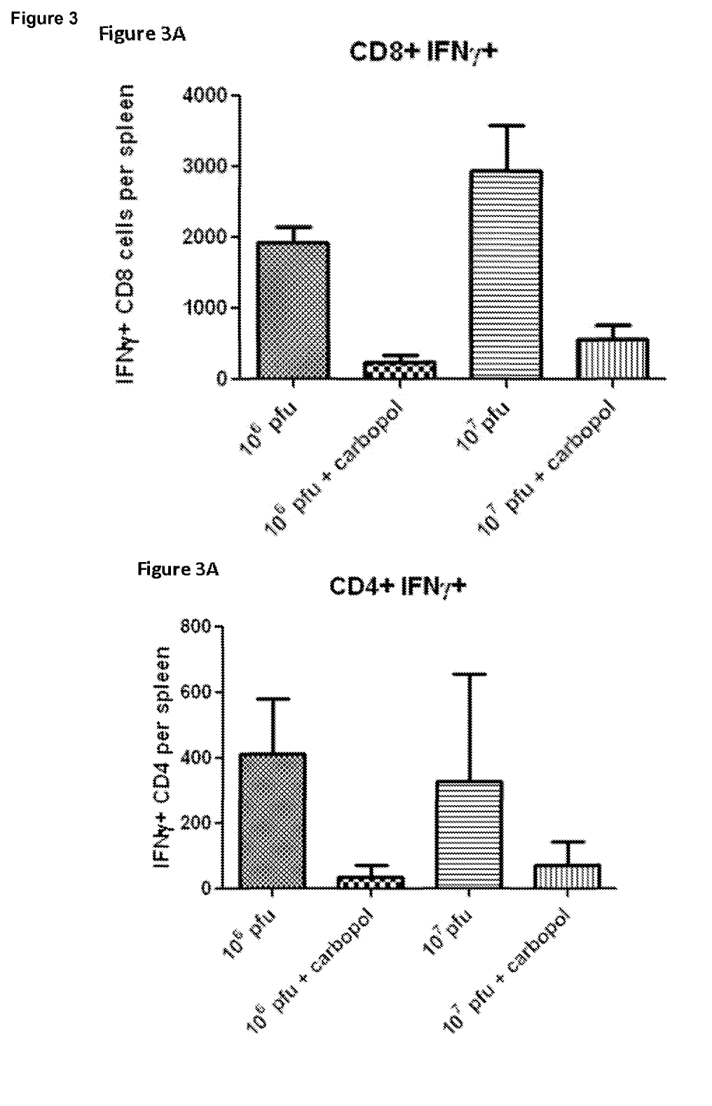

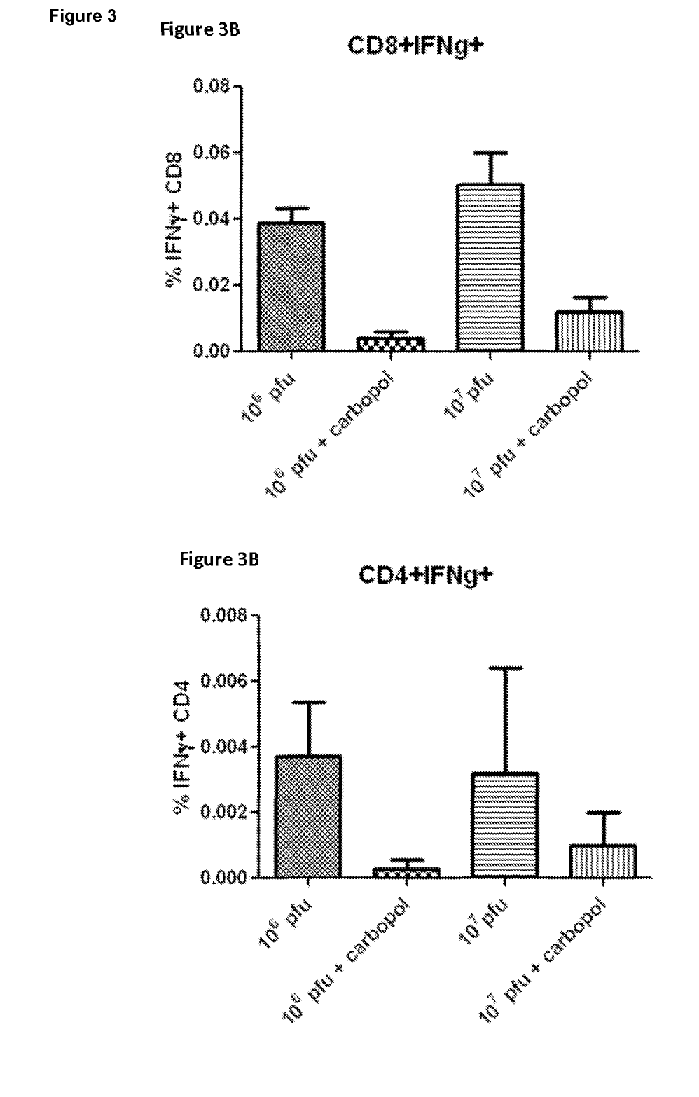

The immunogenicity of MVA vaccine expressing P. yoelii MSP1₄₂ was assessed with and without co-formulation with Carbopol adjuvant. A reduction was seen in both MSP1-specific CD8+ and CD4+ T cell responses when the MVA vaccine was formulated with Carbopol 3 adjuvant (FIG. 3). No PyMSP1₁₉-specific IgG responses were detectable by ELISA in the serum of any of the immunised mice (data not shown).

### Example 3

### Combining ISCOM Matrix Adjuvant with an Adenoviral Vector and Protein in a Three-Component Malaria Vaccine Induces Stronger CD4 T Cell Responses as Well as Significantly Higher Antibody Titres

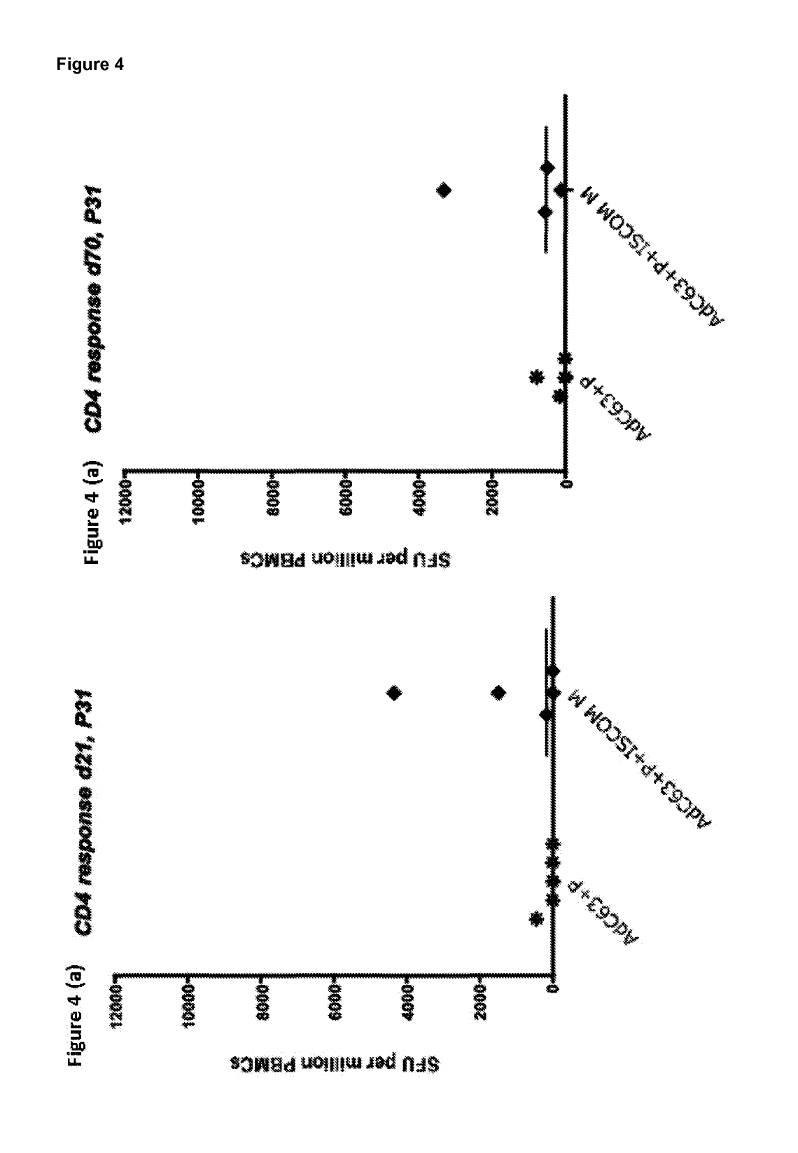

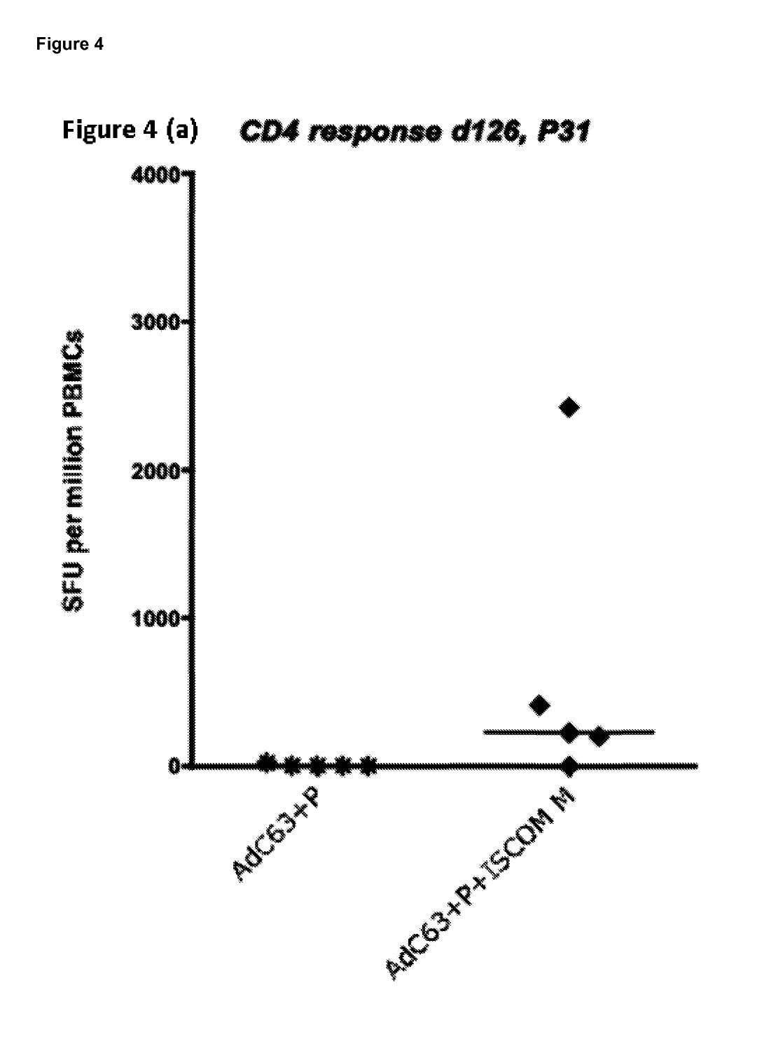

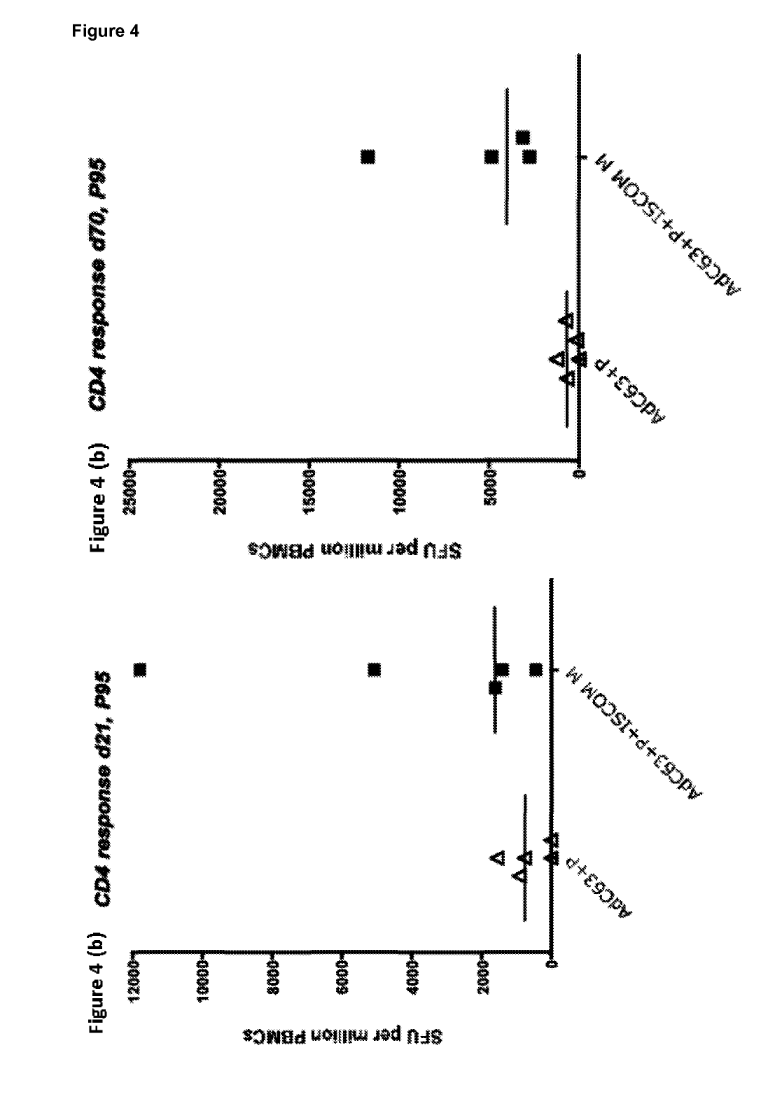

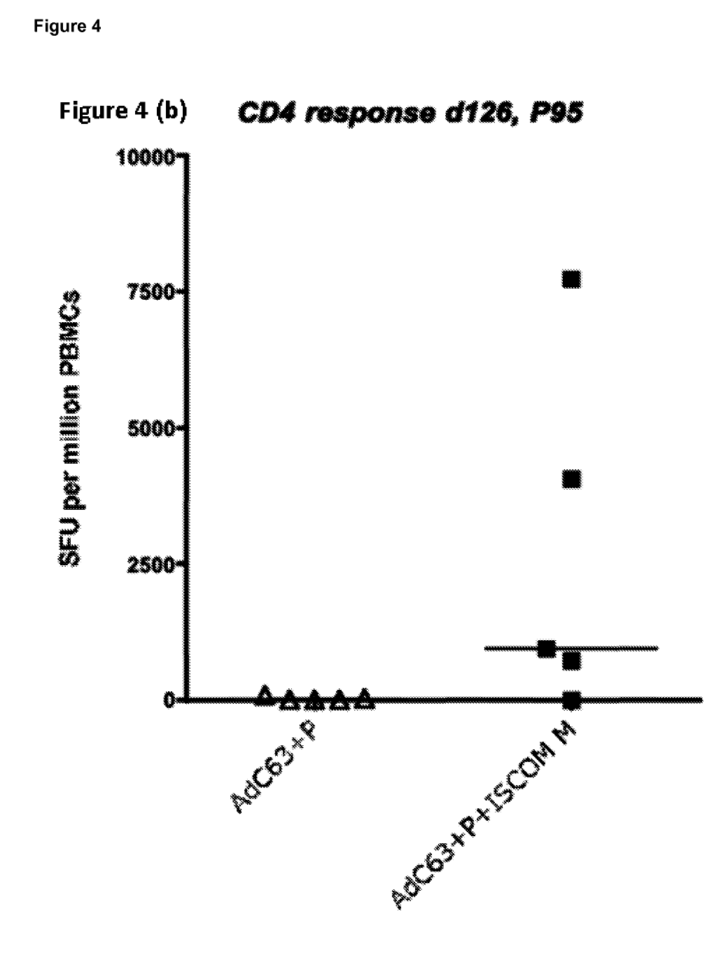

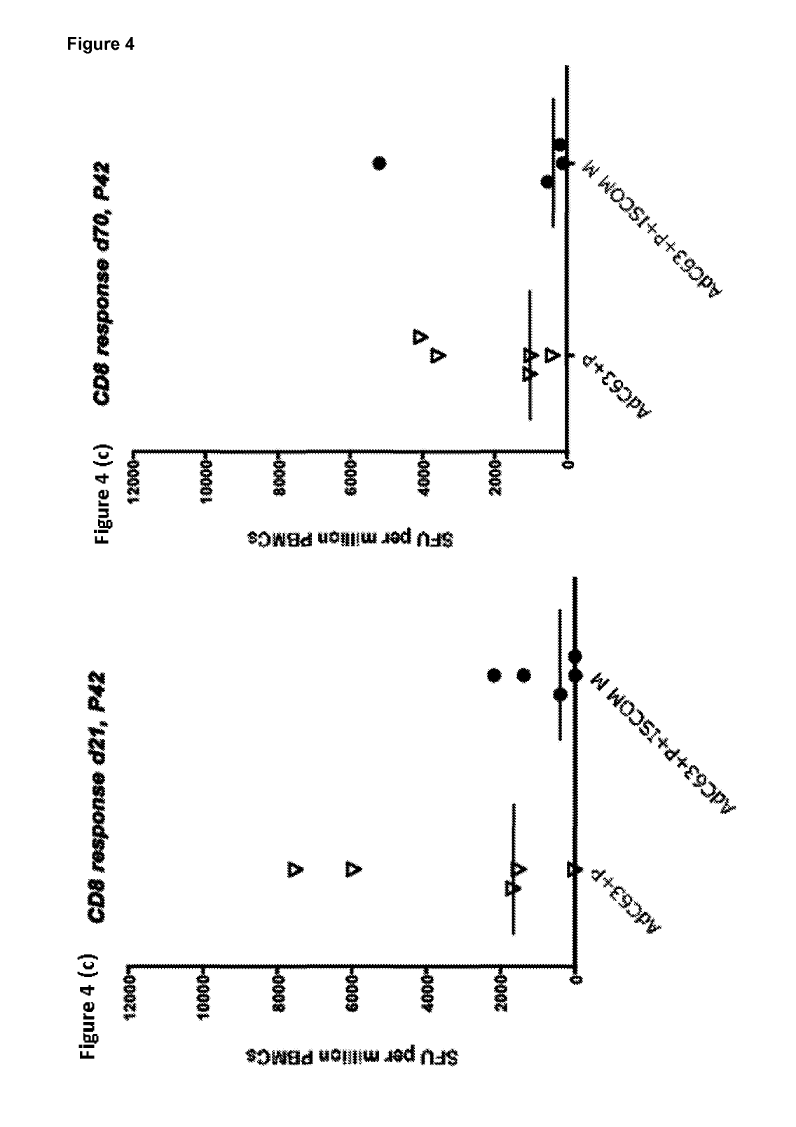

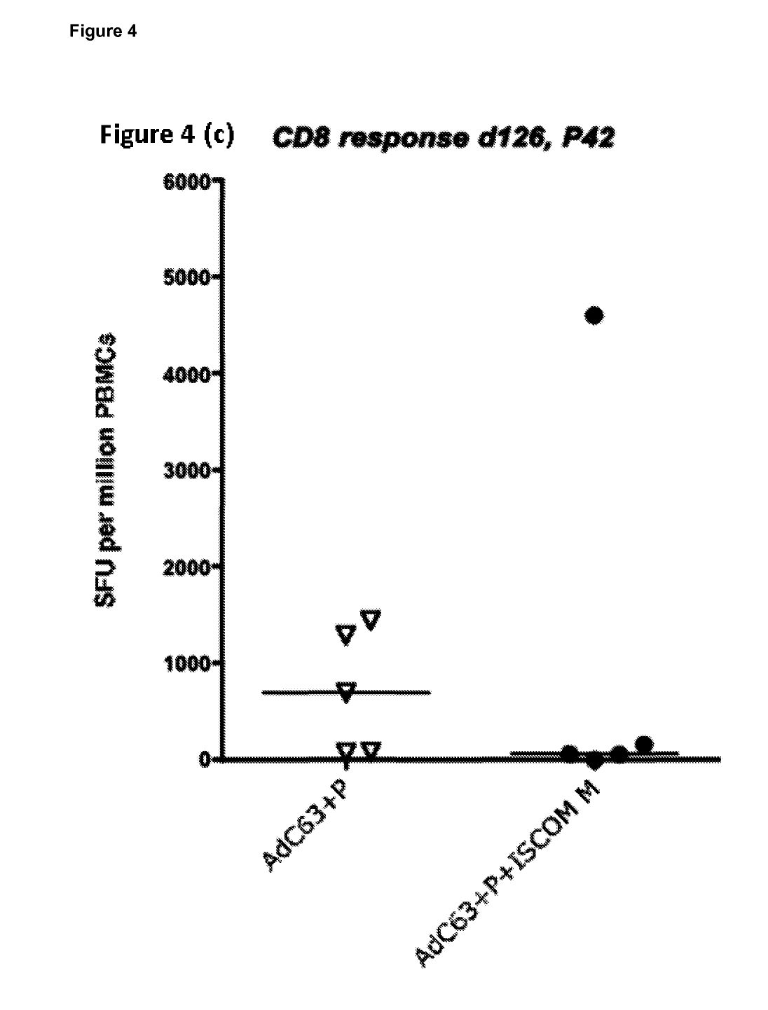

We tested the effect of the ISCOM Matrix M adjuvant on the cellular and humoral immune responses induced by a vaccine containing simian viral vector encoding apical membrane antigen-1 (AMA1) gene mixed with AMA1 protein. To this effect, we combined AdCh63-AMA1 (5×108 v.p.), AMA1 protein (10 μg) and ISCOM Matrix M (12 μg) and we immunised BALB/c mice with the adjuvanted or non-adjuvanted vaccine (n=5 per group) three times, with 8 week intervals between homologous vaccinations. Two weeks after each vaccination (weeks 2, 10 and 18), we assayed AMA1-specific T cell responses in whole blood using whole-blood ELISpot. We also tested for AMA1-specific antibody titres at weeks 2, 7 and 10. 6 Following vaccination, we noted an increase in the proportion of IFN-γ producing peripheral blood CD4 T cells in the presence of the ISCOM Matrix adjuvant at all three time-points. Conversely, responses to a CD8 T cell restricted peptide were (non-significantly) reduced at all time points (FIG. 4).

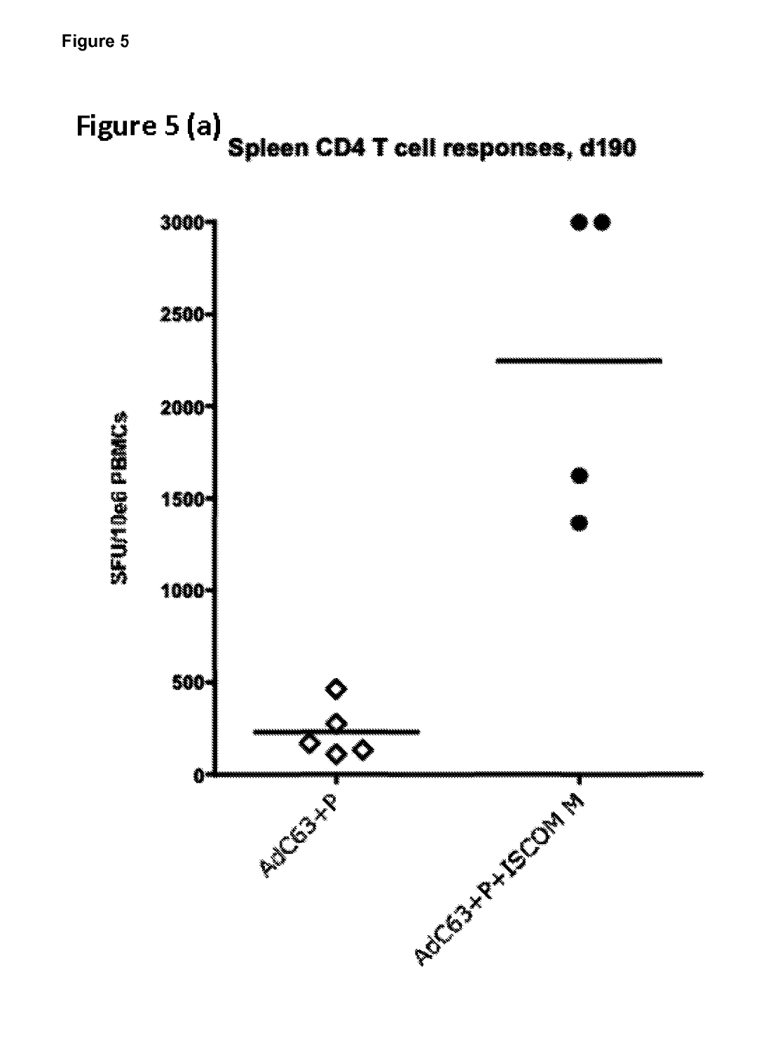

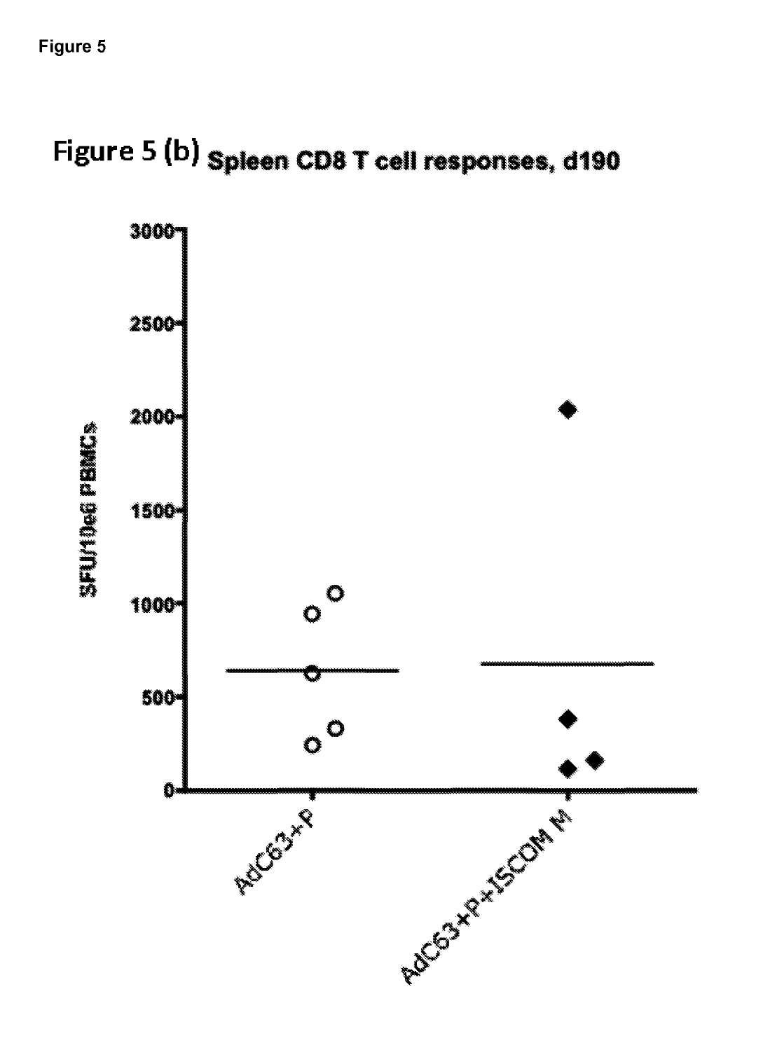

At the final time-point, six months after the first vaccination (week 24) the vaccinated mice were sacrificed and spleens analysed for antigen specific IFN-γ production by CD4 and CD8 T cells. The group receiving the adjuvanted vaccine showed a significantly higher CD4 splenocyte response compared to the non-adjuvanted vaccine group (p=0.0013, 2-tailed t-test). In parallel with the observations of T cell responses in peripheral blood, the antigen-specific CD8 T cell production of IFN-γ in the spleens was similar in the two groups (FIG. 5).

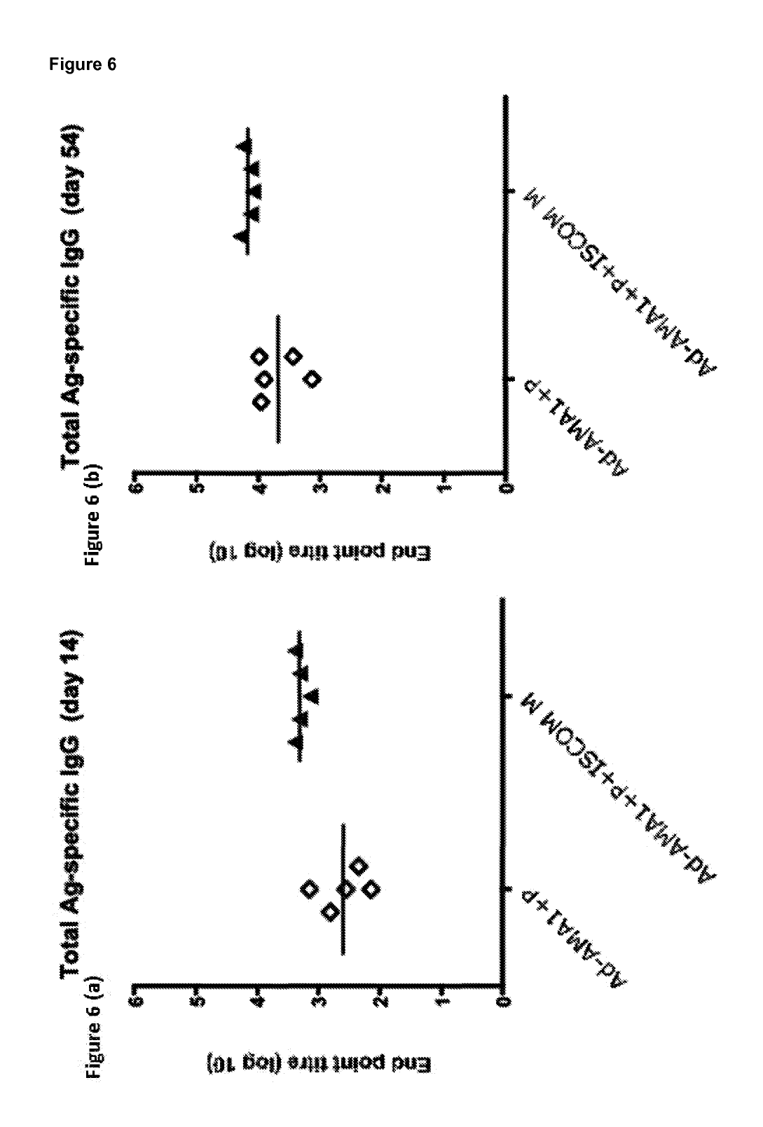

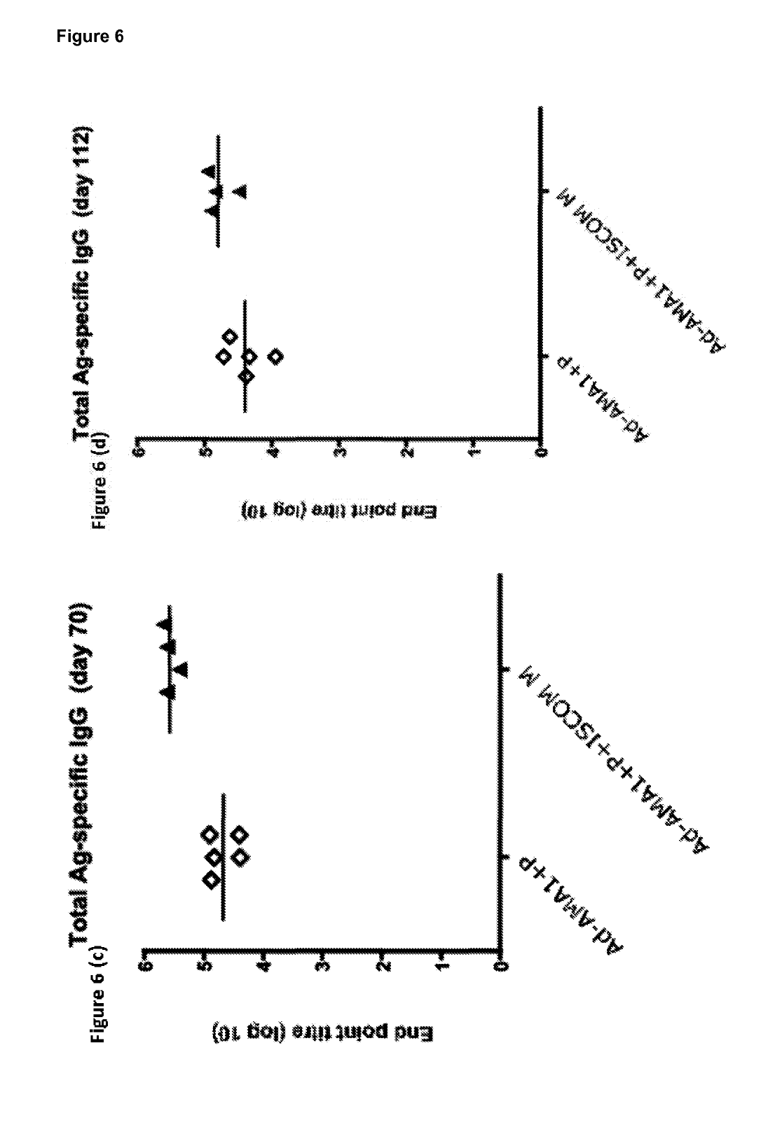

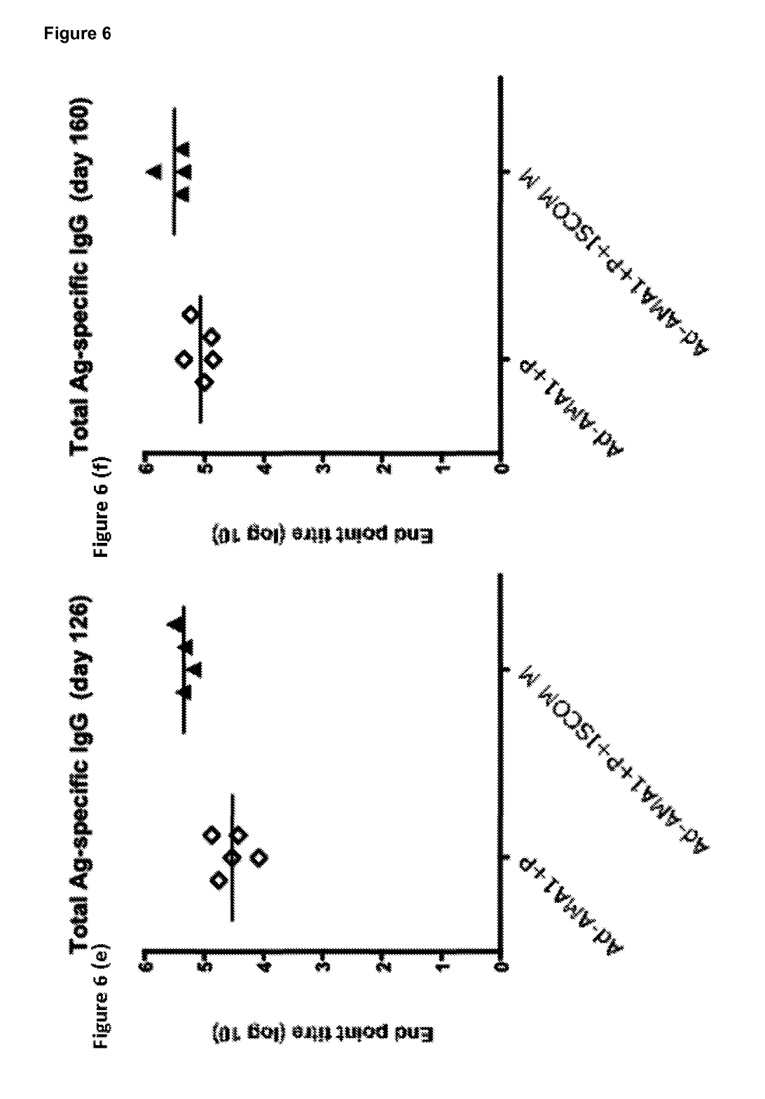

Analysis of antigen specific total IgG antibody responses also showed higher titres following immunisation with the ISCOM Matrix M adjuvanted vaccine at all time points assessed. On day 70 (two weeks after the third vaccination) antigen specific antibody titres in the group receiving the adjuvanted vaccine were significantly higher (p=0.0004, unpaired, 2-tailed t-test) than in the group receiving vaccine without the adjuvant (FIG. 6).

### Example 4

### Combining ISCOM Matrix Adjuvant or Oil-in-Water Emulsion with Adenoviral Vectored Malaria Vaccine Enhances Antigen Specific CD4 T Cell Responses

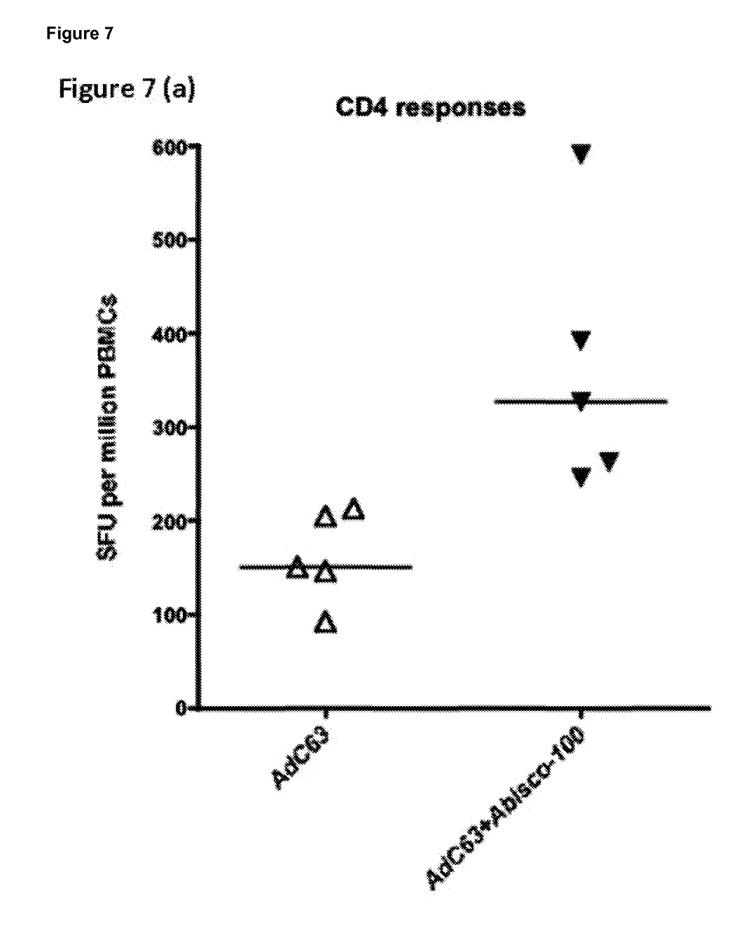

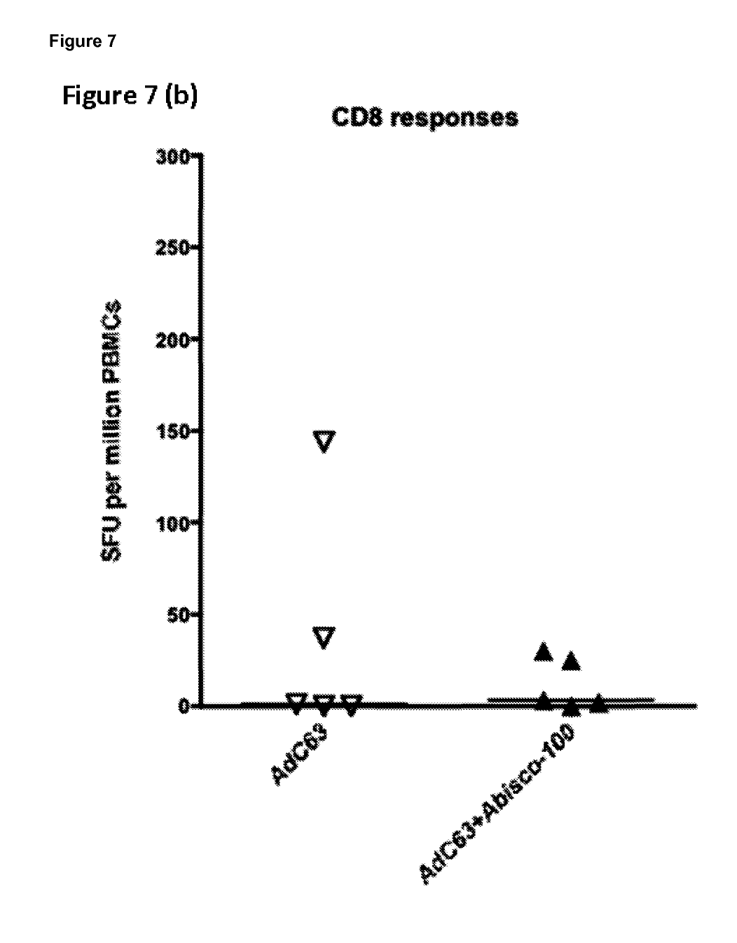

In a separate experiment, we tested the short-term effect of adding ISCOM Matrix M to a vaccine containing simian viral vector encoding apical membrane antigen-1 (AMA1). BALB/c mice were immunized once with the adjuvanted or non-adjuvanted vectored vaccine AdCh63-AMA1 (5×108 viral particles, n=5 per group). Two weeks after the vaccination we assayed AMA1-specific IFN-γ CD4 and CD8 T cell responses in the spleen using ELISpot. We found a significant increase in the proportion of CD4+ IFN-γ producing splenocytes in the group that received the adjuvanted vaccine (p<0.01, FIG. 7a). Responses to a CD8 T cell restricted peptide were comparable between the two vaccines (FIG. 7b).

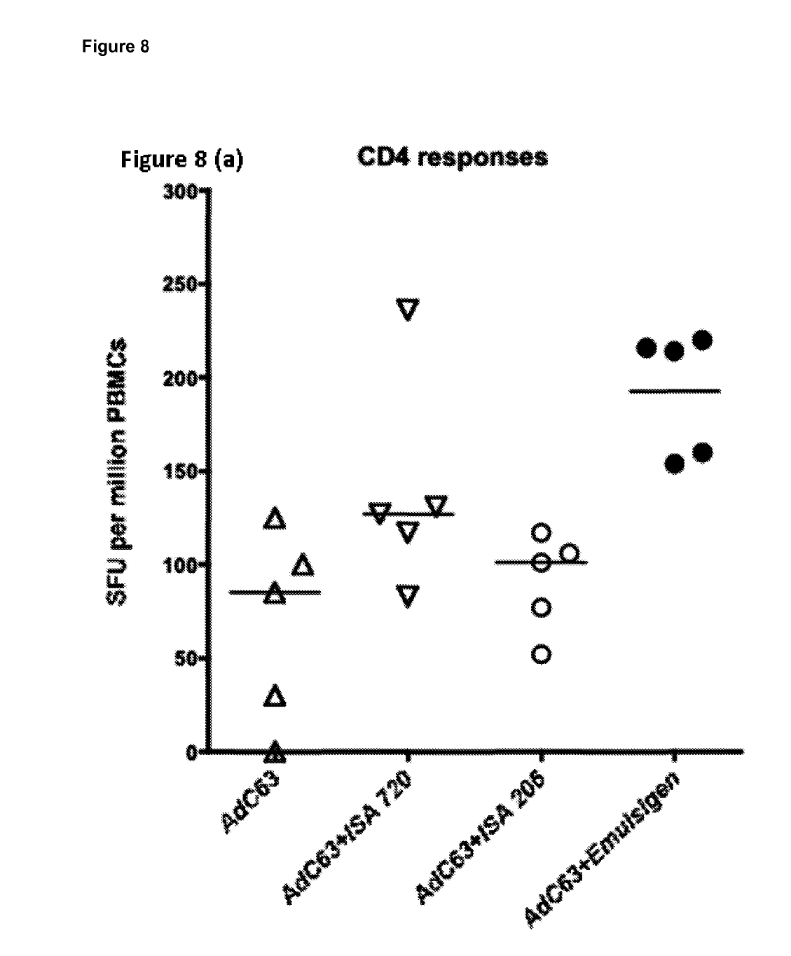

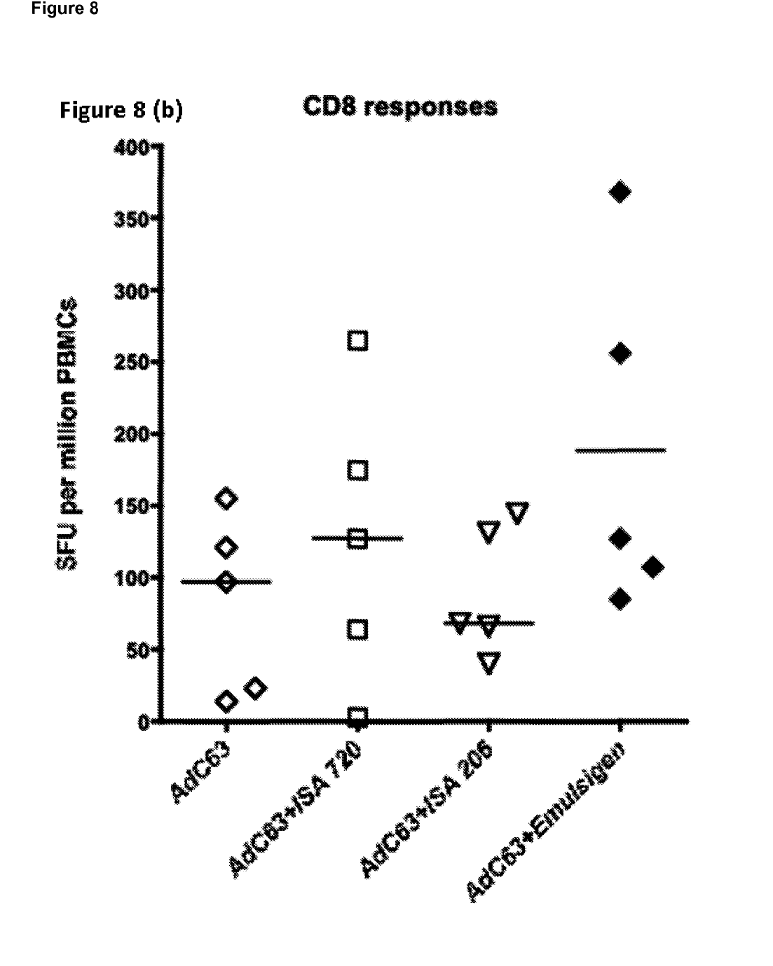

Vaccination with AdCh63-AMA1 vaccine adjuvanted with oil and water emulsions also resulted in an overall increase in IFN-γ responses by CD4 T cells with all of the tested emulsions and reached statistical significance with Emulsigen, an oil-in-water emulsion (p<0.05). The IFN-γ production by CD8 T cells was again comparable to the non-adjuvanted vaccine with a higher trend in the group vaccinated with AdCh63-AMA1 combined with Emulsigen (FIG. 8).

### Example 5

### Addition of the Adjuvant ISCOM Matrix Increases the Efficacy of the Ad-ME.TRAP Malaria Vaccine

We assessed the effect of the adjuvant ISCOM Matrix on the cytokine CD8 responses induced by Ad-ME.TRAP immunisation, using an AdCh63 vector encoding the ME.TRAP antigen. Note that this Ad vector has deletions in both the E1 region and the E3 region ensuring replication incompetence in almost all mammalian cells and increasing the size of the insert that can be used to >5 kb. The ME.TRAP transgene consists of the TRAP sequence of P. falciparum, attached to a multi-epitope (ME) string that expresses Pb9, an H2K(d)-restricted epitope (SYIPSAEKI) that is immunodominant in BALB/c mice. To this effect, two groups of 5 BALB/c mice each were immunized bilaterally, intradermally into the ear pinnae with Ad-ME.TRAP at 5×10⁹ vp/mouse in 25 μl volume per ear. A control group received the vaccine in PBS.

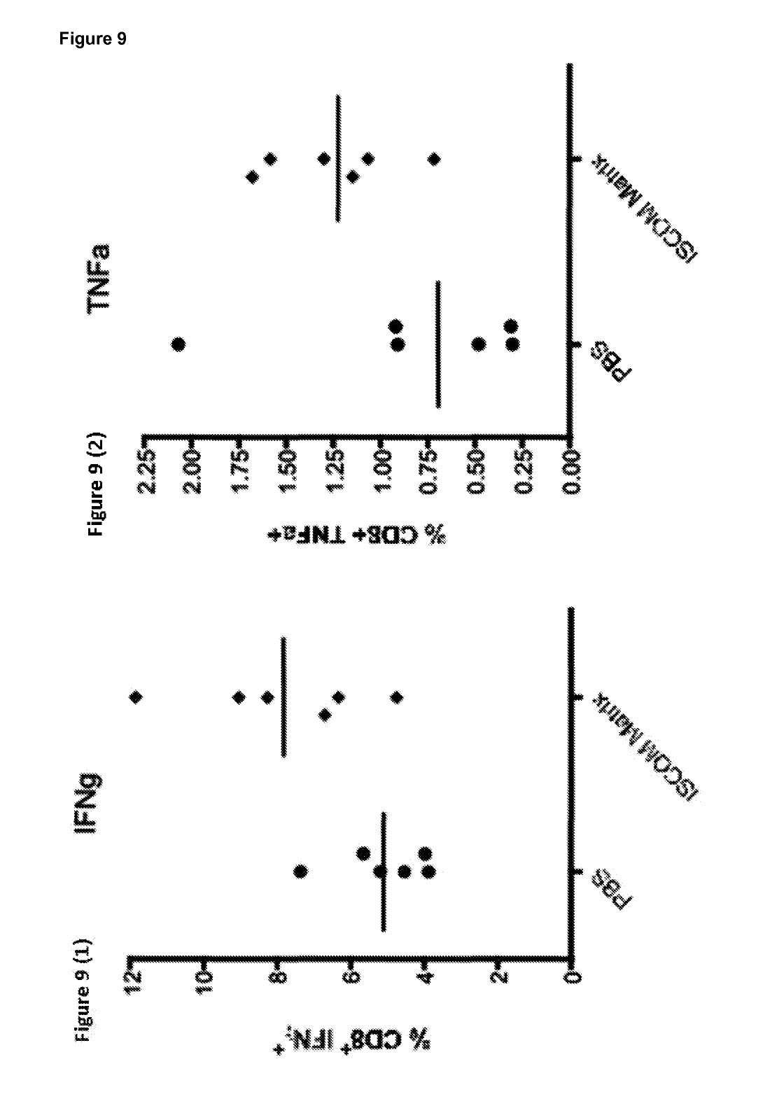

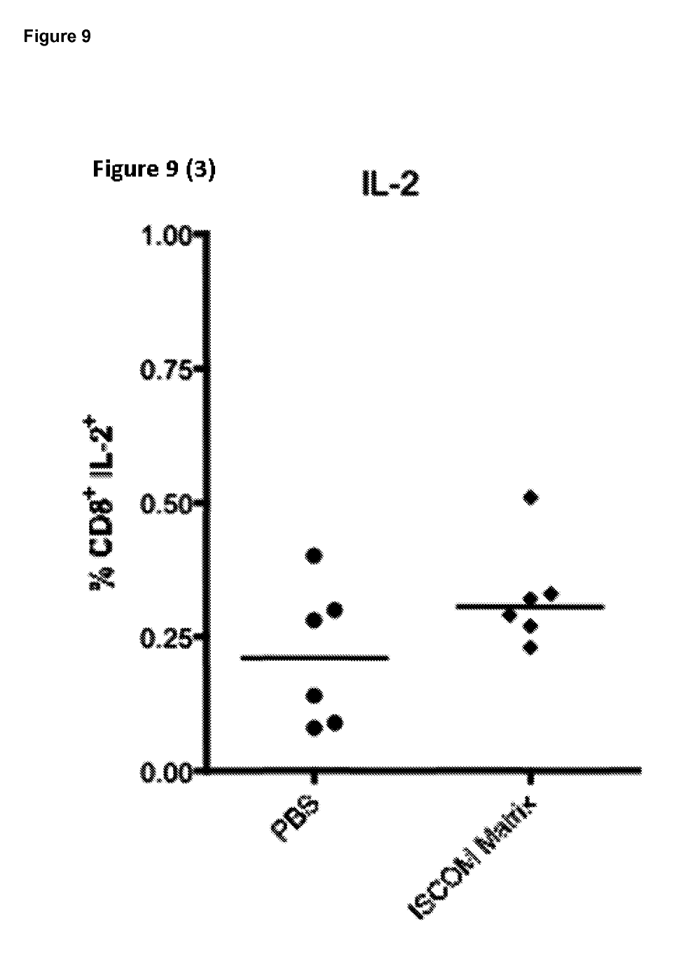

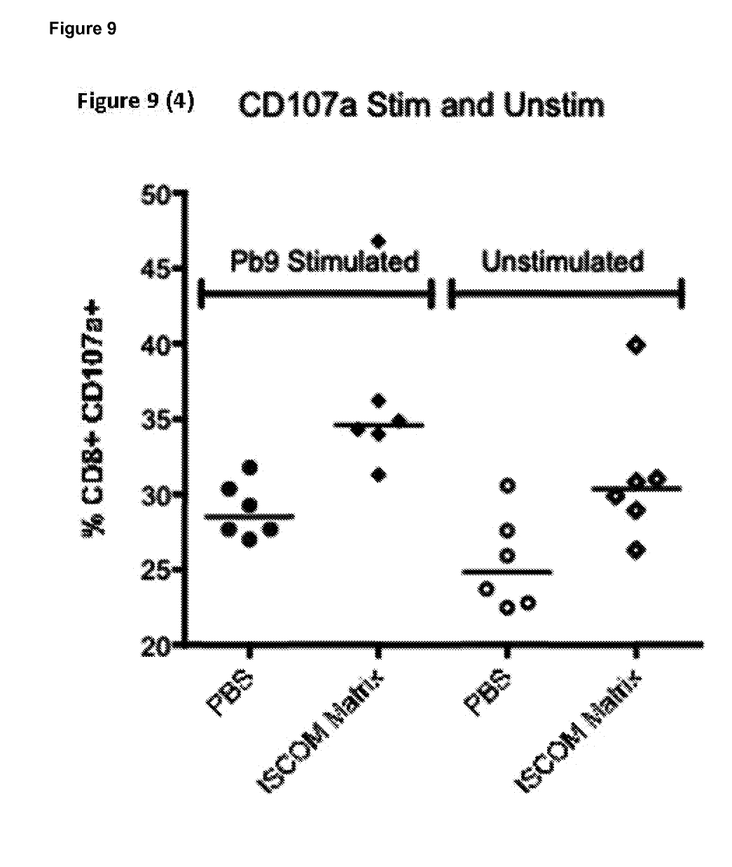

Following the administration of Ad-ME.TRAP adjuvanted with the ISCOM Matrix, we noted a non-significant increase in the frequencies of antigen-specific CD8 T-cells producing IFN-γ, TNF-α and IL-2 in blood, as well as in the expression of the degranulation marker CD107a, both unspecific and peptide stimulated, indicating a lack of significant differences in the production of the assayed cytokines by CD8 T cells between the non-adjuvanted and adjuvanted vaccine (FIG. 9).

Two weeks following the immunisation, the mice were challenged with malaria by an intravenous delivery of 1,000 sporozoites per mouse. The progress of the infection was monitored by counting parasite numbers on blood smears starting from day 5 post-challenge. Vaccine efficacy was measured as a delay in reaching detectable blood parasitaemia and we found that addition of ISCOM Matrix resulted in a 2-day delay in the development of parasitaemia in the blood (FIG. 9).

### Example 6

### Addition of a Water-in-Oil Emulsion to Malaria Antigen Protein and Viral Vectored Vaccine Leads to Increased Antibody and T Cell Responses and can Reduce Number of Vaccinations Required to Reach the Same Immunogenicity

Immunogenicity of Two Component Regimes

The experimental design provided replicate groups receiving AdCh63-MVA (A-M) and AdCh63-protein (A-P) sequential regimes at 57 day and 97 day intervals. These data were analysed by two-way ANOVA, demonstrating that antibody responses 14 days post boost were greater with the A-P regime than the A-M regime (FIG. 1A) (P<0.0001), and greater with the 97 day interval than the 57 day interval (P=0.0006). The antibody response induced by protein-protein (P-P) vaccination was markedly variable with three mice mounting high responses comparable to those receiving A-P immunization, and three very weakly responding mice (FIG. 1A-B). There was no significant difference between median antibody responses following protein-protein, adenovirus-MVA and adenovirus-protein regimes after a 57 day dose interval (P=0.37 by Kruskal Wallis test), but there was a clear increase in the variance of the response after two shot protein regimes compared to viral-vector containing regimes.

In contrast with the antibody results, greater percentages of IFNγ+CD8+ T cells were detected by ICS 14 days after A-M immunization than A-P, and the 57 day dose interval was superior (P<0.0001 for both comparisons). Clear boosting of CD8+ T cell responses by MVA was evident at both dose intervals. As expected, given the lack of the CD8+ T cell epitope in the MSP119 protein sequence in BALB/c mice, CD8+ T cell responses were not detectable following P-P vaccination. Additional experiments in C57BL/6 mice (in which a CD8+ T cell epitope is present in the MSP119 protein) confirmed that, in contrast to the A-M regime, P-P vaccination did not induce a CD8+ T cell response detectable by IFNγ splenic ELISPOT or peripheral blood ICS, and that CD8+ T cell responses were unaltered by A-P immunization as compared to adenovirus priming alone. CD8+ T cell responses after A-P immunization of either mouse strain thus presumably represent the contracting or effector memory CD8+ T cell response induced by the adenovirus.

Immunogenicity of Three-Component Sequential Regimes

We subsequently compared the immunogenicity of three-component sequential adenovirus-MVA-protein (A-M-P) and adenovirus-protein-MVA (A-P-M) regimes to two-component regimes (FIGS. 2 and 3). The kinetics of the responses induced by these regimes were markedly different. We found that addition of protein to adenovirus-MVA (A-M-P) was able to boost antibody but not CD8+ T cell responses (again as would be predicted due to lack of the T cell epitope in this protein), while addition of MVA to adenovirus-protein (A-P-M) boosted CD8+ T cell responses but not antibody titer. Total IgG responses to A-M-P and A-P-M were significantly higher than those to A-M (P<0.05 by ANOVA with Bonferroni post-test), with no significant differences between the responses to A-M-P, A-P-M and A-P (P>0.05). There were no statistically significant differences in CD8+ T cell responses between A-M-P, A-P-M and A-M regimes (P>0.05 by ANOVA with Bonferroni post-test). In general, any two- or three-component regime including AdCh63 and MVA induced maximal CD8+ T cell responses as measured in the blood. Conversely, maximal IgG responses were elicited by any regime including AdCh63 and protein.

Regimes Mixing Viral-Vectored and Protein-Adjuvant Vaccines

We continued to investigate whether the advantages of three-component regimes could be achieved in a simplified two-stage regime, by mixing protein and adjuvant with one or both viral vector components. We found that there was no significant difference by Kruskal-Wallis test between the three-immunization regimes and a two-immunization regime mixing protein and Montanide ISA720 with both adenovirus prime and MVA boost. Interestingly, there was a small but statistically significant increase in CD8+ T cell responses and decrease in antibody responses with the (A+P)-M regime relative to A-P-M (P<0.05, ANOVA with Dunn's multiple comparison post-test). Antibody responses tended to be highest with the three component regimes, or when protein-adjuvant was co-administered with both viral vectors. Interestingly, in C57BL/6 mice, (A+P) priming induced modestly but significantly higher CD8+ T cell responses than adenovirus alone (P=0.04, Mann-Whitney test).

Thus a simplified two-shot immunization regime appears highly immunogenic and mixing of the viral vectors with protein and adjuvant did not appear to affect vector potency, a result which may encourage development of further strategies combining vectors with protein and adjuvant, including homologous vector-protein prime-boost immunization regimes.

Longevity of Responses

Serum antibody and splenic T cell responses were assayed by ELISA and IFNγ ELISPOT 138 days after final vaccination for selected groups of. Antibody responses to A-M-P and A-P-M remained significantly higher than those for A-M (P<0.05 for both comparisons by Kruskal-Wallis test with Dunn's multiple comparison post-test), while CD8+ T cell responses following A-M-P and A-M remained greater than those for A-P (P<0.01 and P<0.05 respectively by the same method). There was a mean drop of 0.4 log units in ELISA titer between 14 and 138 days after final vaccination, with no significant difference in this rate of decline between groups (FIG. 5C, P=0.37 by Kruskal Wallis test). Thus, as was the case with early post-vaccination responses, maximal long-lived IgG responses were detected with any regime including AdCh63 and protein, while any regime including AdCh63 and MVA induced maximal long-lived CD8+ T cell responses in the spleen.

Immunization Routes and Doses

We also compared the antibody and CD8+ T cell responses of six mice receiving the A-M-P regime entirely intramuscularly versus six mice receiving the viral-vector components intradermally (i.d.). There was no significant difference by t-test between the two groups' log ELISA titer (P=0.26) or % IFNγ+CD8+ T cells (P=0.20) 14 days after final vaccination, nor was a difference found between groups for either ELISA or CD8+ T cell responses by repeat measures ANOVA taking into account all time points up to 14 days after final vaccination.

In parallel, we had conducted the same experiments at lower vaccine doses (108 vp AdCh63, 106 pfu MVA, and 5 μg protein at 8 week intervals) in BALB/c mice, in case a ‘ceiling’ or maximum dose-response effect prevented us observing differences between the higher dose regimes used in the previous experiments. Importantly, similar patterns to those previously observed were apparent from the lower dose experiment. As expected all antibody and T cell responses were substantially weaker when using lower vaccine doses. Responses to protein-protein vaccination were markedly more variable than responses to adenovirus-containing regimes. At these lower doses, addition of protein did not enhance the antibody immunogenicity of viral vector regimes, with no significant differences in ELISA titers following A-M, A-P, A-M-P or A-P-M vaccination. T cell responses were again substantially higher in the A-M, A-M-P and A-P-M groups than in the A-P group. As before, the (A+P)-M, A-(M+P) and (A+P)-(M+P) two-stage regimes mixing viral and protein vaccines produced results similar to three-stage vaccination, with a trend towards higher antibody but lower CD8+ T cell responses in the group receiving (A+P)-(M+P). Thus despite the clearly sub-maximal responses achieved in these animals (in particular with the protein only vaccination), regimes incorporating adenovirus and MVA again appeared to result in more consistent combined antibody and CD8+ T cell responses to the antigen.

Antibody Isotypes

To further characterize the immune responses to the various vaccine modalities, we performed IgG isotype ELISAs. It was not possible to measure isotype-specific titers for the three P-P immunized mice with low total IgG ELISA titers. Bearing in mind this limitation, viral-vector-containing regimes induced a significantly greater ratio of IgG2a to IgG1 than was present in the high-total-titer P-P immunized mice, and that the IgG2a/IgG1 ratio was higher for all groups 137 days rather than 14 days after the final vaccination, corresponding to better maintenance of the titer of IgG2a than IgG1 over time (P<0.001 for both comparisons by repeated measures two-way ANOVA with Bonferroni's post test). There was no interaction of time and regime (i.e. no inter-regime differences in the rate of change of the IgG isotype balance over time).

Antibody Avidity

We continued to investigate the responses to the various regimes by measuring antibody avidity using NaSCN antibody-displacement ELISA for selected groups and time points. Among mice receiving A-M and A-P regimes, we observed that mice receiving A-M had higher antibody avidity 14 days post-boost than those receiving A-P, without any significant difference between 57 day and 97 day dose interval (P=0.024 for regime comparison, P=0.33 for comparison dose interval by two-way ANOVA). Looking more widely at mice receiving A-M-P, A-P-M, A-M, A-P and P-P regimes, it was apparent that there was a trend for higher avidity in mice receiving any regime including both viral vectors (A and M) than in those receiving only A-P or P-P. When analyzed by two-way repeat measures ANOVA, this trend did not reach statistical significance (P=0.32) without pooling of replicate groups (described above for A-P and A-M), though there was a significant increase in avidity over time after final vaccination across all groups (P<0.0001). There was no correlation between total IgG ELISA titer and avidity, either when data from all time points were combined (FIG. 8C, r2=0.00, P=1.00 by linear regression) or where each time point was analyzed separately (data not shown). Thus antibody avidity and total IgG ELISA titer appear to vary independently, and avidity appears to rise over time post-boost and with MVA-containing regimes.

Splenic Antibody Secreting Cells

At the conclusion of the experiment (138 days after final vaccination), mice were sacrificed and antigen-specific antibody secreting cells (ASCs) in the spleens of four mice from each group were counted using an ex-vivo assay without a proliferative culture step. This non-cultured assay at such a late time point would be expected to detect the presence of long-lived plasma cells. Log transformed ASC counts differed between groups (P=0.04 by Kruskal Wallis test) with a trend towards the highest ASC counts in groups receiving three component regimes (A-M-P and A-P-M), and the lowest ASC count in mice receiving A-M. Differences between individual groups however did not reach statistical significance after correcting for multiple comparisons using Dunn's post test. There was a reasonable linear correlation between log transformed ASC counts and log transformed total IgG ELISA titers, present using either peak ELISA titer 14 days after final vaccination (data not shown), or late ELISA titer 138 days after final vaccination (for late time point, r2=0.39, P=0.004).

T Cell Functionality

The ICS antibody panel stained for IFNγ, TNFα and IL-2, thus allowing quantification of single, double and triple cytokine positive antigen-specific CD8+ T cells in the blood at the time points assayed. Given the lack of a CD8+ T cell epitope in the protein vaccine, the A-P group can be viewed as an unboosted control. The majority of T cells positive for a single cytokine were IFNγ+. Those positive for a second cytokine were mostly IFNγ+ TNFα+, in accordance with previous observations using viral-vector P. yoelii MSP142 vaccines. Few cells expressing IL-2 were observed with any regime. Comparing the various three-stage and two-stage regimes including both adenovirus and MVA, although there was some variation between regimes in the proportion of double cytokine positive cells relative to single positive cells, there was no difference in the proportion of double cytokine positive cells as a percentage of all CD8+ T cells (P=0.13 by ANOVA). Thus encouragingly, admixing viral vectors with protein-adjuvant did not affect either T cell quantity or functional “quality”, demonstrating the potential at least in mice for these subunit vaccine platforms to be combined and administered using a single formulation.

Discussion

Immunisation with adenovirus and MVA results in strong CD8 T cell responses and moderate antibody responses, while immunisation with recombinant protein in adjuvant can sometimes result in stronger antibody responses but a relatively poor CD8+ T cell response. We have shown that a vaccination regime comprising three separate, sequential immunisations with adenovirus, then MVA, then protein/Montanide ISA720 (or adenovirus, then protein, then MVA) results in strong combined CD8 T cell responses and antibody responses. This experiment describes mixing protein and Montanide ISA 720 with adenovirus and/or MVA. Using such mixtures an equivalent high level of combined cellular and humoral response, matching that after 3 vaccinations, can be achieved after only two vaccinations.

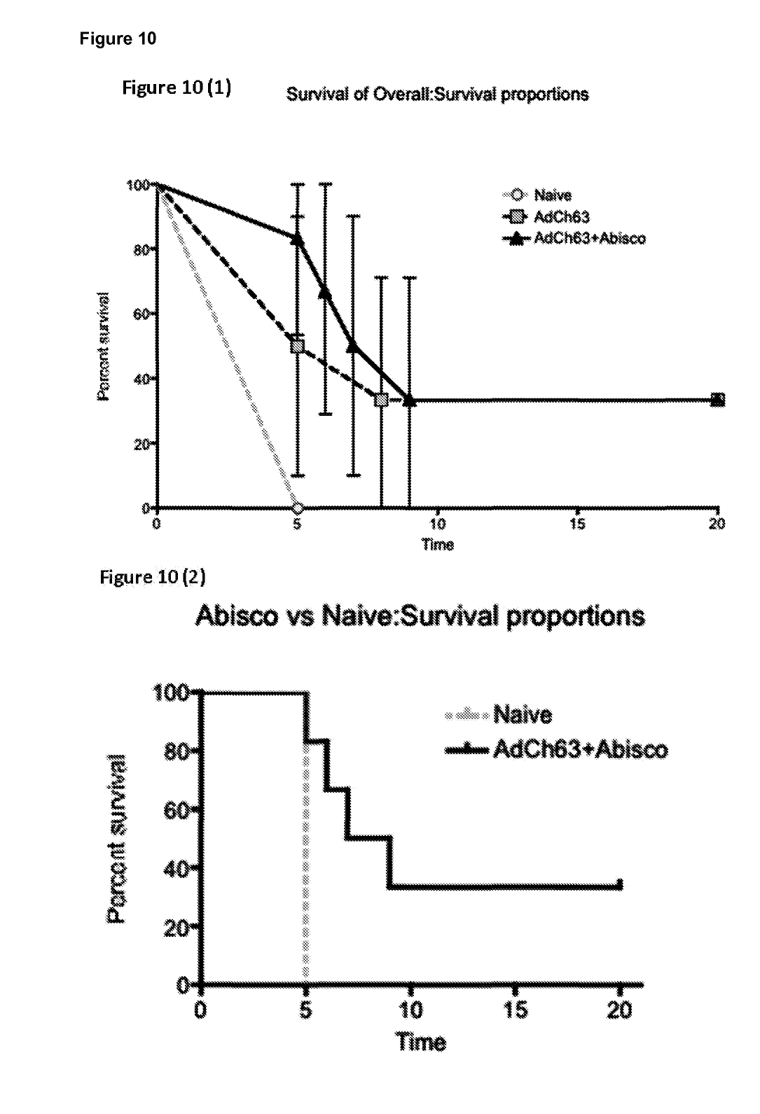

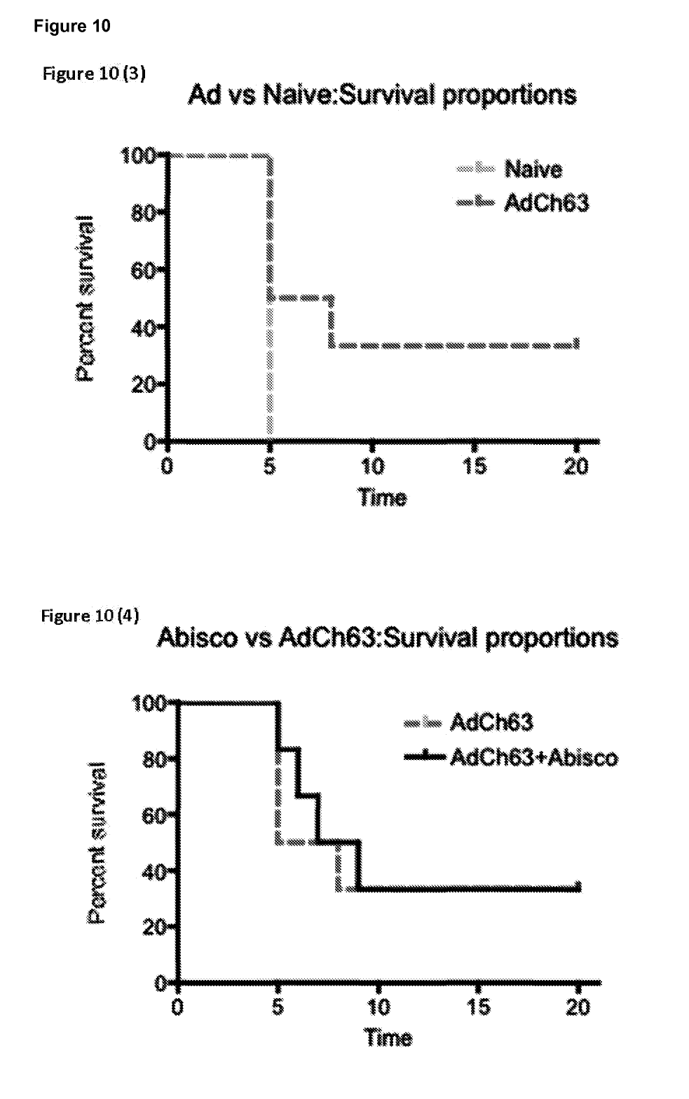

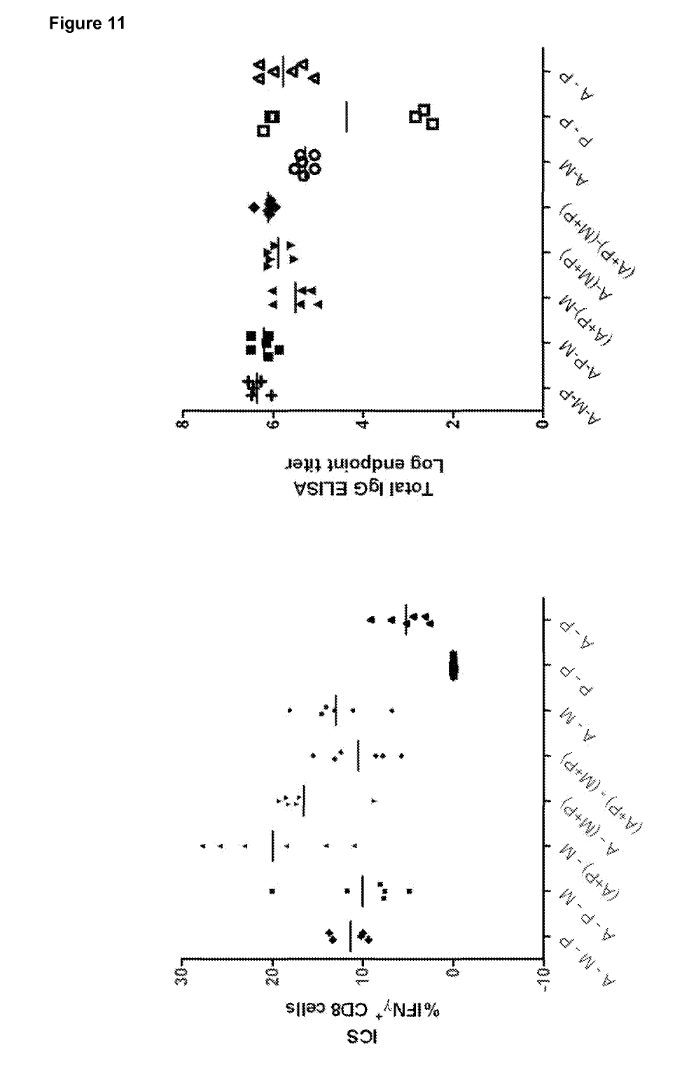

We found that there was no significant difference by Kruskal-Wallis test between the three-immunisation regimes and a two-immunisation regime mixing protein and Montanide ISA 720 with both adenovirus prime and MVA boost (FIG. 10). Interestingly, there was a slight but statistically significant increase in CD8 T cell responses and decrease in antibody responses with the (AP)-M regime relative to A-P-M (P<0.05, ANOVA with Dunn's multiple comparison post-test). Antibody responses trended to be highest with the three component regimes, or when protein-adjuvant was co-administered with both viral vectors. Thus a short two-immunisation regime appears highly immunogenic, and mixing of the viral vectors with protein and adjuvant did not appear to affect potency of the vector-encoded transgene.

### Example 7

### Addition of the Adjuvant ISCOM Matrix Enhances CD8 Responses Induced by MVA Expressing ME.TRAP

We assessed the effect of the adjuvant ISCOM Matrix on the CD8 responses induced by MVA ME.TRAP immunisation. The ME.TRAP transgene consists on the TRAP sequence of P. falciparum, attached to a multi-epitope (ME) string that expresses Pb9, an H2K(d)-restricted epitope (SYIPSAEKI) that is immunodominant in BALB/c mice. To this effect, two groups of 5 BALB/c mice each were immunized bilaterally, intradermally into the ear pinnae with MVA ME.TRAP at a dose of 1×10⁶ pfu/mouse in 25 μl per ear. A control group received the vaccine at the same concentration, resuspended in PBS.

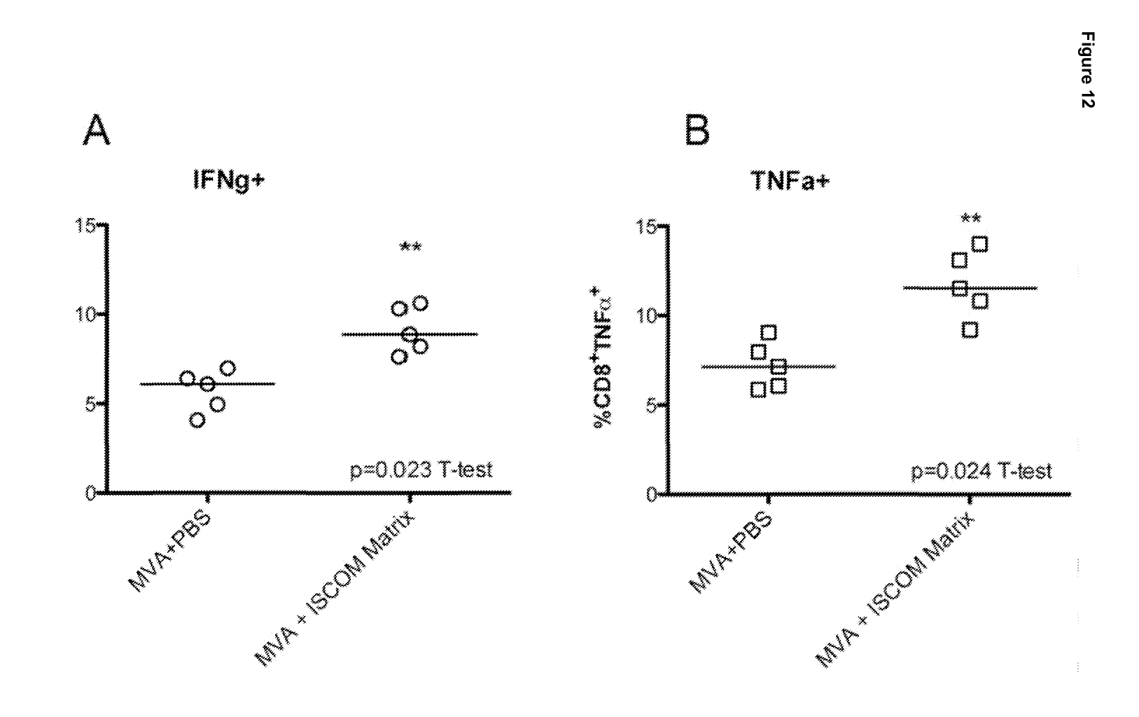

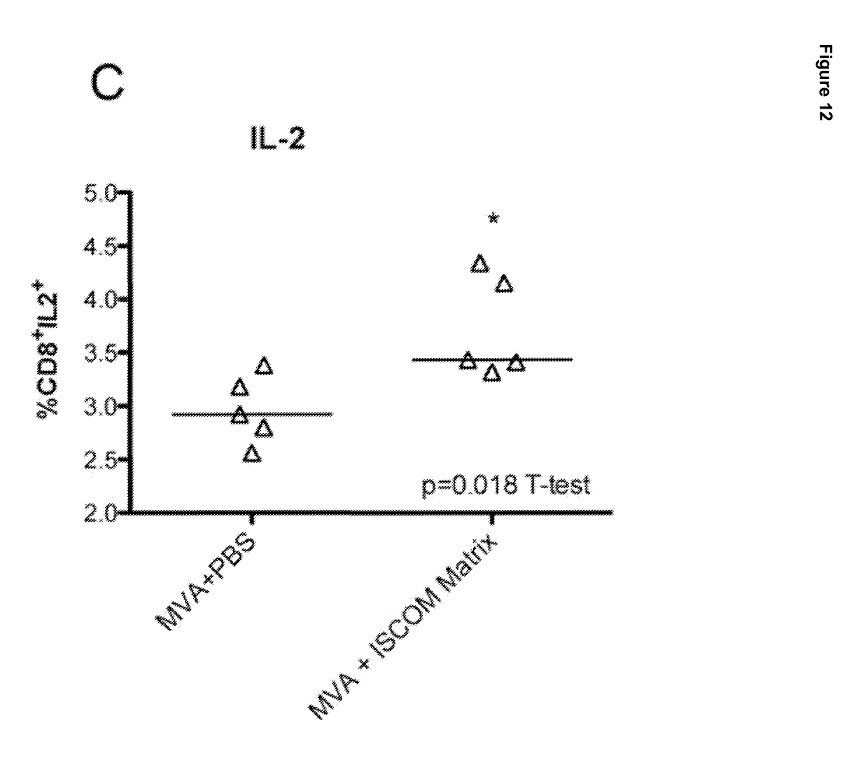

Following three administrations of the adjuvanted and non-adjuvanted MVA ME.TRAP, we noticed an increase in the frequencies of antigen-specific CD8+ T-cells producing IFN-γ and TNF-α and IL-2 in blood. Analysis of multi-functionality revealed an increase in the frequencies of CD8+ cells producing two (IFN-γ, TNF-α) and three cytokines (IFN-γ, IL-2 and TNF-α). (FIG. 12).

### Example 8

### Protein in Various Adjuvants, Including Alum, can Boost an Adenovirus-Primed Response to Achieve High Antibody Titres

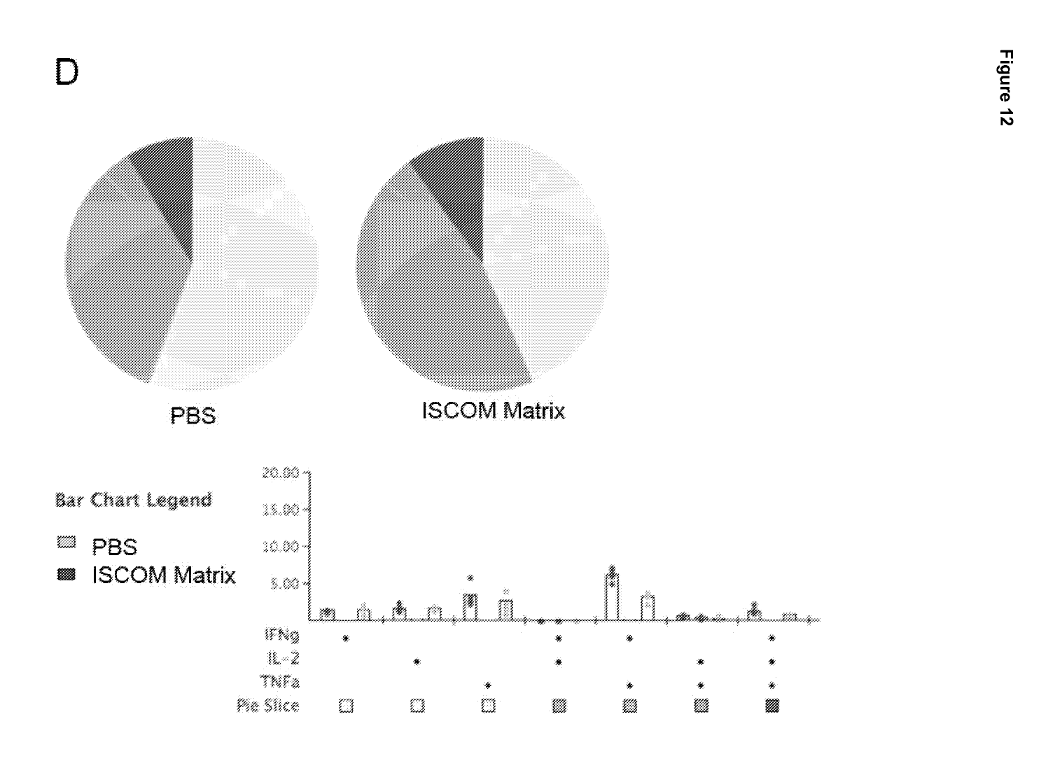

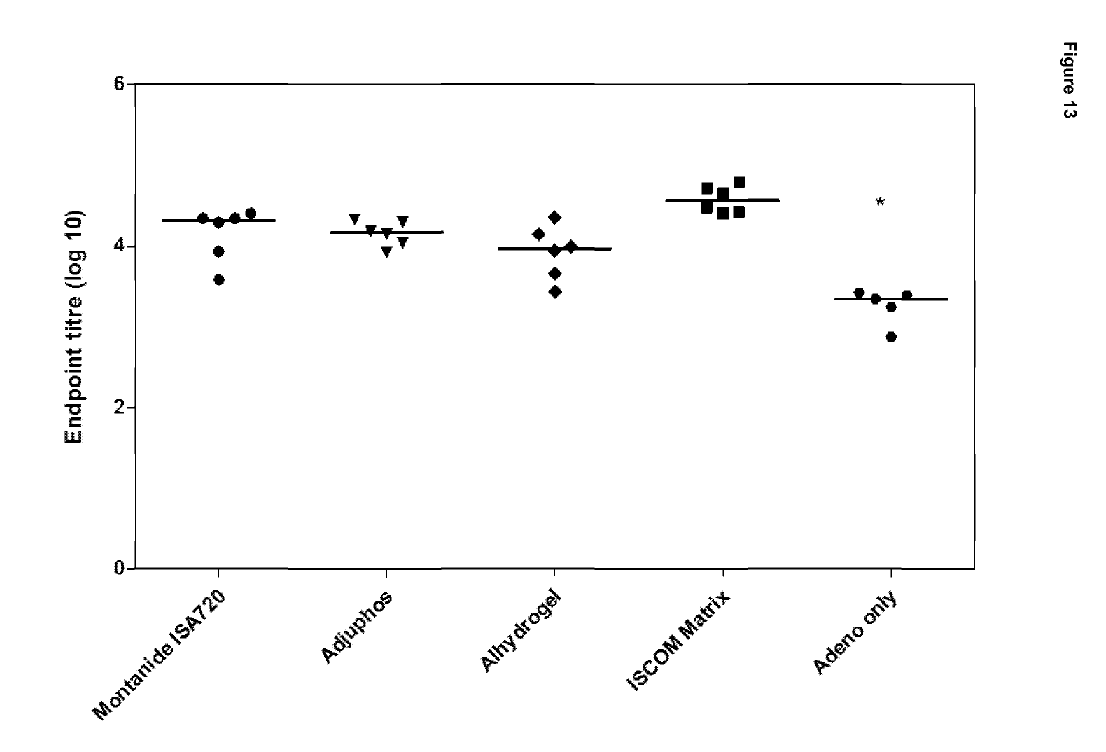

We tested the potency of various adjuvants in boosting the antibody response primed by a single adenovirus injection. Groups of 6 female C57/BL6 mice were immunised with vaccine intramuscularly in a total volume of 50 μl divided equally into each musculus tibialis. Mice were primed at day 0 with 1010 vp of AdHu5 expressing ovalbumin fused to the human tissue plasminogen activator, and boosted on day 56 with 20 μg of ovalbumin protein formulated in adjuvant (1.5 mg/ml of Alhydrogel and Adjuphos per dose, 12 μg of ISCOM Matrix per dose and Monatide ISA720 was given as a 7:3 ratio of adjuvant:antigen). Note that this AdHu5 vector has deletions in both the E1 region and the E3 region ensuring replication incompetence in almost all mammalian cells and increasing the size of the insert that can be used to >5 kb. Total IgG responses to ovalbumin were assayed by ELISA on day 55 (pre-boost) and on day 70, two weeks following the protein in adjuvant boost. All mice had detectable antibody responses on day 55 following the adenoviral prime. After administering the protein in adjuvant vaccine, antibody responses were boosted significantly in all groups compared to the un-boosted control group, as shown in the figure below (* significant versus all adjuvants, p<0.05 ANOVA). There was no significant difference in the fold change of antibody responses expressed as a ratio of pre- to post-boost between the different adjuvant groups (p<0.05 Kruskal-Wallis). Therefore, surprisingly, the alum-based adjuvants were as potent as the ISCOM Matrix and the emulsion (ISA 720) adjuvant for boosting an adenovirus-primed antibody response. (FIG. 13).

### Example 9

### Addition of the Adjuvant ISCOM Matrix at a Higher Dose to Adenovirus Vectored Vaccine Increases the Tcm CD8 Cell Population and Confers Greater Protection Against Malaria Challenge in Mice

We tested the ability of a higher dose of ISCOM Matrix to enhance the protective efficacy of our Ad-ME.TRAP malaria vaccine described in the example 4 above. BALB/c mice (n=6 per group) were immunised bilaterally, intradermally into the ear pinnae, with 5×10⁹ vp Ad-ME.TRAP, with or without ISCOM Matrix adjuvant at a dose of 24 μg/mouse in a vaccination volume of 25 μl per ear.

Two weeks after immunisation, the mice were challenged with malaria by an intravenous delivery of 1,000 sporozoites per mouse. The progress of the infection was monitored by counting parasite numbers on blood smears starting from day 5 post-challenge. Animal survival was recorded and vaccine efficacy measured as the proportion of surviving animals. We found that addition of ISCOM Matrix increased the proportion of surviving mice to 80% compared to 30% observed with the Ad-ME.TRAP vaccine alone (FIG. 14).

We also investigated the effect of the higher ISCOM Matrix dose on the different CD8 T cell populations in peripheral blood and spleen in the same vaccination regime as described above (n=8 animals per group). We assessed the proportion of antigen-specific effector T cells (TE), effector memory T cells (TEM) and central memory T cells TCM, which were distinguished by using CD62L and CD127 surface cell markers. Antigen-specific cells were identified using an MHC tetramer presenting a dominant CD8 T cell Pb9 epitope which is contained within the Ad-ME.TRAP construct.

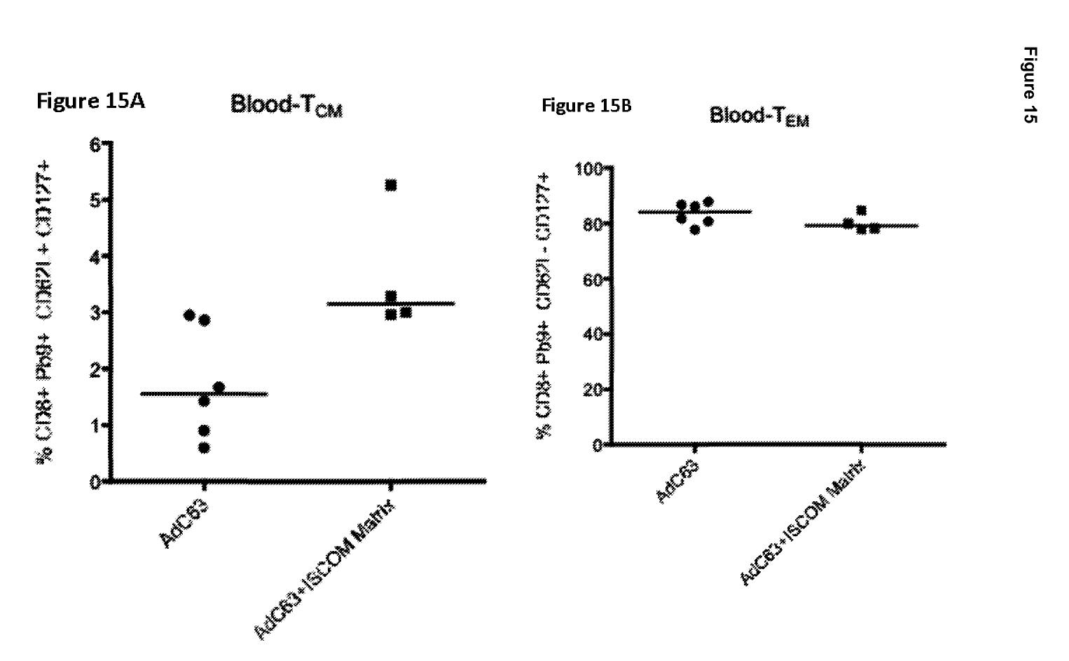

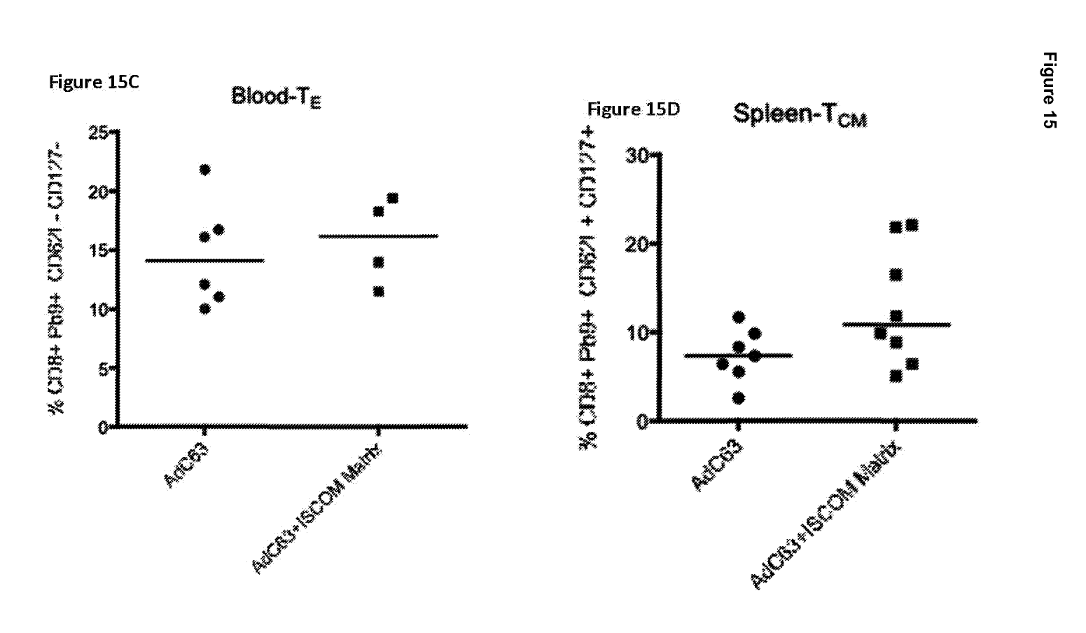

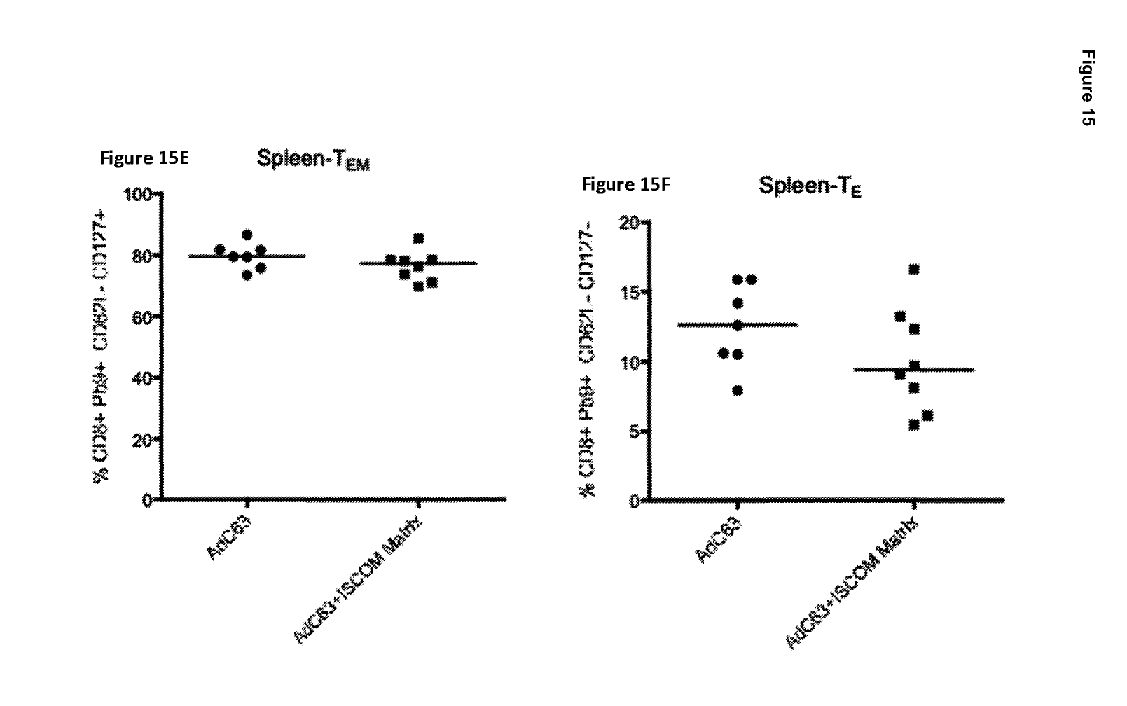

We found that combining ISCOM Matrix with Ad-ME.TRAP did not significantly affect the proportion of TE or TEM cells at either of these two sites. However, the proportion of central memory T cells was found to be significantly higher in the peripheral blood (p<0.09, unpaired t-test) and also showed an increasing trend in the spleen in the group that received the 24 μg dose ISCOM Matrix adjuvant compared to the Ad-ME.TRAP only group (FIG. 15). This finding supports the previously described notion that antigen-specific TCM cell population is associated with enhanced protection by vaccination and increased longevity of vaccine efficacy.

1. A composition comprising:
(a) a modified vaccinia virus ankara (MVA) vector, wherein said MVA vector comprises a nucleic acid sequence encoding an antigen; and
(b) an adjuvant comprising a saponin, or an emulsion.

2. (canceled)

3. The composition of claim 1, wherein the composition further comprises a polypeptide antigen from a pathogenic organism.

4-6. (canceled)

7. The composition of claim 1, wherein the antigen encoded by the nucleic acid sequence is not a Chlamydia sp. antigen, wherein the antigen encoded by the nucleic acid is an antigen selected from the group consisting of: a Plasmodia antigen, an influenza virus antigen, a Mycobacterium tuberculosis antigen, a Mycobacterium bovis antigen, a Mycobacteria antigen, a hepatitis C virus antigen, a flavivirus antigen, a hepatitis B virus antigen, a human immunodeficiency virus antigen, a retrovirus antigen, a Staphylococcus aureus antigen, a Staphylococci antigen, a Streptococcus pneumoniae antigen, a Streptococcus pyogenes antigen, a Streptococci antigen, a Haemophilus influenzae antigen, and a Neisseria meningitides antigen.

8. The composition of claim 1, wherein the MVA vector has an intact A26L gene, wherein the adjuvant comprising a saponin is ISCOM Matrix.

9. (canceled)

10. The composition of claim 1, wherein the emulsion is selected from: Montanide ISA720, Montanide ISA206, Emulsigen, Titermax, and MF59.

11. The composition of claim 1, wherein the adjuvant is a saponin.

12. (canceled)

13. A composition comprising:
(a) an adenovirus vector, wherein said adenovirus vector comprises a nucleic acid sequence encoding an antigen, and wherein the adenovirus is selected from: a group B adenovirus, a group C adenovirus, and a group E adenovirus; and
(b) an adjuvant comprising a saponin, or an emulsion;
wherein the group B adenovirus is not an adenovirus 35, the group C adenovirus is not Ad5 having an intact E3 gene region, and the group E adenovirus is not an adenovirus C7.

14. The composition of claim 13, wherein the group C adenovirus is selected from: adenovirus 6, PanAd3, adenovirus C3, and Ad5 wherein the Ad5 lacks functional E1 and E3 gene regions.

15. The composition of claim 13, wherein the group E adenovirus is selected from: AdCh63, Y25, and AdC68.

16. The composition of claim 13, wherein the adjuvant comprising a saponin is ISCOM Matrix.

17-18. (canceled)

19. The composition of claim 13, wherein the composition further comprises a polypeptide antigen from a pathogenic organism.

20. (canceled)

21. The composition of claim 13, wherein the antigen encoded by the nucleic acid sequence is an antigen from a pathogenic organism.

22. The composition of claim 21, wherein the antigen encoded by the nucleic acid sequence is a malaria antigen.

23. A method of stimulating or inducing an immune response in a subject, comprising administering to the subject a composition according to claim 1.

24. A method of stimulating or inducing an immune response in a subject, comprising administering to the subject a composition according to claim 13.

25-26. (canceled)

27. A method of stimulating or inducing an immune response or preventing or treating an infectious disease in a subject, comprising administering to the subject an MVA vector comprising a nucleic acid sequence encoding an antigen,
wherein the method further comprises administration of a polypeptide antigen or an adenovirus vector comprising a nucleic acid sequence encoding an antigen,
and wherein either one or both of the MVA vector and the polypeptide antigen or adenovirus vector is administered in combination with an adjuvant comprising a saponin, or an emulsion.

28. The method according to claim 27, wherein the MVA vector and the polypeptide antigen are administered to the subject sequentially, in either order.

29-34. (canceled)

35. (canceled)

36. The method according to claim 27, wherein the MVA vector and the adenovirus vector are administered to the subject sequentially, in either order.

37. A method of stimulating or inducing an immune response or preventing or treating an infectious disease in a subject, comprising administering to the subject an adenovirus vector comprising a nucleic acid sequence encoding an antigen, wherein the method further comprises administration of a polypeptide antigen,
and wherein either one or both of the adenovirus vector and the polypeptide is administered in combination with an adjuvant comprising a saponin, an emulsion, or an alum adjuvant.

38. The method according to claim 37, wherein the adenovirus is selected from: adenovirus 6, PanAd3, adenovirus C3, AdCh63, Y25, AdC68, adenovirus C3, and Ad5 wherein the Ad5 has gene deletions in both the E1 and E3 gene regions.

39-41. (canceled)
